(19) United States
(12) Patent Application Publication
(10) Pub. No.: US 2018/0193209 A1
(43) Pub. Date: Jul. 12, 2018

(54) A POLYMER PRODUCT AND PREPARATION THEROF

(71) Applicants: SINGAPORE HEALTH SERVICES PTE LTD., Singapore (SG); NATIONAL UNIVERSITY OF SINGAPORE, Singapore (SG)

(72) Inventors: Lakshminarayanan RAJAMANI, Singapore (SG); Chetna DHAND, Singapore (SG); Seeram RAMAKRISHNA, Singapore (SG); Shouping LIU, Singapore (SG); Roger Wilmer BEUERMAN, Singapore (SG)

(21) Appl. No.: 15/742,289

(22) PCT Filed: Jul. 6, 2016
(86) PCT No.: PCT/US2016/041178

§ 371 Date: Jan. 5, 2018

(30) Foreign Application Priority Data
Jul. 7, 2015 (SG) 10201505350V
Jul. 7, 2015 (SG) 10201505351Q
Jul. 7, 2015 (SG) 10201505355T

(51) Int. Cl. A61F 13/53; D01F 11/02; D01F 11/08; D01F 11/06; D01F 1/10; D01D 5/00; C08L 77/04; C08G 63/08; C08L 71/02; C08L 5/08; C08K 3/10

(52) U.S. Cl. CPC A61F 13/53; D01F 11/02; D01F 11/08; D01F 11/06; D01F 1/103; D01D 5/003; C08K 5/175; C08G 63/08; C08L 71/02; C08L 5/08; C08K 3/10; C08L 2203/12; C08L 77/04

(57) ABSTRACT

The present invention provides a polymer product and a method for preparing the polymer product comprising electrospinning from a dope solution comprising at least one polymer and at least one cross-linking agent to prepare and/or fabricate the polymer product. In certain embodiments, the cross-linking agent comprises at least one catecholamine or at least one polyphenol, wherein the method comprises (i) electrospinning the biocompatible polymer product using a dope solution comprising a polymer and at least one catecholamine or at least one polyphenol and (ii) exposing the polymer product to at least one gaseous alkaline reagent. The dope solutions and polymer products of the invention can further include antimicrobial agents, metal ions, and other substances.

### STATEMENT AS TO RIGHTS TO INVENTIONS MADE UNDER GOVERNMENT-SPONSORED RESEARCH AND DEVELOPMENT

This invention was made with Government support under Grant Nos. NMRC/CBRG/0048/2013 and NMRC/TCR/008-SERI/2013 awarded by the Singapore National Medical Research Council.

### CROSS-REFERENCES TO RELATED APPLICATIONS

The present application claims priority to Singapore Pat. Appl. No. 10201505350V, filed on Jul. 7, 2015; Singapore Pat. Appl. No. 10201505351Q, filed on Jul. 7, 2015; and Singapore Pat. Appl. No. 10201505355T, filed on Jul. 7, 2015; which applications are incorporated herein by reference in their entirety.

### BACKGROUND OF THE INVENTION

The present invention relates to a polymer product and its preparation. Polymeric fibers have various applications including in healthcare and medicine (drug delivery, tissue engineering, regenerative medicine, implants, wound dressings, artificial organ components, biosensors and so on), structural, recreation, textile, optical, electrical, energy and environment (air pollution control and water treatment). In particular, in the area of healthcare and medicine, the large surface area/aspect ratio of polymeric fibers can potentially be utilized to develop valuable products for functionalization or addition of molecules with specific properties.

Electrospinning is a versatile method of fabricating polymeric fibers. The polymer fibers are typically characterized by fiber diameters ranging from several microns down to 100 nm or less. These polymeric fibers may be used to further fabricate products of varying complexity and different three-dimensional shapes.

However, electrospun (ES) fibers fabricated using hydrophilic or water-soluble polymers have a high degree of swelling in aqueous environments, limiting their utility, particularly in healthcare, medicine and also nanodevices. These polymeric fibers and subsequently fabricated products have poor physical and/or mechanical properties, such as strength and stability. Post-spinning cross-linking methods have been used to enhance the properties of such electrospun hydrophilic or water-soluble polymeric fibers. Methods of cross-linking include heating, UV treatment and chemical methods (such as exposure to aqueous or organic solvents). However, all of these methods have limitations. Thermal methods employ high temperature in vacuum but do not necessarily provide adequate improvements in mechanical stability. UV treatment is weak, limited to the surface, and does not penetrate into the polymeric fiber mats. Chemical cross-linking methods also have a number of practical limitations. These methods require multiple steps, are time consuming, have low conjugation efficacy resulting in inadequate stability of the polymers in an aqueous environment and can also alter the properties of the polymers. For instance, existing chemical cross-linking agents may release cytotoxic compounds upon hydrolysis, reduce cellular growth, cause calcification of tissue, and increase the antigenicity of the polymers. Additional steps may be required, for example to remove any cytotoxic residues present. As examples, the most explored cross-linking agents such as formaldehyde, glutaraldehyde, glyceraldehyde, (1-ethyl-3-(3-dimethylaminopropyl)-carbodiimide) (EDC) together with N-hydroxysuccinimide (NHS) and genipin are cytotoxic or also require adverse pH/temperature conditions for cross-linking. All of these drawbacks reduce the applications of the polymers. Accordingly, there remains a need to develop methods for preparing or fabricating polymer products to improve their physical and/or mechanical properties.

### BRIEF SUMMARY OF THE INVENTION

According to a first aspect, the present invention provides a method for preparing a polymer product comprising electrospinning from a dope solution comprising at least one polymer and at least one cross-linking agent to prepare and/or fabricate the polymer product.

According to embodiments wherein the cross-linking agent comprises at least one catecholamine, the method comprises (i) electrospinning from a dope solution comprising at least one polymer and at least one catecholamine to prepare and/or fabricate the polymer product and (ii) exposing the polymer product to at least one gaseous alkaline reagent.

According to a second aspect, the present invention provides a method for preparing a polymer product comprising at least one polyhydroxy antimicrobial agent and/or at least one polyamine antimicrobial agent comprising (i) electrospinning from a dope solution comprising at least one polymer, at least one catecholamine, at least one polyhydroxy antimicrobial agent and/or at least one polyamine antimicrobial agent to prepare and/or fabricate the polymer product and (ii) exposing the polymer product to at least one gaseous alkaline reagent.

According to a third aspect, the present invention provides a method for preparing a polymer product as described above, wherein the dope solution further comprises at least one metal ion, such as a calcium ion.

Any suitable polymer or combination of polymers may be used in the dope solution. The polymer may be a hydrophilic, water-soluble and/or biocompatible polymer. Similarly, any suitable cross-linking agent or combination of cross-linking agents may be used in the dope solution. The cross-linking agent is preferably a biocompatible cross-linking agent. For example, the cross-linking agent may comprise a catecholamine or a polyphenol.

The dope solution comprises at least one polymer and at least one cross-linking agent. It will be appreciated that a suitable solvent is used in the dope solution. The terms dope solution or polymeric dope solution may be used interchangeably.

In some embodiments, the dope solution comprises at least one polymer, at least one catecholamine, at least one polyhydroxy antimicrobial agent and/or at least one polyamine antimicrobial agent.

In some embodiments, the dope solution comprises at least one polymer, at least one catecholamine, and a monovalent cation or a divalent cation. In some embodiments, the cation is a calcium cation.

Any suitable polymer or combination of polymers may be used in the dope solution. The polymer may be a hydrophilic, water-soluble and/or biocompatible polymer. Similarly, any suitable polyhydroxy antimicrobial agent and/or polyamine antimicrobial agent may be used in the dope solution. For example, the polyhydroxy antimicrobial agent may comprise at least one antifungal agent and/or at least one antibacterial agent. Examples of suitable polyamine antimicrobial agent may comprise at least one linear polyamine antimicrobial agent and/or at least one branch polyamine antimicrobial agent.

The present invention also includes a polymer product obtainable by a method according to any one of the preceding claims. In some embodiments, the polymer product comprises at least one polyhydroxy antimicrobial agent and/or at least one polyamine antimicrobial agent. In some embodiments, the polymer product comprises at least one metal ion.

The polymer product may be in the form of a polymeric fiber. The polymeric fiber may subsequently be used to fabricate products of varying complexity and different three-dimensional shapes. The polymer product therefore includes these subsequently products fabricated from polymeric fibers.

### BRIEF DESCRIPTION OF THE DRAWINGS

The invention will be discussed in more detail with reference to the following figures.

FIG. 1 shows a flow chart exemplifying the method for preparing a polymer product. Two dope solutions are represented in the flow chart.

FIG. 2 shows scanning electron microscope (SEM) images of polydopamine-coated gelatin prepared by the Tris-HCl method.

FIG. 3 shows SEM images of different catecholamine-coated gelatin fiber mats and as a control electrospun gelatin fiber mat with no catecholamine (A) with no exposure to and (B) after exposure to gaseous ammonia and carbon dioxide derived from ammonium carbonate. The average diameter ϕ of the polymeric fibers of each respective polymer product is indicated.

FIG. 4 shows SEM images of different catecholamine-coated collagen fiber mats and as a control electrospun collage fiber mats (A) with no exposure to and (B) after exposure to gaseous ammonia and carbon dioxide derived from ammonium carbonate. The average diameter of the polymeric fibers of each respective polymer product is also indicated.

FIG. 5 shows confocal microscopy images of human skin fibroblasts cells cultured on (A) coverslip, coverslip respectively coated with (B) electrospun gelatin (C) electrospun dopamine-coated gelatin [ES Gelatin+DA], (D) electrospun polydopamine-coated gelatin after exposure to gaseous ammonia and carbon dioxide from ammonium carbonate [ES Gelatin (ADM)] and (E) coverslip in the presence of 5 μg/mL nocodazole in the culture medium; and (F) a graph of the viability of human skin fibroblasts cells cultured on various fiber mats from the MTS assay.

FIG. 6 shows (A) the ATR-FTIR of polydopamine-coated gelatin fiber mat prepared by the Tris-HCl method [pDA-Tris-HCl], polydopamine-coated gelatin fiber mat after exposure to gaseous ammonia and carbon dioxide from ammonium carbonate [pDA-ADM] and electrospun gelatin [ES gelatin], (B) image of the water drop on electrospun gelatin fiber mat after 20 seconds (C) image of the “levelled-off” water drop on electrospun polydopamine-coated gelatin fiber mat after exposure to gaseous ammonia and carbon dioxide from ammonium carbonate; and (D) RP-HPLC chromatograms of (i) dopamine, polydopamine coating prepared by: (ii) ammonium carbonate diffusion method according to one embodiment of the present invention, and (iii) Tris-HCl method.

FIG. 7 shows confocal microscope images of (A) electrospun gelatin fiber mats, (B) electrospun dopamine-coated gelatin fiber mat after exposure to gaseous ammonia and carbon dioxide from ammonium carbonate (the bar represents 10 μm); and (C) the λ-scans of electrospun gelatin fiber mats and electrospun dopamine-coated gelatin fiber mats from dope solutions of different dopamine concentrations after exposure to ammonium carbonate.

FIG. 8 shows a flow chart exemplifying the method for preparing a polymer product comprising a polyhydroxy antimicrobial agent. Two dope solutions are represented in the flow chart.

FIG. 9 shows scanning electron microscope images exhibiting the morphology of the electrospun polydopamine cross-linked gelatin antimicrobial fiber mats after ammonium carbonate exposure. The antimicrobial agents used are (A) amphotericin B, (B) caspofungin and (C) vancomycin.

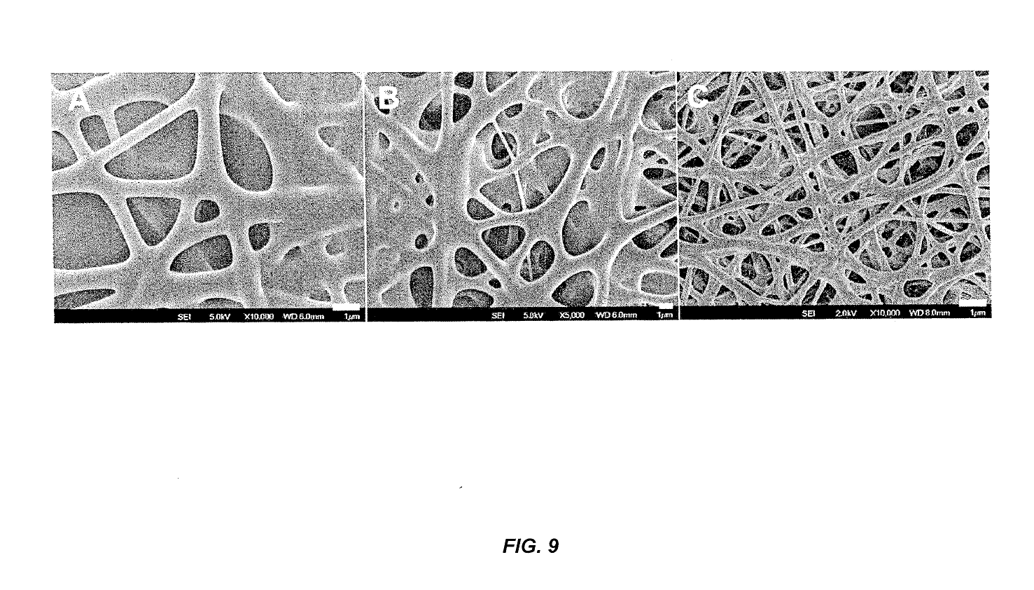

FIG. 10 shows (A) RP-HPLC profile (i) of 50 μg/mL of pure amphotericin B (AmB 50 μg/mL) and (ii) showing the amount of amphotericin B released from fiber mat electrospun from dope solution comprising 10% w/v gelatin, dopamine (2% w/w of gelatin (polymer) in the dope solution) and amphotericin B (0.5% w/w of gelatin (polymer) in the dope solution and crosslinked by exposure to ammonium carbonate following sonication (Gelatin_DA_AmB); (B) Release profile of daptomycin from fiber mat electrospun from dope solution comprising 10% w/v gelatin, dopamine (2% w/w of gelatin (polymer) in the dope solution) and daptomycin (0.5% w/w of gelatin (polymer) in the dope solution) after crosslinking by exposure to ammonium carbonate; (C) RP-HPLC profile of 25 μg/ml caspofungin (CF); and (D) RP-HPLC profile showing the amount of caspofungin released from fiber mat electrospun from dope solution comprising 10% w/v gelatin, dopamine (2% w/w of gelatin (polymer) in the dope solution) and amphotericin B (0.5% w/w of gelatin (polymer) in the dope solution and crosslinked by exposure to ammonium carbonate following sonication (gelatin_pDA_CF).

FIG. 11 shows long-term efficacy of electrospun polydopamine cross-linked gelatin fiber mats with: (A) antifungal agents (amphotericin B or caspofungin); (B) antibacterial agents (daptomycin or vancomycin) after exposure to ammonium carbonate; and (C) long-term efficacy of electrospun polydopamine cross-linked gelatin fiber mats with amphotericin B without (As-spun) and after (XL) exposure to ammonium carbonate.

FIG. 12 shows scanning electron microscopy images exhibiting the morphology of electrospun fiber mats immersed in PBS: (A) Electrospun pristine gelatin fiber mats immersed in PBS for 5 h; Electrospun polydopamine cross-linked gelatin fiber mats after exposure to ammonium carbonate immersed in PBS for (B) 1 week, and (C) 2 weeks.

FIG. 13 shows scanning electron microscopy images exhibiting the morphology of electrospun polydopamine cross-linked gelatin fiber mats with: (A)-(E) amphotericin B; or (F)-(J) caspofungin after exposure to ammonium carbonate immersed in PBS for 1, 2, 3, 4 and 5 weeks.

FIG. 14 shows the morphological analysis of electrospun mats prepared under various conditions. (a) pristine collagen mats. (b) and (c) collagen mats containing 10% DA and NE, respectively. (d) and (e) mats shown in (b) and (c) after exposure to (NH₄)₂CO₃. Scale bar=1 μm. Photographs of the mats are shown in the bottom panel. Note the increased amount of welded junctions and coloration after ADM in both the catecholamines loaded mats. Effect of CaCl₂ on the morphology of electrospun collagen containing (f) DA and (g) NE. Note the complete coating of polycatecholamines along the entire surface of the mats as well as significant color changes in the mats. (h) and (i) Morphology of electrospun mats in (f) and (g) after exposure to (NH₄)₂CO₃. Note the intense brown coloration of the mats containing DA. Insets in (h) and (i) are high resolution TEM images displaying the formation of CaCO₃ particles after ADM.

FIG. 15 shows selected area electron diffraction patterns for the identification mineral phase present in (a) Coll_pDA_Ca and (b) Coll_pNE_Ca mats. The inset shows the TEM images of the mats containing electron dense CaCO₃ particles embedded inside the collagen matrix.

FIG. 16 shows the surface wettability (θ_{static}) of electrospun collagen nanofibers prepared under various conditions determined by dynamic contact angle measurements.

FIG. 17 shows the surface wettability of electrospun collagen mats prepared under various conditions. (a) and (c) show the time-dependent changes in the advancing contact angle for mats containing DA and NE, respectively. The symbols indicate average data points at every 10 seconds and solid or broken lines represent the model fit. (b) and (d) compares the static water contact angle (θ_{static}) determined from the dynamic contact angle measurements. *p≤0.05, **p<0.01, ***p<0.001 and ****p<0.0001 compared to pristine collagen scaffold by t-test or 1-way ANOVA.

FIG. 18 shows the XPS characterization of electrospun mats prepared under various conditions. High resolution C is spectra of (a) ES_Coll, (b) Coll_DA_Ca, (c) Coll_NE_Ca, (d) Coll_pDA_Ca and (e) Coll_pNE_Ca. High resolution N is spectra of (f) ES_Coll, (g) Coll_DA_Ca, (h) Coll_NE_Ca, (i) Coll_pDA_Ca and (j) Coll_pNE_Ca. (k)-(m) show the high resolution Ca 2p, Cl 2p and O 1s spectra for the electrospun mats.

FIG. 19 shows the mechanical properties of electrospun collagen mats. Stress-strain curves of the electrospun mats prepared under various conditions containing (a) DA and (b) NE. Peak stress (σ) and tensile stiffness (E′) values determined from the curves are shown in bar graphs for the mats containing (c) DA and (d) NE. E′ is shown in log₁₀ units for a better comparison. The increase in elongation at break (ε_{b}) and toughness (J_{lc}) are shown for mats containing (e) DA and (f) NE. Note the marked increase in elasticity and stiffness of mineralized collagen mats cross-linked with polycatecholamines.

FIG. 20 shows the fracture morphology of collagen mats. SEM images of electrospun mats after tensile testing. (a) ES_Coll, (b) Coll_DA, (c) Coll_NE, (d) Coll_pDA, (e) Coll_pNE, (f) Coll_DA_Ca, (g) Coll_NE_Ca, (h) Coll_pDA_Ca and (i) Coll_pNE_Ca. Scale bar=1 μm. Note that the mineralized collagen mats (h) and (i) displayed extensive roughness and corrugation indicating considerable increase in tensile strength, stiffness and toughness.

FIG. 21 shows the photoluminescence of collagen mats. Confocal fluorescence images showing the photoluminescent properties of collagen mats. (a) ES_Coll, (b) Coll_DA_Ca, (c) Coll_NE_Ca, (d) Coll_pDA_Ca and (e) Coll_pNE_Ca. The excitation wavelengths used are 405 nm (blue) and 488 nm (green). Scale bar=10 μm. (f) Average blue and green fluorescence intensities obtained from various electrospun collagen.

FIG. 22 shows the cytocompatibility of collagen mats. (a) hFob cell viability (quantified from live/dead cell intensity) cultured on various scaffolds and TCP. Confocal fluorescence images of hFob cells cultured on various collagen mats stained with calcein FDA after 3, 6 and 9 days p.s. (b) ES_Coll, (c) Coll_DA_Ca, (d) Coll_NE_Ca, (e) Coll_pDA_Ca and (f) Coll_pNE_Ca. Top (xy-scan) and side views (z-stack) are represented by ‘t’ and ‘s’, respectively. Scale bar=20 μm.

FIG. 23 shows confocal fluorescence images of hFob cells cultured on various collagen mats with F-actin stained with far-red dye 3, 6 and 9 days p.s. (a) ES_Coll, (b) Coll_DA_Ca, (c) Coll_NE_Ca, (c) Coll_pDA_Ca and (e) Coll_pNE_Ca. Top (xy-scan) and side views (z-stack) are represented by ‘t’ and ‘s’, respectively. Scale bar=20 μm. The black scale bar=44.2 μm.

FIG. 24 shows hFOB cell proliferation monitored by MTT assay (A) and cell differentiation assay monitored by ALP activity (B).

FIG. 25 shows hFob cell proliferation and ALP activity on various composite collagen mats. (a) Metabolic activity of hFob cells assessed by MTS assay at various time points. Data are reported as mean±standard deviation (n=5). (b) Intracellular ALP activity of hFob cells assessed by pNPP assay at various points. ALP activity was normalized by the cell number (μmol p-Nitrophenol of hFob cells/h/cell number) and reported. Data are reported as mean±standard deviation (n=3). Note the marked increase in osteoblasts proliferation and differentiation on mineralized mats containing polycatecholamines.

FIG. 26 shows osteoblast proliferation and differentiation on electrospun polymer products. FIG. 26(a) shows hFob proliferation after seeding on various electrospun collagen mats at various time intervals. FIG. 26(b) shows hFob differentiation after seeding on various electrospun collagen mats at various time intervals.

FIG. 27 shows Calcium deposits on osteoblasts cultured on various ES collagen mats at various time intervals. Note the extensive staining on ES collagen doped with catecholamines and calcium after gaseous ammonia treatment (indicated by white circle).

FIG. 28 shows ARS staining of calcium deposition on collagen mats. Determination of calcium deposition on various collagen mats by ARS staining. (a) TCP, (b) ES_Coll, (c) Coll_DA_Ca, (d) Coll_NE_Ca, (e) Coll_pDA_Ca and (f) Coll_pNE_Ca. Scale bar=50 μm. (g) Quantitative estimation of calcium deposition by cetyl pyridinium bromide assay at 14 days p.s. Data are reported as mean±standard deviation from three independent experiments.

FIG. 29 shows morphology of osteoblasts cultured on various ES collagen after the third and ninth days of culture time.

FIG. 30 shows the morphology of hFob seeded on various collagen mats. Representative SEM images showing the features of hFOb after 9 days p.s. (a) TCP, (b) ES_Coll, (c) Coll_DA_Ca, (d) Coll_NE_Ca, (e) Coll_pDA_Ca and (f) Coll_pNE_Ca. Scale bar=10 μm.

FIG. 31 shows the expression of key osteogenic proteins by hFob seeded on various collagen mats. Representative immune staining images showing the expression of osteocalcin (OCN), osteopontin (OPN) and bone matrix protein (BMP) after 11 days p.s. (a) TCP, (b) ES_Coll, (c) Coll_DA_Ca, (d) Coll_NE_Ca, (e) Coll_pDA_Ca and (f) Coll_pNE_Ca. Scale bar=20 μm. (g) quantitative expression of OPN by Western blotting.

FIG. 32 shows the quantification of the mean fluorescence (violet) intensity of osteogenic markers in hFob seeded on various scaffolds. (a) OPN and (b) OCN and (c) BMP-2. Each bar represents mean±SD (n=3).

FIG. 33 shows the effect of Ca²⁺ on dopamine polymerization at alkaline pH. UV spectra of (A) dopamine Tris-HCl pH 8.5 and (B) dopamine in Tris-HCl containing 25 mM CaCl₂.

FIG. 34 shows the biocompatibility and microbiological evaluation of Gel_pDA mats. Confocal images showing the morphology of primary human dermal fibroblasts (hDFs) seeded on (a) pristine ES_Gel, (b) Gel_DA, and (c) Gel_pDA. Scale bar=20 μm. (d) MTS assay confirming the metabolic activity of hDFs seeded on various ES mats and coverslips (CS). Photographs of disc diffusion assays showing the efficacy of Gel_pDA mats loaded with vancomycin (e) and caspofungin (f). Scale bar=1 cm. Durability of vancomycin (g) and caspofungin (h) antibiotic-loaded Gel_pDA mats to leaching. Note that the antibiotic-loaded Gel_pDA mats displayed excellent durability when compared with the silver-based wound dressing Aquacel®Ag.

FIG. 35 shows the morphology of antibiotics loaded ES gelatin mats containing DA before and after ADM exposure. (a) and (c) contain vancomycin whereas (b) and (d) contain caspofungin as the antibiotics. The amount of antibiotics loaded was 0.5% (w/w of the polymer). Note that the fiber diameters in vancomycin-loaded mats is significantly decreased due to differences in solvent conditions.

FIG. 36 shows in vivo wound healing efficacy of Vanco_Gel_pDA mats in a porcine burn injury model. Digital photographs showing the wound closure during the course of the treatments. (a) Untreated, (b) ES_Gel, (c) Vanco_Gel_pDA mats, and (d) Aquacel® Ag wound dressings. For clarity, only photographs of the wounds before and after treatment are shown. (e) Temporal changes in the wound closure area after various treatments during the entire course of the study. (f) Quantitative estimation of the wound closure area for treated and untreated wounds. Note the increased wound closure for Vanco_Gel_pDA mats when compared with pristine and silver-based dressings.

FIG. 37 shows the in vivo assessment of the wound healing properties of pristine ES_Gel and Vanco_Gel_pDA mats in a deep dermal porcine burn wound model.

### DETAILED DESCRIPTION OF THE INVENTION

### I. General

The present invention provides new methods for electrospinning of biocompatible polymers. For example, the methods can be used for the generation of durable antimicrobial or mineralized scaffolds for advanced wound dressings and bone tissue engineering. The methods utilize a polycatecholamine coating and relatively mild conditions for the preparation of electrospun nanofiber scaffolds. The methods employ no organic solvents, toxic additives, or extreme temperatures, and are therefore readily applicable to manufacturing practices. Furthermore, the methods confer additional benefits to the procured nanofibrous scaffolds, including excellent surface smoothness, desirable mechanical properties, the ability to anchor peptide antibiotics to the nanofiber surface for an extended period of time, inherent nanofiber photoluminescent characteristics, and biocompatibility both in vitro and in vivo. The polycatecholamine coating can also be mineralized to generate osteoconductive scaffolds.

### II. Definitions

As used herein, the term “electrospinning” refers to a process in which a high voltage is used to create an electrically charged jet of polymer fluid, such as a polymer solution, which dries or solidifies to generate polymer fibers. Systems for electrospinning generally include a syringe, a nozzle, a pump, a high-voltage power supply, and a grounded collector. A high voltage power supply is connected to the orifice of the needle at one end and to the grounded collector on the other end.

As used herein, a “biocompatible” substance (for example polymer or cross-linking agent) is one that does not generally cause significant adverse reactions (e.g. toxic or antigenic responses) to cells, tissues, organs or the organism as a whole, of example, whether it is in contact with the cells, tissues, organs or the organism as a whole, for example, whether it is in contact with the cells, tissues, organs or localized within the organism, whether it degrades within the organism, remains for extended periods of time, or is excreted whole. A biocompatible substance (e.g., a biocompatible polymer) may be selectively compatible in that it exhibits biocompatibility with certain cells, tissues, organs or even certain organisms. For example, the biocompatible substance may be selectively biocompatible with vertebrate cells, tissues and organs but toxic to cells from pathogens or pathogenic organisms. In some circumstances, the biocompatible substance may also be toxic to cells derived from tumors and/or cancers.

As used herein, the terms “comprising” or “including” are to be interpreted as specifying the presence of the stated features, integers, steps or components as referred to, but do not preclude the presence or addition of one or more features, integers, steps or components, or groups thereof. However, in context with the present disclosure, the terms “comprising” or “including” also includes “consisting of” The variations of the word “comprising,” such as “comprise” and “comprises,” and “including,” such as “include” and “includes,” have correspondingly varied meanings.

As used herein, the terms “dope solution” and “polymeric dope solution” are used interchangeably to refer to a material used in the electrospinning process. Electrospinning a substance “from a dope solution” means that the substance is present in the dope solution before the electrospinning process is initiated.

As used herein, “microfiber” refers to a fiber with a diameter no more than 1000 micrometers.

As used herein, “nanofiber” refers to a fiber with a diameter no more than 1000 nanometers.

As used herein, “polyhydroxy antimicrobial agent” refers to an antimicrobial agent with two or more hydroxyl groups within its chemical structure. The two or more hydroxyl groups may preferably be non-ionizable hydroxyl groups.

### III. Electrospinning Methods for Preparation of Composite Polymer Products

According to a first aspect, the present invention provides a method for preparing a polymer product comprising electrospinning from a dope solution comprising at least one polymer and at least one cross-linking agent to prepare and/or fabricate the polymer product.

The dope solution comprises at least one polymer and at least one cross-linking agent in a suitable solvent. It will be appreciated that the solvent should not be aqueous in certain instances, particularly for a hydrophilic and/or water-soluble polymer product. In such instances, the solvent may be an organic solvent. Examples of suitable solvents include but are not limited to 2,2,2-trifluoroethanol (TFE) and hexafluoroisopropanol (HFIP).

In some embodiments, the dope solution comprises a polymer, a cross-linking agent such as a catecholamine, an organic solvent such as TFE and HFIP, and water. Water can be included to promote the solubility of metal salts, hydrophilic antibiotics, and other substances as described in more detail below. In some such embodiments, the dope solution is formed using a mixture containing the solvent in an amount of at least around 80% (v/v) and water in an amount of at most around 20% (v/v). The mixture can contain, e.g., around 5, 10, 15, or 20% (v/v) water and an organic solvent such as TFE or HFIP. As a non-limiting example, the dope solution can be formed using a mixture of water:HFIP (5%:95%) or water:HFIP (10%:90%).

Any suitable polymer or combination of polymers may be used in the dope solution. The polymer may be a hydrophilic, water-soluble and/or biocompatible polymer. Examples of polymers include, but are not limited to, gelatin, collagen, bovine serum albumin, casein, zein, laminin, polyvinyl alcohol (PVA), polyacrylic acid and chitosan. Other synthetic biocompatible polymers, such as poly lactide (PLA), poly(ε-caprolactone) (PCL), polyethylene oxide (PEO), poly-(lactide-co-glycolide) (PLGA), can also be used. The dope solution may comprise a combination of polymers. In particular, the dope solution may comprise gelatin and/or collagen.

Similarly, any suitable cross-linking agent or combination of cross-linking agents may be used in the dope solution. The cross-linking agent is preferably a biocompatible cross-linking agent. For example, the cross-linking agent may comprise a catecholamine, a polyphenol, or a combination thereof.

It will be appreciated that the cross-linking agent becomes distributed throughout the polymer product and cross-links polymeric fibers. It will be further appreciated that this improves the physical and/or mechanical properties of the polymer product.

It will be further appreciated that the method of the present invention does not require any harsh and/or adverse pH, heat or pressure conditions.

Any suitable polyphenol may be used as the cross-linking agent in the method according to the first aspect of the invention and any suitable catecholamine may be used as the cross-linking agent in the method according to any aspect of the invention. For example, suitable polyphenols include but are not limited to hydroquinone, phloroglucinol, pyrogallol, gallic acid, nordihydroguaiaretic acid, γ-mangostin, and α-mangostin. In some embodiments, the polyphenol is selected from hydroquinone, phloroglucinol and α-mangostin.

In addition, examples of suitable catecholamines include but are not limited to adrenalone, carbidopa, colterol, L- or D-dihydroxy phenylalanine (dopa), dimethyldopa, dioxifedrine, dioxethedrin, dopamine, 5-hydroxydopamine hydrochloride, dobutamine, dopamantine, dopexamine, droxydopam, norepinephrine, α-methylnorepinephrine, ethylnorepinephrine, etilveodopa, isoetharine, hexaprenaline, N-methyladrenalone, norbudrine, nordefrin, oxidopamine and enterobactin.

In some embodiments, the catecholamine is selected from adrenalone, carbidopa, colterol, dihydroxy phenylalanine (dopa), dimethyldopa, dioxifedrine, dioxethedrin, dopamine, dobutamine, dopamantine, dopexamine, droxydopam, norepinephrine, α-methylnorepinephrine, ethylnorepinephrine, etilveodopa, isoetharine, hexaprenaline, N-methyladrenalone, norbudrine, nordefrin, oxidopamine, and enterobactin.

According to a particular aspect wherein the cross-linking agent comprises at least one catecholamine, the method comprises (i) electrospinning from a dope solution comprising at least one polymer and at least one catecholamine to prepare and/or fabricate the polymer product and (ii) exposing the polymer product to at least one gaseous alkaline reagent.

The polymer product may be treated to substantially remove any residual solvent from the dope solution prior to exposing the polymer product to the gaseous alkaline solution. For example, the treatment can include drying the polymer product at ambient pressure or at reduced pressures in a vacuum.

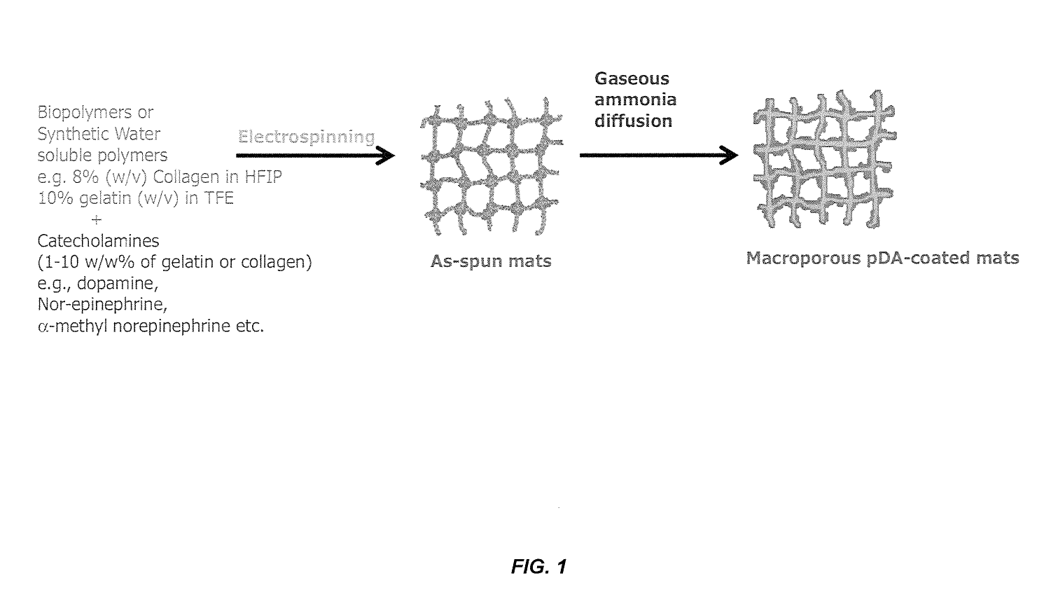

It will be appreciated that the catecholamine becomes distributed throughout the polymer product after electrospinning. Catecholamines form cross-links throughout the polymer product under upon exposure to the gaseous alkaline solution. A flow chart exemplifying the method is illustrated in FIG. 1. It will be appreciated that exposure to a gaseous alkaline reagent results in a higher degree of cross-linking in the polymer product compared to no exposure to the gaseous alkaline reagent.

It will be further appreciated that because the cross-linking process is performed without exposing the polymer product to an aqueous environment, the process may be applied to any nanofibers derived from hydrophilic and/or water-soluble polymers.

Exposing the polymer product to at least one gaseous alkaline reagent may occur in the presence of a buffering agent. The buffering agent serves to prevent a rapid increase in the pH. As a non-limiting example, the buffering agent can be a soluble ammonium salt (e.g., ammonium carbonate, ammonium chloride, ammonium sulfate, and the like).

Any suitable gaseous alkaline reagent may be used. For example, the gaseous alkaline reagent may comprise but is not limited to gaseous ammonia. The gaseous ammonia may be derived from any suitable source. For example, the gaseous ammonia may be derived from ammonium carbonate solid, ammonium hydroxide and/or liquid ammonia. In particular, the ammonium carbonate solid comprises ammonium carbonate powder. For convenience, the method of producing a polymer product by electrospinning a dope solution comprising a polymer and catecholamine and subsequent exposure to gaseous ammonia is referred to as ammonia diffusion method (ADM).

Solid ammonium carbonate disintegrates to form gaseous ammonia (NH₃) and carbon dioxide (CO₂). Even after the polymer product has been dried, some liquid would still remain within the polymer product. The NH₃ and CO₂ dissolve in the liquid. The dissolved NH₃ increases the pH of the liquid whereas the dissolution of CO₂ produces carbonic acid which is deprotonated in the presence of ammonia to from bicarbonate/carbonate ions. The formation of bicarbonate/carbonate ions also prevents an abrupt rise in the pH due to the presence of ammonia.

The ammonia diffusion method can be conducted by placing the polymer product in a sealed container (e.g., a desiccator or other vessel) together with solid ammonium carbonate for a period of time sufficient for catecholamine polymerization. Typically, a polymer product will be stored with the ammonium carbonate for a period of time ranging from a few minutes to several hours, or longer, at temperatures ranging from around 20° C. to around 40° C. For example, the polymer product can be stored with the solid ammonium carbonate for 6 hours, or 12 hours, or 24 hours. The storage step can be conducted, for example, at 20° C. or 25° C.

According to a second aspect, the present invention provides a method for preparing a polymer product comprising at least one polyhydroxy antimicrobial agent and/or at least one polyamine antimicrobial agent comprising (i) electrospinning from a dope solution comprising at least one polymer, at least one catecholamine, at least one polyhydroxy antimicrobial agent and/or at least one polyamine antimicrobial agent to prepare and/or fabricate the polymer product and (ii) exposing the polymer product to at least one gaseous alkaline reagent.

The dope solution comprises at least one polymer, at least one catecholamine, at least one polyhydroxy antimicrobial agent and/or at least one polyamine antimicrobial agent in a suitable solvent (which solvent is described above). Any suitable polymer or combination of polymers, as described above, may be used in the dope solution. Exposure of the electrospun polymer product to gaseous alkaline reagents can be conducted as described above.

The catecholamine and the polyhydroxy/polyamine antimicrobial agent(s) become distributed throughout the polymer product and cross-links polymeric fibers. It will be appreciated that this improves the physical and/or mechanical properties of the polymer product. It will be appreciated that a polymer product with polyhydroxy antimicrobial agent and/or polyamine antimicrobial agent comprises a higher degree of cross-linking in the polymer product compared to a polymer product without polyhydroxy antimicrobial agent and/or polyamine antimicrobial agent. The polyhydroxy antimicrobial agent and/or polyamine antimicrobial agent form cross-links throughout the polymer product upon exposure to the gaseous alkaline solution and the polymer product is thus capable of sustained delivery of the polyhydroxy antimicrobial agent and/or polyamine antimicrobial agent over an appropriate or desired period of time.

Any suitable catecholamine and any suitable polymer, e.g., those described above, can be used. Any suitable polyhydroxy antimicrobial agent or a combination of polyhydroxy antimicrobial agents may be used according to any aspect of the invention. The polyhydroxy antimicrobial agent may comprise at least one antifungal agent and/or at least one antibacterial agent. For example, suitable antifungal agent includes but is not limited to natamycin, nystatin, amphotericin B or caspofungin. In addition, examples of suitable antibacterial agent include but are not limited to vancomycin, polymycin B, daptomycin, ramoplanin A2, ristomycin monosulfate, bleomycin sulfate, phleomycin, amikacin, streptomycin, gentamycin, kanamycin, tobramycin, azithromycin, dirtythromycin, rifampicin, rifamycin, rifapentine, rifaximin, clarithromycin, clindamycin, kendomycin, bafilomycin, chlortetracycline, doxorubicin, doxycycline, tetracycline, 1-deoxynojirimycin, 1-deoxymannojirimycin, and N-methyl-1-deoxynojirimycin.

Any suitable polyamine antimicrobial agent or a combination of polyamine antimicrobial agent may be used according to any aspect of the invention. The polyamine antimicrobial agent may be either linear or branched. For example, suitable polyamine antimicrobial agents include but are not limited to ε-polylysine, poly-L-lysine, poly-D-lysine, poly-L-ornithine, and linear and branched polyethyleneimines. In some embodiments, the polyamine antimicrobial agent is selected from ε-polylysine, poly-L-lysine, poly-D-lysine, and poly-L-ornithine.

According to a third aspect, the present invention provides a method for preparing a polymer product comprising at least one polyhydroxy antimicrobial agent and/or at least one polyamine antimicrobial agent comprising (i) electrospinning from a dope solution comprising at least one polymer, at least one catecholamine, at least one polyhydroxy antimicrobial agent and/or at least one polyamine antimicrobial agent to prepare and/or fabricate the polymer product and (ii) exposing the polymer product to at least one gaseous alkaline reagent.

The dope solution comprises at least one polymer, at least one catecholamine, and at least one metal ion in a suitable solvent (which solvent is described above). Any suitable polymer or combination of polymers, as described above, may be used in the dope solution. Exposure of the electrospun polymer product to gaseous alkaline reagents can be conducted as described above. In some embodiments, the dope solution further comprises at least one polyhydroxy antimicrobial agent and/or at least one polyamine antimicrobial agent.

Any suitable catecholamine and any suitable polymer, e.g., those described above, can be used. Any soluble metal salt can be used in the methods of the invention. In some embodiments, the metal ion is a cation (e.g., a monovalent cation, a divalent cation, or a trivalent cation). In some embodiments, the metal ion is a transition metal ion. Transition metals ions include, for example, titanium ions (e.g., Ti^{3+/}Ti⁴⁺), manganese ions (e.g., Mn²⁺), iron ions (e.g., Fe²⁺ or Fe³⁺), cobalt ions (e.g., Co²⁺), nickel ions (e.g., Ni²⁺), copper ions (e.g., Cu²⁺), zinc ions (e.g., Zn²⁺), ruthenium ions (e.g., Ru³⁺), rhodium ions (e.g., Rh³⁺), palladium ions (e.g., Pd²⁺), platinum ions (e.g., Pt³⁺), gold ions (e.g., Au³⁺), silver ions (e.g., Ag⁺), and the like. In some embodiments, the metal ions are provided as a soluble salt of an alkaline earth metal. Alkaline earth metal ions include, for example, magnesium ions (e.g., Mg²⁺), calcium ions (e.g., Ca²⁺), strontium ions (e.g., Sr²⁺), barium ions (e.g., Ba²⁺), and the like. In some embodiments, the metal ions are provided as a soluble salt of an alkali metal. Examples of alkali metal ions include, for example, lithium ions (e.g., Li⁺), sodium ions (e.g., Na⁺), potassium ions (e.g., K⁺), and the like. In some embodiments, the metal ions are selected from Ca ions, Zn ions, Fe ions, Co ions, Mg ions, Ni ions, Ag ions, Au ions, Cu ions, Mn ions, and combinations thereof. In some embodiments, the ions are calcium ions (i.e., Ca²⁺ ions). Metal ions are generally provided as soluble salts in the dope solutions of the invention. For example, calcium cations can be provided in the dope solution as calcium chloride (CaCl₂) or calcium bicarbonate (Ca(HCO₃)₂). As another non-limiting example, lithium cations can be provided in the dope solution as lithium carbonate (Li₂CO₃).

The dope solution in the methods of the invention can include any suitable amount of polymer. It will be appreciated that the amount of polymer may be modified, for example for optimization of the electrospinning process. For example, the dope solution may comprise 2-30% w/v polymer. For a dope solution comprising chitosan or PVA as the polymer, the dope solution may comprise less than 5% w/v polymer.

In some embodiments, the dope solution contains gelatin in an amount ranging from about 5-15% w/v, e.g., 5-10% w/v gelatin. In some embodiments, the dope solution contains 1, 2, 3, 4, 5, 6, 7, 8, 9, 10, 11, 12, 13, 14, 15, 16, 17, 18, 19, or 20% w/v gelatin. In some such embodiments, the dope solution further comprises TFE.

In some embodiments, the dope solution contains collagen in an amount ranging from about 5-15% w/v, e.g., 5-10% w/v collagen. In some embodiments, the dope solution contains 1, 2, 3, 4, 5, 6, 7, 8, 9, 10, 11, 12, 13, 14, 15, 16, 17, 18, 19, or 20% w/v collagen. In some such embodiments, the dope solution further comprises HFIP. In some such embodiments, the dope solution further comprises water (e.g., around 1-20% w/v water, or around 1-10% w/v water).

The type of collagen is not limited to any particular type of collagen. For example, collagen types I, II, III, IV, V, VI, VII, VIII, VIX, or X, etc. can be used herein. The collagen can be recombinant or naturally occurring collagen. In some embodiments, the collagen can be vertebrate collagen. In some embodiments, the collagen is mammalian collagen such as, for example, human collagen. The type of collagen that is used can be varied depending upon how the polymer product is intended to be used. For example, when osteoclasts or osteoclast precursors are to be cultured using the polymer products, type I collagen can be used. Sources of type I collagen include rat tail collagen, bovine dermis collagen, and human placental collagen.

The dope solution may include any suitable amount of cross-linking agent, such as a catecholamine, relative to the amount of polymer. It will be appreciated that the amount of cross-linking agent, such as a catecholamine, may be modified to achieve a desired level of cross-linking in the polymer product. For example, the amount of cross-linking agent is less than the amount of polymer in the dope solution.

Typically, a cross-linking agent will be present in the dope solution in an amount ranging from 0.1% to about 20% w/w, with respect to the amount of the polymer. The concentration of the cross-linking agent (e.g., a catecholamine or a polyphenol) in the dope solution can range, for example, from about 0.1% to about 0.25% w/w, or from about 0.25% to about 0.5% w/w, or from about 0.25% to about 0.75% w/w, or from about 0.75% to about 1% w/w, or from about 1% to about 2.5% w/w, or from about 2.5% to about 5% w/w, or from about 5% to about 7.5% w/w, or from about 7.5% to about 10% w/w, or from about 10% to about 12.5% w/w, or from about 12.5% to about 15% w/w, or from about 15% to about 17.5% w/w, or from about 17.5% to about 20% w/w. The concentration of the cross-linking agent (e.g., a catecholamine or a polyphenol) in the dope solution can range from about 5% to about 15% w/w, or from about 1% to about 30% w/w. In some embodiments, the concentration of the cross-linking agent in the dope solution is around 10% w/w, with respect to the amount of the polymer. In some such embodiments, the dope solution comprises dopamine in an amount of about 10% w/w. In some such embodiments, the dope solution comprises norepinephrine in an amount of about 10% w/w.

In some embodiments, the amount of cross-linking agent is about 1-10 w/w of the polymer in the dope solution. In some embodiments, the amount of catecholamine is about 1-10 w/w of the polymer in the dope solution.

Further still, the dope solution may include any suitable amount of polyhydroxy antimicrobial agent and/or polyamine antimicrobial agent relative to the amount of polymer. It will be appreciated that the amount of polyhydroxy antimicrobial agent and/or polyamine antimicrobial agent may be modified to achieve a desired level of polyhydroxy antimicrobial agent and/or polyamine antimicrobial agent in the polymer product. For example, the amount of polyhydroxy antimicrobial agent and/or polyamine antimicrobial agent is less than the amount of polymer in the dope solution.

Typically, an antimicrobial agent will be present in the dope solution in an amount ranging from 0.01% to about 10% w/v. The concentration of the antimicrobial agent (e.g., a polyhydroxy or polyamine antimicrobial agent) in the dope solution can range, for example, from about 0.01% to about 0.05%, or from about 0.05% to about 0.1%, or from about 0.1% to about 0.5%, or from about 0.5% to about 1%, or from about 1% to about 5%, or from about 5% to about 10%. The concentration of the antimicrobial agent (e.g., a polyhydroxy or polyamine antimicrobial agent) in the dope solution can range from about 0.25% to about 2.5%, or from about 0.1% to about 5%, or from about 0.05% to about 10%. In some embodiments, the concentration of the antimicrobial agent in the dope solution is around 0.5% w/v. In some such embodiments, the dope solution comprises amphotericin B, caspofungin, vancomycin, polymyxin B, or daptomycin in an amount of about 0.5% w/v.

In certain embodiments, the amount of polyhydroxy antimicrobial agent and/or polyamine antimicrobial agent may be about 0.1-10% w/w of the polymer in the dope solution.

The dope solution can include any suitable amount of metal ions. Typically, a metal ion will be present in the dope solution in an amount ranging from 0.1 mM to about 100 mM, depending on the solubility of the metal salt in the solvent mixture. The concentration of the metal ion (e.g., Ca²⁺ or Li⁺) in the dope solution can range, for example, from about 0.1 mM to about 0.25 mM, or from about 0.25 mM to about 0.5 mM, or from about 0.25 mM to about 0.75 mM, or from about 0.75 mM to about 1 mM, or from about 1 mM to about 25 mM, or from about 25 mM to about 50 mM, or from about 50 mM to about 75 mM, or from about 75 mM to about 100 mM. The concentration of the metal ion (e.g., Ca²⁺ or Li⁺) in the dope solution can range from about 15 mM to about 25 mM, or from about 10 mM to about 40 mM, or from about 5 mM to about 50 mM. In some embodiments, the concentration of the metal ion in the dope solution is around 20 mM. In some such embodiments, the dope solution comprises calcium chloride in an amount of about 20 mM.

As described in more detail below, the methods of the invention generally include the use of an external electric field for atomization of the polymeric dope solution during the spinning process. When the external electrostatic field is applied to the dope solution, a suspended conical droplet is formed at the solution source (e.g., a needle used for injection of the dope solution into the spinning apparatus). Initially, the surface tension of the droplet is in equilibrium with the electric field. Electrostatic atomization occurs when the electrostatic field is strong enough to overcome the surface tension of the liquid. The liquid droplet then becomes unstable and a tiny jet is ejected from the surface of the droplet. The material eventually reaches a grounded target, where it is collected as an interconnected web containing fine fibers.

Any suitable electric field can be used in the methods of the invention. Typically, electric fields ranging from around 100 V to around 100 kV are used in the methods of the invention. The electric field can range, for example, from 500 V to 50 kV, or from 1 kV to 25 kV, or from 5 kV to 15 kV. The electric field can be 5 kV, 5.5 kV, 6 kV, 6.5 kV, 7 kV, 7.5 kV, 8 kV, 8.5 kV, 9 kV, 9.5 kV, 10 kV, 10.5 kV, 11 kV, 11.5 kV, 12 kV, 12.5 kV, 13 kV, 13.5 kV, 14 kV, or 15 kV. Other field strengths can be used depending on the composition of the particular dope solution used for the electrospinning process.

Any suitable flow rate can be used for introducing the dope solution from the source into the electric field. Typically, the flow rate will range from about 0.1 mL/hr to about 5 mL/hr. The flow rate can range, for example from 0.1 mL/hr to 0.5 mL/hr, or from 0.5 mL/hr to 1 mL/hr, or from 1 mL/hr to 1.5 mL/hr. The flow rate can range from 0.5 mL/hr to 1.5 mL/hr, or from 0.5 mL/hr to 1 mL/hr. The flow rate can be about 0.1, 0.2, 0.3, 0.4, 0.5, 0.6, 0.7, 0.8, 0.9, 1.0, 1.1, 1.2, 1.3, 1.4, or 1.5 mL/hr. Other flow rates can be used depending on factors including the composition of the dope solution and the strength of the electric field used in the process.

The target surface used for fiber collection can be placed at any suitable position with respect to the source of the dope solution. The distance between the dope solution source and the target surface will typically range from about 5 cm to 50 cm. The distance can range, for example, from 5 to 10 cm, or from 10 cm to 15 cm, or from 15 cm to 20 cm, or from 20 cm to 25 cm. The distance can range from 5 cm to 30 cm, or from 10 cm to 20 cm. The distance between the dope solution source and the target surface can be around 5, 6, 7, 8, 9, 10, 11, 12, 13, 14, or 15 cm. Other distances can be employed depending on factors including the composition of the dope solution, the strength of the electric field, and the dope solution flow rate used in the process.

The present invention provides polymer products obtainable by a method according to any one aspect of the invention. In general, the polymer products are hydrophilic, water-soluble, and/or biocompatible. In some embodiments, the polymer product comprises polymeric fibers with cross-links of a polyphenolic compound, a catecholamine compound, a polymeric catecholamine compound or a combination thereof. In some embodiments, the polymer product comprises polymeric fibers with cross-links of a polyhydroxy antimicrobial agent, a polyamine antimicrobial agent, a catecholamine compound, a polymeric catecholamine compound or a combination thereof. In some embodiments, the polymer products further include metal ions as described above.

The polymer products of the invention include any product of any shape or size fabricated from the electrospun polymeric fibers as desired. The polymeric fibers of the polymer product may be nanofibers or microfibers. In certain embodiments, the polymer product comprises a fiber mat.

In some embodiments, the polymer product comprises a substrate for cell and/or tissue culture. The polymer products can be incorporated in articles for cell culture, such as a Petri dish, a multi-well plate, a flask, a slide, a beaker or other container having a well. Other examples of cell culture articles include, for example, a 384-well microplate, a 96-well microplate, a 24-well dish, an 8-well dish, a 10 cm dish, and a T75 flask. Such polymer products can be used for the culture of cells such as osteoclasts and tissues such as bone tissue.

In some embodiments, the polymer product is used as a wound dressing or as a component of a wound dressing. The polymer product can be formed on, or otherwise integrated with, a solid, semi-solid, or liquid wound dressing carrier material suitable for administration to a human or other animal. Wound dressing carriers are typically characterized by high purity levels and low toxicity levels, which levels are sufficient to render them suitable for administration to the human or animal being treated. The wound dressing can be in any form such as a pad, gauze, cloth, sheet, or the like. The dressing can be used by itself or in conjunction with a medicinal or other substance applied thereto or contained therein, and can comprise one or more layers.

The wound dressing can comprise one or more layers of absorbent and/or wicking materials capable of being employed in wound dressings to receive proteinaceous exudate from a wound. The wound dressing can comprise woven or non-woven cotton, gauze, a polymeric net or mesh such as polyethylene, nylon, polypropylene, or polyester, an elastomer such as polyurethane or polybutadiene elastomers, or a foam such as open cell polyurethane foam. When the wound dressing includes a layer of a nonwoven fabric, such non-woven can be a spun-bonded or spun-laced construction. Further, wet-laid and air-laid non-woven fabrics can be employed. The wound dressing can contain, for example, spun-bonded polyester staple fiber fabric or non-woven cellulose acetate.

In addition, the wound dressing can further include a hydrophilic material capable of retaining its integrity even after absorbing 2 to 20 times its weight of exudate. Such hydrophilic materials include, but are not limited to, sodium carboxymethylcellulose, various polyacrylamide, polyacrylonitrile and acrylic acid polymers, Karaya gum, and polysaccharides. Acrylics and acrylates, which are unsubstituted or variously substituted, can be employed in the absorbent layer.

Accordingly, some embodiments of the invention provide a method for preparing a polymer product comprising
i) electrospinning from a dope solution comprising at least one polymer and at least one cross-linking agent to prepare the polymer product,
wherein the cross-linking agent comprises at least one catecholamine, and wherein the electrospun fibers can be collected on a bare metallic collector or on a non-woven fabrics such as bandage gauze, and
ii) exposing the polymer product to at least one gaseous alkaline reagent.

The polymer products described herein can further comprise one or more bioactive agents that can facilitate cell adhesion to the microfibers, promote cell function, promote cell growth, or modulate other cell and tissue functions. Bioactive agents can be physically adsorbed on a polymer product such as a fiber mat, or the bioactive agents can be covalently bonded to the polymer product using a chemical crosslinker. These bioactive agents stimulate cell growth, migration of differentiated and non-differentiated cells, and the differentiation of non-differentiated cells (e.g., progenitor and stem cells) towards and at the repair, regeneration or new growth site. Progenitor cells that are typically involved include endothelial progenitor cells (EPCs) and mesenchymal progenitor cells (MPCs). Suitable bioactive agents for inclusion in the polymer products include growth factors and differentiation factors that stimulate cell growth and differentiation of the progenitor and stem cells.

Suitable growth factors and cytokines include, but are not limited to stem cell factor (SCF), granulocyte-colony stimulating factor (G-CSF), granulocyte-macrophage stimulating factor (GM-CSF), stromal cell-derived factor-1, steel factor, vascular endothelial growth factor (VEGF), transforming growth factor-beta (TGFβ), platelet derived growth factor (PDGF), angiopoietins (Ang), epidermal growth factor (EGF), bone morphogenic protein (BMP), fibroblast growth factor (FGF), hepatocyte growth factor, insulin-like growth factor (IGF-1), interleukin (IL)-3, IL-1α, IL-1β, IL-6, IL-7, IL-8, IL-11, and IL-13, colony-stimulating factors, thrombopoietin, erythropoietin, fit3-ligand, and tumor necrosis factor α. (TNFα).

Examples of growth factors include EGF, bFGF, HNF, NGF, PDGF, IGF-1 and TGFβ. These growth factors can be mixed with the scaffold materials comprising the compositions. The bioactive agents can also have pro-angiogenic activities, e.g., VEGF, PDGF, prominin-1 polypeptide, and variants thereof that have pro-angiogenic activities, i.e., promote neovascularization and angiogenesis. The bioactive agents can promote or stimulate bone growth. For example, the bioactive agents can be bone morphogenic proteins (BMPs), which exhibit pro-osteogenic properties.

Having now generally described the invention, the same will be more readily understood through reference to the following examples which are provided by way of illustration, and are not intended to be limiting of the present invention.

### IV. Examples

### Example 1. Materials and Methods

All chemicals are of analytical grade and have been used without further purification.

### Preparation of Dope Solutions

A clear homogenous gelatin (from Porcine Skin, Type A, 300 g Bloom) solution of a desired concentration (for example 10% w/v) was prepared by dissolving the gelatin in 2,2,2-trifluoroethanol (TFE), (Sigma-Aldrich) at room temperature for 24 hours with continuous stirring.

Collagen (purified bovine dermal collagen type I) solution of a desired concentration (for example 8% w/v) was prepared by dissolving the collagen in hexafluoroisopropanol (HFIP), (Sigma-Aldrich) at room temperature for 24 hours with continuous stirring.

The amount of catecholamine (dopamine, norepinephrine or α-methyl norepinephrine from Sigma-Aldrich) is added as desired and may be for example, from 1-10% (w/w of the polymer) in the final dope solution.

### Electrospinning

A custom-built electrospinning device (comprising of a Gamma High Voltage DC power source, USA) and a syringe pump (KDS 100, KD Scientific, Holliston, Mass.) was used for the fabrication of electrospun polymer fiber.

Dope solution prepared was fed at the rate of 0.8 ml/h into a 5 ml standard plastic syringe (made of polypropylene) attached to a 27 G blunted stainless steel needle using the syringe pump. Droplets would form at the orifice of the needle, and these droplets were stretched and splayed into continuous long fibers by applying a high voltage of 11.5 KV. The fibers were collected on grounded targets of different substrates (e.g. aluminum foil, glass cover slips etc.) placed at a distance of approximately 12 cm from the needle.

All electrospinning was performed at room temperature with an air humidity of approximately 25%.

### Preparation of Electrospun Polycatecholamine-Coated Gelatin or Collagen Fibers by Ammonia Diffusion Method (AMD)

Electrospun dopamine-coated gelatin or collagen fiber mats were prepared by electrospinning a dope solution containing defined gelatin or collagen content (2% to 30% w/v) with varying concentrations (1% to 10% w/w of gelatin or collagen in the dope solution) of dopamine (Sigma-Aldrich).

After 48 hours of vacuum drying to remove residual solvents from the electrospun dopamine-coated gelatin or collagen fiber mats, the fiber mats were exposed to a suitable amount (for example 5 g) of ammonium carbonate powder (Sigma-Aldrich) in a sealed desiccator for approximately 24 hours.

### Preparation of Dopamine-Coated Gelatin or Collagen Fibers by Tris-HCl Method

Electrospun gelatin or collagen fibers were immersed in a solution of 20 mg/ml dopamine hydrochloride (Sigma-Aldrich) at pH 8.5 for 5 hours. After coating, the polydopamine-coated gelatin or collagen fibers were rinsed with distilled water and freeze dried.

### Scanning Electron Microscope and Transmission Electron Microscope

Field Emission Scanning Electron Microscope (FEI-QUANTA 200F, the Netherlands) was utilized to analyze the morphology of electrospun fibers, and to investigate the effect different dopamine concentrations have on gelatin/collagen fiber diameter and their surface characteristics. All the SEM measurements were carried out at an accelerating voltage of 15 KV after sputter coating the samples with Platinum (JEOL JSC-1200 fine coater, Japan). SEM images for different electrospun gelatin/collagen fibers samples were then analyzed using Image Analysis Software (Image J, National Institute of Health, USA) to calculate the average fiber diameter. The diameter of approximately 100 randomly selected nanofibers was measured to obtain the average nanofiber diameter in each gelatin/collagen fibers sample. Transmission electron microscopy of different electrospun gelatin/collagen fibers samples has been performed using JEOL JEM-3010 instrument.

### Fiber Mechanical Properties

Tensile testing of the electrospun fibers was performed using tabletop tensile Tester (Instron 5345, USA) using a load cell of 10 N capacity at ambient conditions. Rectangular specimens of 10×20 mm dimensions were cut (using a specimen frame) and peeled off from the nanofiber sheet spun on an aluminum foil, and tested at a cross-head speed of 5 mm min⁻¹. Five samples were tested for each type of electrospun fiber or fiber mat during this study. All the mechanical characteristics (Tensile strength, Failure Strain, Young's Modulus and Work of Failure) were calculated based on the generated stress-strain curves for each type of electrospun fiber or fiber mat. Before testing, the samples were vacuum-dried for 48 hours and then allowed to rest for one week in 65% relative humidity.

### Attenuated Total Reflectance-Fourier Transform Infrared (ATR-FTIR) Spectroscopy

ATR-FTIR spectra of all the electrospun fiber mats were analyzed with AVATAR 380 FTIR spectrometer (Thermo Electron, Waltham, Mass.), over a range of 400-4000 cm⁻¹. Spectra was recorded as the average of 200 scans at 1 cm⁻¹ resolution at 25° C. using empty accessory as the blank.

### Reverse Phase High-Pressure Liquid Chromatography (RP-HPLC)

To identify the differences in the composition of polydopamine formed using two different methods, namely the ammonium carbonate diffusion method and the Tris-HCl method, RP-HPLC was performed on the polydopamine prepared by the ammonium carbonate diffusion method and Tris-HCl method respectively. RP-HPLC was carried out using Waters HPLC analyzer with C18 analytical column (Phenomenex, Calif., USA). The programming of the mobile phases (A=distilled water with 1% trifluoroacetic acid, B=acetonitrile with 1% trifluoroacetic acid) were as follows: a static mode of 95:5 (A:B, v:v) from 0-5 min, a gradient mode of 10:90 from 5-35 min, a gradient mode of 5:95 from 35-40 min, a gradient mode of 95:5 from 40-43 min and static mode of 95:5 from 43-50 min. A UV-Visible detector was used and the detector wavelength was 280 nm (100 μl sample injection).

### Contact Angle Measurement

The hydrophilic/hydrophobic properties of electrospun fiber mats were measured by sessile drop water contact angle measurement using a VCA Optima Surface Analysis system (AST products, Billerica, Mass.). Distilled water was used for drop formation.

### Cell Culture and Treatments

Human dermal fibroblast cells were cultured according to the procedures recited in Biomacromolecules, 2005, 6 (5), pp 2583-2589. Briefly, cells were cultured in DMEM medium (Gibco®) supplemented with 10% (v/v) fetal bovine serum, 50 U/ml penicillin and 50 μg/ml streptomycin in a humidified incubator at 37° C. and 5% CO₂. All the cell culture reagents were obtained from Life Technologies Corporation (Singapore). For biocompatibility experiments, cells were seeded onto the coverslips precoated with scaffolds and placed at the bottom of the 12-well plates (Nunc®) at a density of 10×10⁴ cells/well and allowed to grow for 24 hours before analysis. For the negative control well(s), nocodazole was added to a final concentration of 5 μg/mL in the culture medium.

### Confocal Fluorescent Microscopy

For confocal microscopy, dermal fibroblast cells were cultured on scaffold-coated glass cover-slips for 24 hours and then cells were fixed in 3% paraformaldehyde. After washing with phosphate buffered saline (PBS), cells were stained and fluorescently labeled with FITC conjugated α-tubulin (Sigma-Aldrich) and Alexa Fluor 569 phalloidin (Molecular Probes®) to visualize the cytoskeletal systems, and Hoechst (Sigma-Aldrich) to visualize nuclei. Coverslips were mounted on glass slides using Flouromount™ Aqueous mounting (Sigma-Aldrich). Confocal imaging was carried out by a laser scanning microscope (Zeiss LSM710-Meta, Carl Zeiss Microimaging Inc., NY, USA) using a ×40 oil immersion objective lens. Excitation wavelengths used were 405 nm, 488 nm and 561 nm, and emission filters were BP 420-480 nm, BP 505-530 nm and 572-754 nm respectively. At least 20 different microscopic fields were analyzed for each sample.

Fluorescence spectral (λ-scan) scanning was performed in triplicate for the electrospun gelatin fiber, and each dopamine-loaded gelatin fibers after exposure to gaseous ammonia. All samples were scanned in the emission range of 400-600 nm in 10 nm increments using Synergy™ H1 Microplate reader (BioTek Instruments, Inc., Winooski, Vt., USA) at a fixed excitation of 360 nm. Data was collected using Gen5™ Data Analysis Software (BioTek Instruments, Inc., Winooski, Vt., USA). The surface area of the well was completely covered with the polydopamine-coated samples and the read height for the measurement was adjusted to 4.8 mm. The values were subtracted for background fluorescence using scanning blank wells.

### MTS-Based Cell Viability Assay

Cell viability was determined using CellTier 96® Aqueous One solution cell proliferation assay kit according to the manufacturer's instruction (Promega Corporation, Madison, Wis.), and according to the procedures recited in Kim B J et al., Reinforced multifunctionalized nanofibrous scaffolds using mussel adhesive proteins, Angew Chem Int Ed Eng, 2012; 51:675-8. This assay evaluates mitochondrial function by measuring the ability of viable cells to reduce MTS ((3-(4,5-dimethylthiazol-2-yl)-5-(3-carboxymethoxyphenyl)-2-(4-sulfophenyl)-2H-tetrazolium)) into a quantifiable blue, insoluble formazon product. Briefly, at the end of the treatment period, cells growing on the scaffold-coated coverslips placed in a 12-well plate containing 500 μL of cell culture medium were incubated with 50 μL of MTS tetrazolium solution (provided by manufacturer) for 2 hours at 37° C. Subsequently, the absorbance was measured at 490 nm using a microplate reader (Infinite M200 Pro, Tecan, Mannedorf, Switzerland) and then relative cell viability was calculated. Each treatment was performed in triplicate.

### Statistical Analysis

The data was expressed as mean±standard error of mean. For comparison of two groups, p-values were calculated by two-tailed unpaired student's t-test. In all cases p-values ≤0.05 was considered to be statistically significant.

### Example 2. Catecholamine as the Cross-Linking Agent

### Scanning Electron Microscopy Results of Fiber Mats

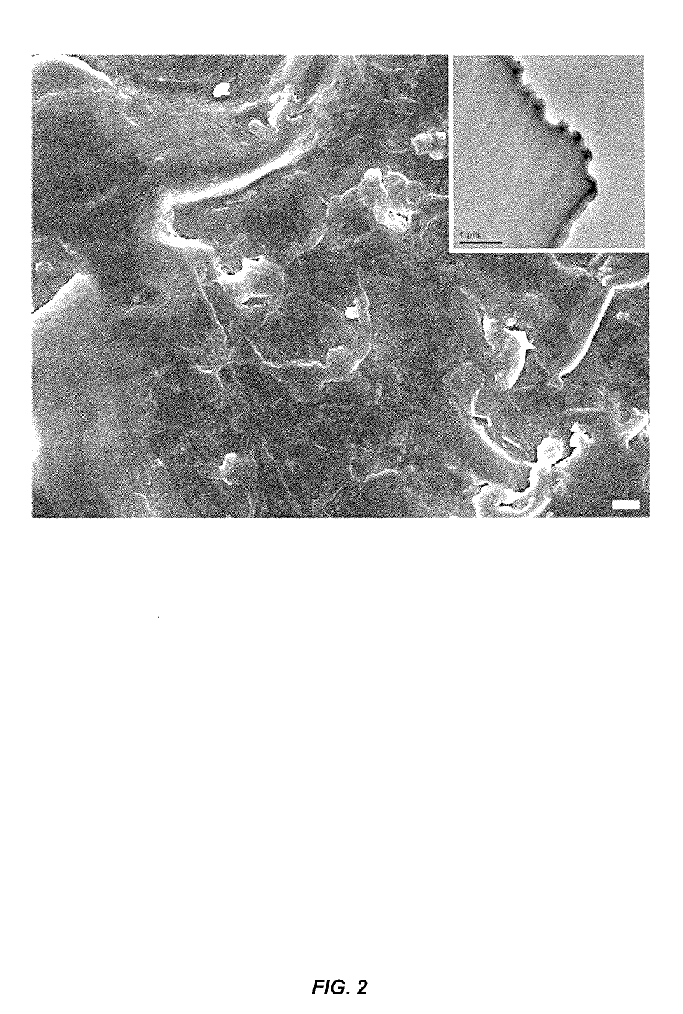

Scanning electron microscopy images of polydopamine coated electrospun gelatin fiber mats prepared by the Tris-HCl method shows that the fiber mat has a rough surface and the coating completely obscures the fibrous morphology of the fiber mat (FIG. 2). This result is consistent with reports that the composition of polydopamine coating prepared by the Tris-HCl method is complex and contained a mixture of oligomeric species (Dreyer et al., 2012; Vacchia et al., 2013) and this high aggregation tendency of oligomers and the heterogeneity of coating are responsible for the observed surface roughness in polydopamine coating (Hong et al., 2011 and 2013).

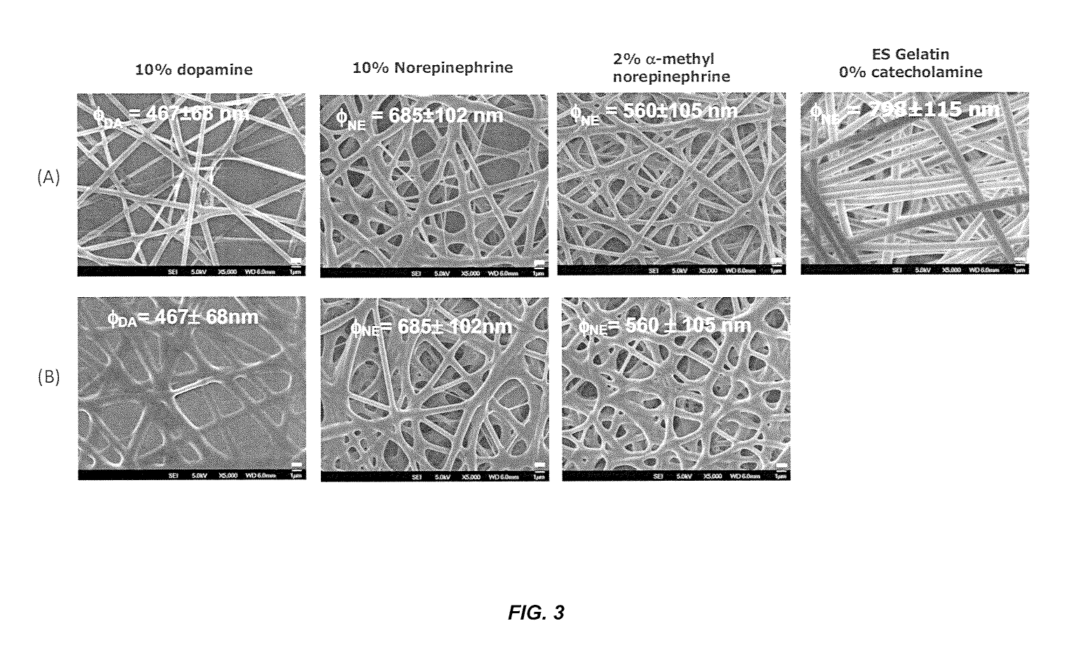

In contrast, scanning electron microscopy images of various catecholamine-coated gelatin fiber mats prepared from electrospinning a dope solution with dopamine and different catecholamines before and after exposure to gaseous ammonia and carbon dioxide from ammonium carbonate shows that the macroporous architecture of the fiber mat was maintained even after exposure to gaseous ammonia and carbon dioxide from ammonium carbonate (FIG. 3).

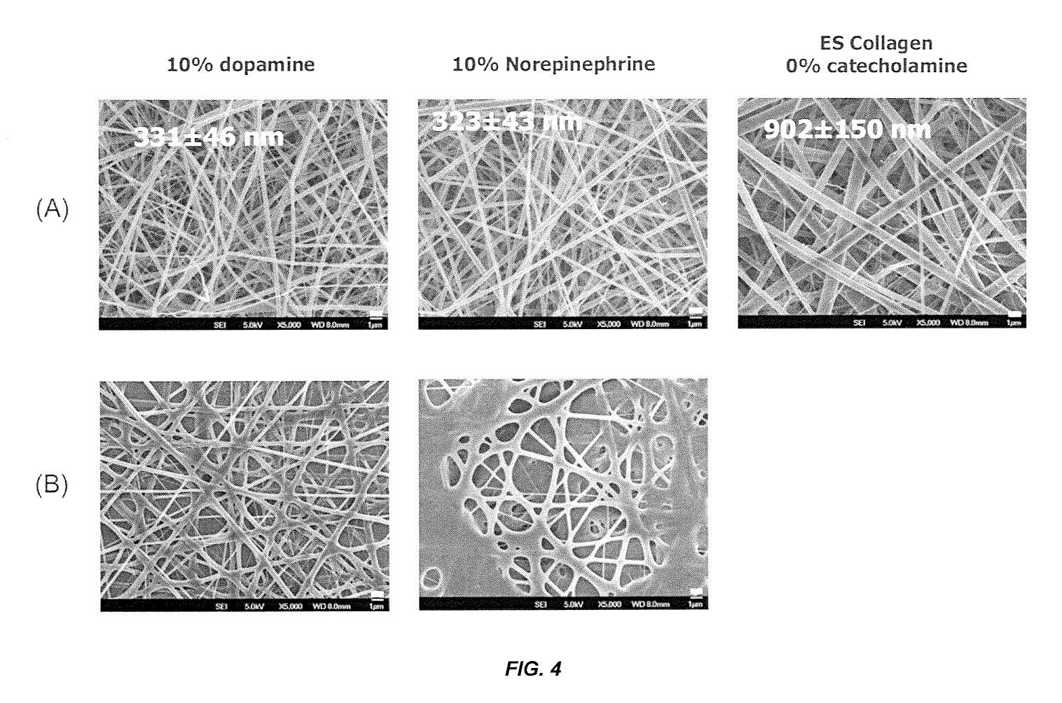

A similar result was observed for catecholamine-coated collagen fiber mats prepared from electrospinning a dope solution with collagen and different catecholamines before and after exposure to gaseous ammonia and carbon dioxide form ammonium carbonate (FIG. 4).

In both cases, the coating occurred across the entire length of the electrospun gelatin and collagen fibers after exposure to gaseous ammonia and carbon dioxide from ammonium carbonate.

When compared to the Tris-HCl or alkaline solution method of preparing polydopamine coated electrospun polycaprolactone (PCL), poly-L-lactic acid (PLA), poly(L-lactide-co-ε-caprolactone) (PLCL) and PCL-gelatin blend nanofibers, the ammonium carbonate diffusion method produced a relatively smooth film or surface of polydopamine coating on the electrospun gelatin nanofibers (cf. references in Table I).

TABLE I
Properties of polydopamine electrospun fibers
prepared by alkaline solution routes

| Method of | | Mechanical | | |
|---|---|---|---|---|
| Coating | Polymers used | Properties | Disadvantages | Reference |
| Polydopamine | PVA | NA | Swelling and | Son et al., |
| coating | | | dissolution of the | 2013 |
| followed by | | | polymers and | |
| silver | | | formation of | |
| deposition | | | polydopamine | |
| | | | aggregates on the | |
| | | | surface. | |
| Polydopamine | PCL-Gelatin | NA | Formation of pDA | Ku et al., 2010 |
| coating | blends | | aggregates | |
| Polydopamine | Poly(L-lactic | NA | Formation of | Rim et al., |
| coating | acid) | | aggregates | 2012 |
| polydopamine | poly(L-lactide- | Increased | Formation of | Shin et al., |
| | co-ε- | Young's | aggregates | 2011 |
| | caprolactone | modulus | | |
| Polydopamine | PCL | Increase | Rough coating and | Xie et al., 2012 |
| | | Young's | microcracks at | |
| | | modulus | higher [dopamine] | |
| | | | and longer | |
| | | | crosslinking time | |

Physical and/or Mechanical Properties

Increases in the key physical and/or mechanical properties such as ultimate tensile strength, Young's modulus and toughness were observed in the polydopamine-coated polymer fiber mats after exposure to gaseous ammonium and carbon dioxide when compared to electrospun polymer fiber mats or electrospun dopamine-coated polymer fiber mats not exposed to gaseous ammonia (Table II). It is also noted from Table II that electrospun dopamine-coated polymer fiber mats not exposed to gaseous ammonia also showed increases in these physical and/or mechanical properties compared to the electrospun polymer fiber mats.

TABLE II
Mechanical properties of electrospun fibers and dopamine coated electrospun fibers
before, and after exposure to ammonia and carbon dioxide derived from ammonium
carbonate

| | Tensile | | Young's | Work of |
|---|---|---|---|---|
| | Strength | Failure | Modulus | Failure |
| Sample | (MPa) | Strain (%) | (MPa) | (MJ/m³) |
| Electrospun Gelatin | 2.7 ± 0.4 | 9.0 ± 1.3 | 103 ± 5.5 | 0.19 ± 0.04 |
| Electrospun Gelatin + 1% w/w | 2.8 ± 0.3 | 10.9 ± 1.1 | 59 ± 3.8 | 0.25 ± 0.04 |
| dopamine of | | | | |
| gelatin in dope solution | | | | |
| Electrospun Gelatin + 2% w/w | 4.6 ± 0.6 | 7.5 ± 1 | 95 ± 3.8 | 0.21 ± 0.05 |
| dopamine of | | | | |
| gelatin in dope solution | | | | |
| Electrospun Gelatin + 1% w/w | 3.9 ± 0.2 | 14.9 ± 1.3 | 71 ± 4 | 0.46 ± 0.03 |
| dopamine of | | | | |
| gelatin in dope solution | | | | |
| (using ammonia | | | | |
| diffusion method) | | | | |
| Electrospun Gelatin + 1% w/w | 5.0 ± 0.2 | 10.9 ± 0.5 | 99 ± 5 | 0.4 ± 0.03 |
| dopamine of | | | | |
| gelatin in dope solution | | | | |
| (using ammonia | | | | |
| diffusion method) | | | | |
| Electrospun Collagen | 4.9 ± 0.5 | 6.0 ± 1.3 | 156.7 ± 29 | 0.22 ± 0.07 |
| Electrospun Collagen + | 5.3 ± 1.2 | 5.8 ± 1.6 | 108.8 ± 27.3 | 0.33 ± 0.25 |
| 10% w/w dopamine of | | | | |
| collagen in dope solution | | | | |
| Electrospun Collagen + | 12.4 | 4.4 | 267.6 | 0.314 |
| 10% w/w dopamine of | | | | |
| collagen in dope solution | | | | |
| (using ammonia | | | | |
| diffusion method) | | | | |

### Characterization of Electrospun Gelatin by ATR-FTIR

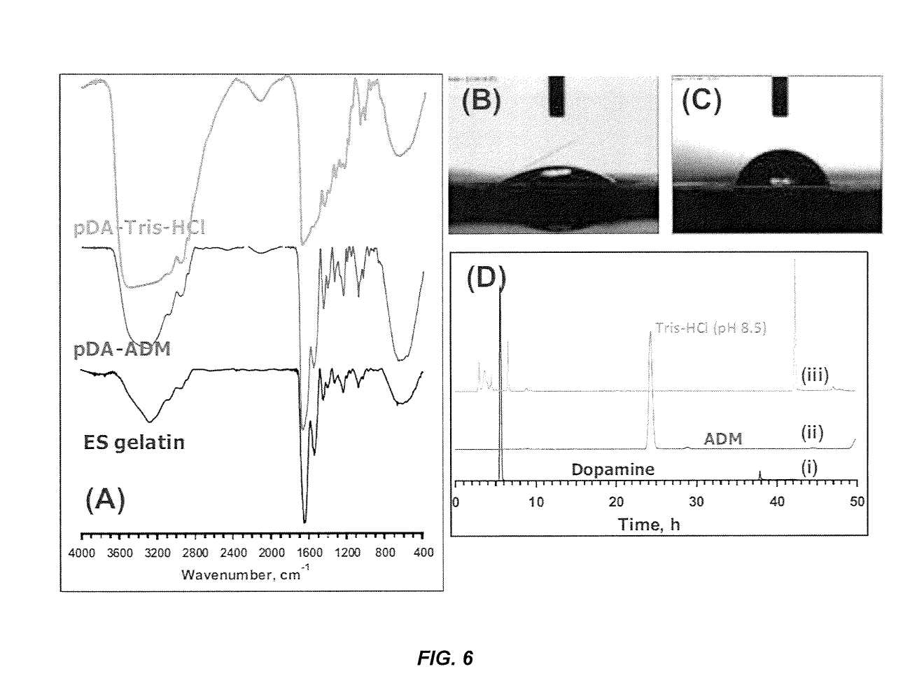

The electrospun polydopamine-coated gelatin nanofibers were investigated using attenuated total reflectance Fourier transform infrared spectroscopy (ATR-FTIR). FIG. 6(A) shows the ATR-FTIR of electrospun gelatin fibers, electrospun polydopamine-coated gelatin fibers prepared by the ammonia diffusion method, and polydopamine-coated gelatin fibers prepared by the Tris-HCl method.

The ATR-FTIR of electrospun gelatin fiber mats displayed absorption peaks around 1640 cm-1, 1540 cm⁻¹ and 1240 cm⁻¹ which are assigned to amide I (C═O Stretching), amide II (N—H bending & N—H stretching) and amide III (C—N stretching & N—H in-phase bending) peaks, respectively. A broad band centered at 3300 cm₋₁, as seen in FIG. 6(A), is assigned to the combination of Amide A and Amide B peaks which originated due to N—H stretching. The polydopamine-coated gelatin fiber mats prepared by the ammonia diffusion method resulted in significant broadening, and increase in intensity of the bands spanning the range of 3200-3500 cm⁻¹, thereby indicating the presence of additional hydroxyl functionalities due to the presence of polydopamine.

It is interesting to note that all the intrinsic spectroscopic features of gelatin were still observed in the electrospun dopamine-coated gelatin fiber mats exposed to gaseous ammonia and carbon dioxide from ammonium carbonate. However, for the polydopamine-coated gelatin fiber mats prepared by the Tris-HCl method, peaks of gelatin were obscured as a result of polydopamine coating. The indole or indoline structures (1605 cm⁻¹ and 1515 cm⁻¹) observed after dopamine polymerization overlapped with amide I and amide II peaks of gelatin, resulting in the intense broad bands in this region.

### RP-HPLC to Assess the Composition of Polydopamine-Coated Gelatin Fiber Mats Prepared by the Tris-HCl Method and Ammonia Diffusion Method

To identify the differences in composition of polydopamine prepared using the Tris-HCl method and the ammonia carbonate diffusion method, reverse-phase chromatography (RP-HPLC) was performed on:

(i) dopamine monomer;

(ii) electrospun polydopamine-coated gelatin fiber mats prepared by the ADM; and

(iii) polydopamine-coated gelatin fiber mats prepared by Tris-HCl method.

The results obtained were then compared. As seen from (ii) of FIG. 6(D), the monomer peak of dopamine (at ˜6 min) was barely visible for the electrospun polydopamine-coated gelatin fiber mats prepared by the ADM, and only a single peak at ˜25 min was observed, indicating complete conversion of dopamine monomer and homogenous polymerization of dopamine. For the solution based Tris-HCl polymerization, however, a number of peaks were observed in addition to a small amount of monomeric dopamine as seen in (iii) of FIG. 6(D). These results are consistent with reports that the composition of polydopamine coating prepared by the Tris-HCl method is complex and contained a mixture of oligomeric species (Dreyer et al., 2012; Vacchia et al., 2013); with this high aggregation tendency of oligomers and the heterogeneity of coating responsible for the observed surface roughness in polydopamine coating (Hong et al., 2011 and 2013).

On the other hand, the RP-HPLC results suggest that there is a controlled polymerization of dopamine in the gelatin fiber mat after exposure to gaseous ammonia when compared to the heterogeneous polymerization by solution route, thus promoting uniform inter-fiber fusion and smooth surface coating of cross-linking agents.

### Contact Angle Measurements

To determine the wettability (hydrophilic/hydrophobic properties) of electrospun polydopamine-coated gelatin fiber mats, water contact angle (θ_{static}) was determined. For the electrospun gelatin fiber mat and electrospun dopamine-coated gelatin fiber mat not exposed to gaseous ammonia, the water droplet was absorbed rapidly into the fiber networks, resulting in zero contact angles within 40 seconds. FIG. 6(B) shows the water drop on electrospun gelatin fiber mat at 20 seconds before its complete absorption. In comparison, for electrospun polydopamine-coated gelatin fiber mats prepared by the ammonia diffusion method, the contact angle decreased gradually and levelled off between 64.7±1.2° to 72.8±0.3° as seen in FIG. 6(C). These results indicate that polydopamine coating by ammonia diffusion method rendered the surface of gelatin more hydrophobic than electrospun gelatin fiber mats. The results are also consistent with the results reported for polydopamine coating on other substrates (Wei Q et al. 2010).

### Cell Culture Studies Including Cytotoxicity

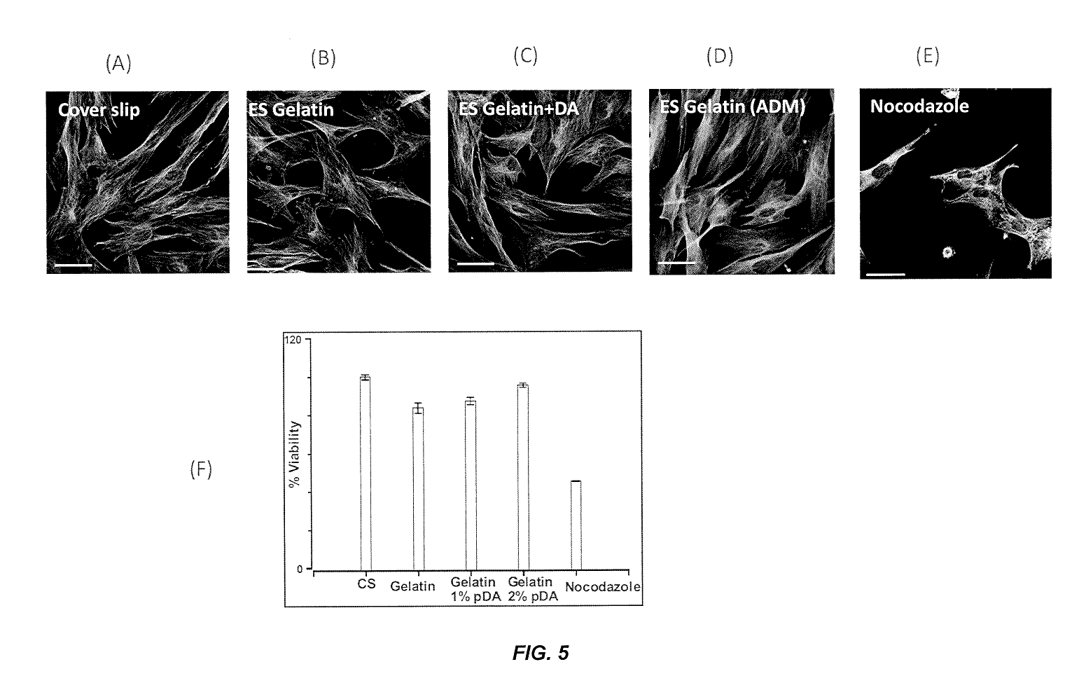

Confocal microscopy show no observable differences in the morphology and cytoskeletal architecture of dermal skin fibroblasts cells cultured on polydopamine-coated gelatin fiber mats prepared by the ADM compared to electrospun gelatin fiber mats and dopamine-coated gelatin fiber mats not exposed to gaseous ammonia [FIG. 5(B)-(D)]. No significant changes in the nuclear morphology or intensity of nuclear staining were observed in the cells grown on any one of the electrospun gelatin fiber mats when compared to cells cultured on glass coverslips (control, FIG. 5(A)). The overall shapes and sizes of cells, and nuclei were within the normal variation range, and there were no signs of significant cellular or nuclear abnormalities (e.g., condensation or blabbing), membrane bound vesicles, shrinking of the cytoplasm or cell rupture.

MTS assay further confirmed the lack of any adverse effect of polydopamine-coated gelatin fiber mats on cell viability [FIG. 5(F)].

Confocal microscopy and the MTS assay show significant cell death for the nocodazole control.

### Confocal Fluorescence Microscopy of the Fiber Mats

Confocal fluorescence microscopy imaging was conducted for electrospun gelatin fiber mats and electrospun dopamine-coated gelatin fiber mats after exposure to gaseous ammonia.

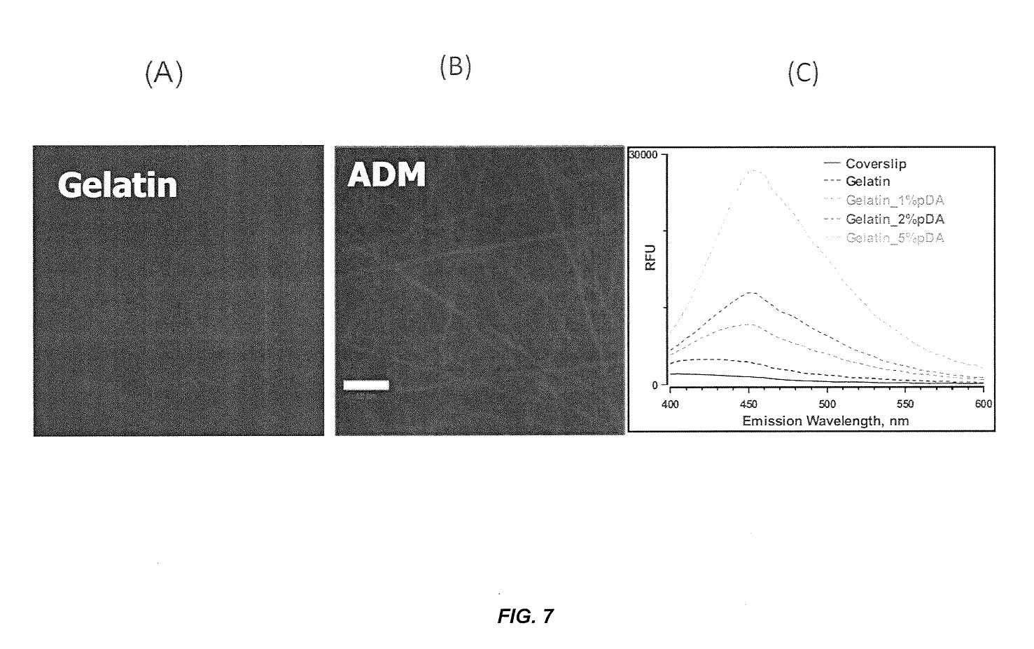

Electrospun gelatin fiber mats displayed weak fluorescence when excited at A360 nm (FIG. 7A). However, intense blue colored nanofibers were clearly visible in the electrospun polydopamine-coated gelatin fiber mats prepared by ammonia diffusion method (FIG. 7B). It was also observed that the fluorescence intensity increased with increasing concentration of dopamine present in the dope solution (FIG. 7C).

λ-scan of the electrospun gelatin fiber mats displayed a broad band around 415-440 nm and no characteristic emission maxima was observed. The electrospun dopamine-coated gelatin fiber mats, prepared by ammonia diffusion method displayed an emission maxima around 457 nm. Further, emission intensity was observed to increase with dopamine concentration, which is consistent with the microscopy results. When compared to electrospun dopamine-coated gelatin fiber mats not exposed to gaseous ammonia, the electrospun polydopamine-coated gelatin fiber mats prepared by ammonia diffusion method also showed higher fluorescence intensity, further confirming the formation of polydopamine structures.

### Summary

The data in Example 2 show that oxidative polymerization of catecholamine-coated polymer fiber mat by the ammonia diffusion method forms a smooth and non-cytotoxic (thus biocompatible) coating of polycatecholamines on the polymer fiber mat, while retaining the porous architecture of the polymer fiber mat. Moreover, compared to the polydopamine-coated polymer fiber mat prepared using the Tris-HCl method, the polydopamine-coated polymer fiber mat prepared using the ammonia diffusion method improved physical and/or mechanical properties.

### Example 3. Antimicrobial Nanofiber Polymer Products

In the areas of antimicrobial delivery and wound dressing, a high concentration of the antimicrobial at the site of infection is required to achieve clinical efficacy. Some antimicrobial delivery platforms such as polymeric and peptide hydrogels, nanoparticles, microemulsion, liposomes, niosomes, or ethnosomes can function as depots to deliver antimicrobials at the site of infections. However, these formulations generally need to be applied frequently to maintain sustained release of the drugs. Incorporation of antimicrobials into aqueous stable polymers and electrospun fiber mats have been reported (Abrigo et al., 2014; Joshi et al., 2014; Lakshiminarayanan et al., 2014; US 2014/0142025). Nevertheless, it is desirable to further improve on stability and sustained delivery of the antimicrobials over an appropriate period.

### Materials and Methods

All chemicals are of analytical grade and have been used without further purification.

### Preparation of Dope Solutions

It will be appreciated that for a different polymeric dope solution, a different suitable solvent may be used accordingly. It will also be appreciated a different polymer, a different catecholamine and a different polyhydroxy antimicrobial agent may be used to make up the dope solution. As discussed above, it will further be appreciated that different amounts of polymer, catecholamine, and antimicrobial agent may be used to make up the dope solution accordingly. Also as discussed above, each polymeric dope solution may comprise one or more polymer, one or more catecholamine and one or more polyhydroxy antimicrobial agent.

A clear homogenous gelatin (from Porcine Skin, Type A, 300 g Bloom) solution of a desired concentration (for example 10% w/v) was prepared by dissolving the gelatin in 2,2,2-trifluoroethanol (TFE), (Sigma-Aldrich). This serves as a basic dope solution for electrospinning pristine gelatin fiber mats. This serves as a basic dope solution for electrospinning pristine gelatin fiber mats.

An antimicrobial agent (amphotericin B, caspofungin, vancomycin, polymyxin B or daptomycin from Sigma Aldrich Pte Ltd, Singapore) of 0.5% w/w of the gelatin in the dope solution was added to produce separate dope solutions for electrospinning of gelatin antimicrobial fiber mats.

Dopamine (Sigma-Aldrich) was added to a concentration of 2% w/w of the polymer in the dope solution to produce separate dope solutions for electrospinning of dopamine coated gelatin antimicrobial fiber mats.

Each dope solution was kept at room temperature for 24 hours with continuous stirring before electrospinning.

### Electrospinning

A custom-built electrospinning device (comprising of a Gamma High Voltage DC power source, USA) and a syringe pump (KDS 100, KD Scientific, Holliston, Mass.) was used for the fabrication of electrospun polymer fiber.

Dope solution prepared was fed at the rate of 0.8 ml/h into a 5 ml standard plastic syringe (made of polypropylene) attached to a 27 G blunted stainless steel needle using the syringe pump. Droplets would form at the orifice of the needle, and these droplets were stretched and splayed into continuous long fibers by applying a high voltage of 11.5 KV. The fibers were collected on grounded targets of different substrates (e.g. aluminum foil, glass cover slips etc.) placed at a distance of approximately 12 cm from the needle.

All electrospinning was performed at room temperature with an air humidity of approximately 25%.

### Ammonia Diffusion Method

After 48 hours of vacuum drying to remove residual solvents, each electrospun fiber mat was then exposed to a suitable amount of ammonium carbonate powder (5 g) (Sigma-Aldrich) in a sealed desiccator for approximately 24 hours.

### Scanning Electron Microscopy (SEM)

The morphology of the fiber mats were observed with the use of a Field Emission Scanning Electron Microscope (FEI-QUANTA 200F, Oregon, USA). All the SEM measurements were carried out at an accelerating voltage of 15 KV after sputter coating the samples with Platinum (JEOL JSC-1200 fine coater, Japan). SEM images for different electrospun gelatin/collagen fibers samples were then analyzed using Image Analysis Software (Image J, National Institute of Health, USA) to calculate the average fiber diameter. The diameter of approximately 100 randomly selected nanofibers was measured to obtain the average nanofiber diameter in each gelatin/collagen fibers sample.

### Contact Angle Measurement

The hydrophilic/hydrophobic properties of electrospun fiber mats were measured by sessile drop water contact angle measurement using a VCA Optima Surface Analysis system (AST products, Billerica, Mass.). Distilled water was used for drop formation.

### Release Profile of Polyhydroxy Antimicrobial Agent

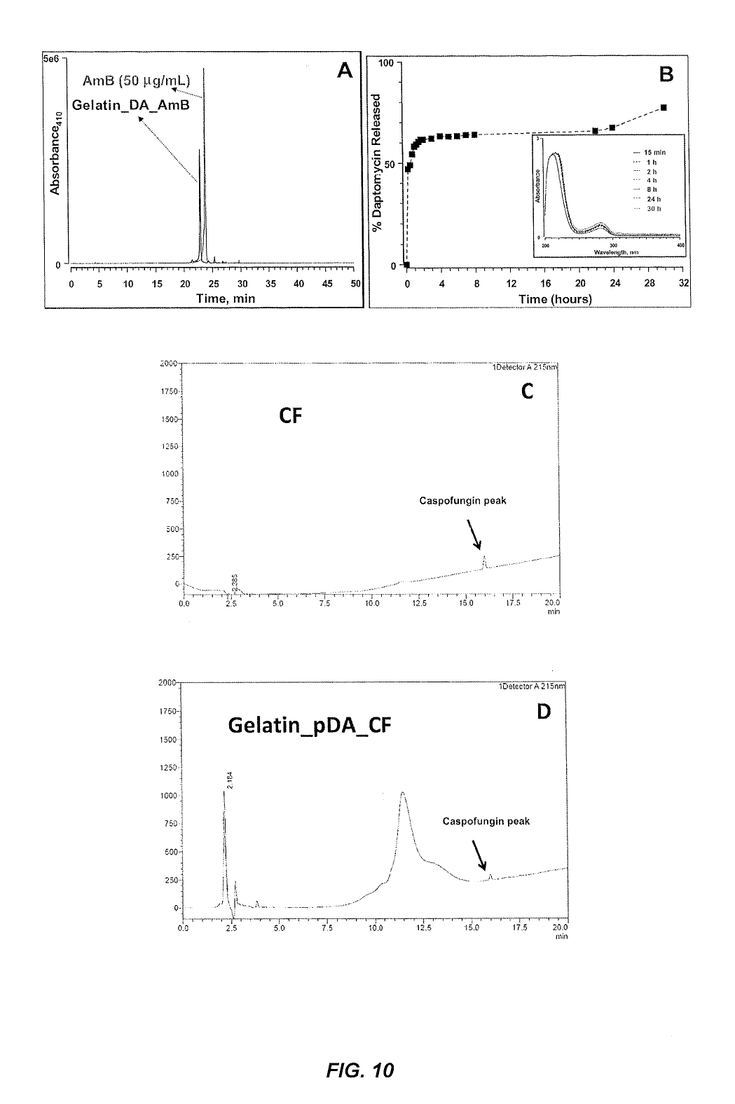

The amount of antimicrobial agent released from electrospun dopamine coated gelatin fiber mats with polyhydroxy antimicrobial agent were determined as follows. 10 mg of each respective fiber mat (containing 50 μg of the antimicrobial agent based on calculation) was immersed in 1 mL of PBS buffer (pH 7.5) in a 24-well plates (Nunc®). At pre-determined intervals, the solution was monitored by the UV absorbance (280 nm) using a UV-spectrophotometer (UV1800 double beam spectrophotometer, Shimadzu, Kyoto, Japan) and the amount of antimicrobial agent released (FIG. 10B) was estimated by calibration method using known standard solutions.

The aforementioned method was applicable for daptomycin only. For amphotericin B- and caspofungin-loaded electrospun fiber mats, the amount of antimicrobial agent released could not be determined using the aforementioned method because these drugs were most likely covalently linked to the fiber mats. For instance, to determine the amount of bound amphotericin B, 10 mg of electrospun polydopamine coated gelatin fiber mats containing 50 μg (calculated value) of the amphotericin B was sonicated in 1 mL of water for 30 min to release amphotericin B from the fiber mat. The amount of amphotericin B released (FIG. 10A) into the supernatant after sonication of the electrospun polydopamine fiber mat containing amphotericin B were determined by reverse phase-high performance liquid chromatography (RP-HPLC). For caspofungin, 5 mg of electrospun polydopamine coated fiber mats containing 25 μg (calculated value) of caspofungin was sonicated in 1 mL of water for 30 min to release amphotericin B from the fiber mat. The amount of caspofungin released (FIG. 10D) into the supernatant after sonication of the electrospun polydopamine fiber mat containing caspofungin was determined by RP-HPLC by comparing with the RP-HPLC profile of 25 μg caspofungin (FIG. 10C).

RP-HPLC was carried out using Waters HPLC analyzer with C18 analytical column (Phenomenex, Calif., USA). The programming of the mobile phases (A=distilled water with 1% trifluoroacetic acid, B=acetonitrile with 1 trifluoroacetic acid) were as follows: a static mode of 95:5 (A:B, v:v) from 0-5 min, a gradient mode of 10:90 from 5-35 min, a gradient mode of 5:95 from 35-40 min, a gradient mode of 95:5 from 40-43 min and static mode of 95:5 from 43-50 min. A UV-Visible detector was used and the detector wavelength was 280 nm (100 μl sample injection).

The difference in area under the RP-HPLC curves of 50 μg/mL amphotericin B (AmB) and for amphotericin B released on sonication from electrospun dopamine coated gelatin fiber mats with amphotericin B (Gelatin_DA-AmB) provides an estimate of the amount of amphotericin B released from the fiber mats on sonication (see FIG. 10A).

### Radial Disc Diffusion Assay

Fungus or yeast cell cultures (at a concentration of 0.5 McFarland standards) were spread onto the surface of sterile Sabouraud dextrose agar plates using a cotton swab in 9 cm diameter petri dishes. Samples of each fiber mat (1 cm×1 cm) were placed on top of the swabbed cultures and incubated at 37° C. Antifungal activity of the fiber mats was visualized as the diameter of the zone of inhibition after incubating plates in the dark for 48 hours for Candida strains and 72 hours for Fusarium and Aspergillus strains. The assay was performed in two independent duplicates and the average value was reported.

Gram positive and Gram negative bacterial cultures (at a concentration of 0.5 McFarland standards) were spread onto the surface of sterile Muller and Hinton agar (MHA) plates using a cotton swab in 9 cm diameter Petri dishes. Each electrospun dopamine-coated gelatin antimicrobial fiber mats to be tested (1 cm×1 cm) was placed on top of the swabbed cultures in separate Petri dishes and incubated at 37° C. Anti-bacterial activity of these electrospun mats were observed as the zone of inhibition (in cm) after incubating the Petri dishes in the dark for 24 h. The assay was performed in two independent duplicates and the average value was reported.

### Long-Term Antimicrobial Efficacy Assay

The long-term antimicrobial efficacy of the electrospun dopamine coated gelatin fiber mats with antimicrobial agent (not exposed to ammonium carbonate) and electrospun polydopamine cross-linked gelatin fiber mats with antimicrobial agent after exposure to ammonium carbonate were assessed by a modified ASTM protocol (ASTM). Briefly, each antimicrobial fiber mat was immersed in phosphate buffered saline (PBS) pH 7.0 with constant shaking. At predetermined intervals, the mat was removed, rinsed with water and assay for antimicrobial activity by the disc diffusion method as described above. For assessing antifungal activity, C. albicans ATCC 10231 was used. For assessing the antibacterial activity, MRSA 9808R was used.

### Aqueous Stability

Electrospun fiber mats were soaked in PBS over various defined time periods and the morphology of each fiber mats after soaking were observed by SEM as described above.

### Example 2: Results

### Properties of Electrospun Fiber Mats

Scanning Electron Microscopy.

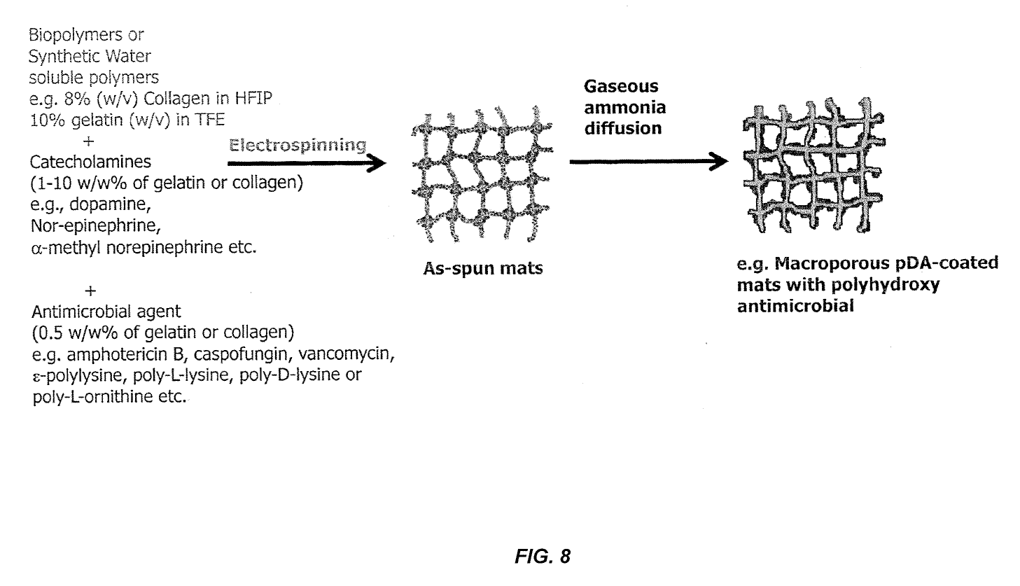

Electrospun polydopamine cross-linked gelatin fiber mats with various antimicrobial agents after exposure to ammonium carbonate displayed tight fusion of the electrospun fibers (FIG. 8), indicating stabilization of the fibers at the junctions.

Contact Angle Measurements.

Water contact angles (θ_{static}) of the electrospun polydopamine cross-linked gelatin fiber mats after exposure to ammonium carbonate with various antimicrobial agents were measured to determine the wettability of the mats. The results suggest that electrospun dopamine coated antimicrobial fiber mats increase the hydrophobicity of the fiber surface compared to electrospun polydopamine coated fiber mats with no antimicrobial agent (Table 1). Electrospun polydopamine cross-linked fiber mats with amphotericin B after ammonium carbonate exposure displayed the lowest contact angles compared to the other antimicrobial agents.

TABLE 1
Water contact angle measurement of electrospun
polydopamine cross-linked gelatin fiber mats containing
various antimicrobial agents after exposure to ammonium carbonate

| Antimicrobial agent | Contact Angle, ° |
|---|---|
| Electrospun polydopamine coated fiber | 64.7 ± 1.2 |
| mat with no antimicrobial agent | |
| Amphotericin B | 26.2 ± 0.6 |
| Caspofungin | 99.8 ± 1.0 |
| Daptomycin | 92.2 ± 2.2 |
| Vancomycin | 99.6 ± 0.5 |
| Polymyxin B | 98.8 ± 2.2 |

Release Profile Assessed by RP-HPLC.

The amounts of amphotericin B or caspofungin released from the electrospun polydopamine cross-linked gelatin fiber mats with the applicable antifungal after exposure to ammonium carbonate was very low and could not be quantified. These results suggest that these two polyhydroxy antifungals could be covalently linked to the fiber mat. The amount of amphotericin B released from the supernatant after sonication of electrospun polydopamine cross-linked gelatin fiber mats with amphotericin B by reverse phase-high performance liquid chromatography (RP-HPLC). Chromatographic analysis of the supernatant indicated that approximately 49.5±3% of amphotericin B was released upon sonication (FIG. 10A) implying that ˜50% of amphotericin B remained bound with the fiber mat.

In the case of caspofungin, even less released caspofungin (about 35%) was detected in the supernatant suggesting that about 65% of caspofungin remained bound to the fiber mat after sonication (FIGS. 10C and 10D).

In the case of daptomycin, the release studies indicated that >60% of daptomycin was released within 5 h and ˜80% of the daptomycin was released within 30 h (FIG. 10B). Daptomycin which has less hydroxyl groups compared to caspofungin and amphotericin B was observed to be released in greater amounts. These results suggest the possible role of multiple hydroxyl groups in stabilizing the drug-matrix interactions.

Antimicrobial Activity—Radial Diffusion Assay.

Radial diffusion assays were performed against a panel of pathogenic Gram-positive bacteria and yeasts/fungi. The electrospun polydopamine cross-linked gelatin fiber mats with amphotericin B or caspofungin after exposure to ammonium carbonate retained their antifungal properties against pathogenic yeasts and fungi (Table 2). The reported values are an average of two-independent measurements.

TABLE 2
Antimicrobial properties of electrospun polydopamine
cross-linked gelatin fiber mats with amphotericin B or
caspofungin after exposure to ammonium carbonate
measured by radial diffusion assay.

Zone of inhibition (in
cm) of fiber mat with

| Fungi strain | Amphotericin B | Caspofungin |
|---|---|---|
| C. albicans 10231 | 2.8 | 2.8 |
| C. albicans 2091 | 2.4 | 2.7 |
| C. albicans 24433 | 2.1 | 2.3 |
| C. albicans 1976R | 2.1 | 2.7 |
| C. albicans 2672R | 2.2 | 2.8 |
| A. fumigatus 16404 | 2.0 | 2.5 |
| A. braslillineus 90906 | 1.9 | 3.5 |

Similarly, a clear zone of inhibition was observed for electrospun polydopamine cross-linked gelatin fiber mats with vancomycin or daptomycin after exposure to ammonium carbonate against Gram-positive bacteria strains (Table 3).

TABLE 3
Antimicrobial properties of electrospun polydopamine
cross-linked gelatin fiber mats with vancomycin or daptomycin
after exposure to ammonium carbonate measured by
radial diffusion assay.

Zone of inhibition
(in cm) of fiber mat with

| Bacteria strain | Vancomycin | Daptomycin |
|---|---|---|
| S. aureus DM 4001R | 2.8 ± 0.2 | 1.95 ± 0.05 |
| S. aureus DM4583R | 2.75 ± 0.15 | ND |
| S. aureus DM4400R | 2.3 ± 0.2 | 2.1 ± 0.1 |
| MRSA 21595 | 2.8 ± 0.2 | ND |
| MRSA 21455 | 3.1 ± 0.1 | 1.8 ± 0.1 |
| MRSA 9808R | 2.5 ± 0.1 | 2.05 ± 0.5 |
| E. faecalis 29212 | 2.95 ± 0.15 | 1.4 ± 0.1 |
| E. hirae 9790 | 3.1* | 1.35 ± 0.15 |

MRSA—methiciilin resistant S., aureus.
ND—not determined
*Standard deviation not observed from repeats.

The results in Tables 2 and 3 suggest that the polydopamine crosslinks did not affect the antimicrobial properties of the antimicrobial agents in the fiber mats. In addition, no colonies were detected on top as well as at the bottom of the mats, indicating complete inhibition of microbes.

Long Term Antimicrobial Efficacy.

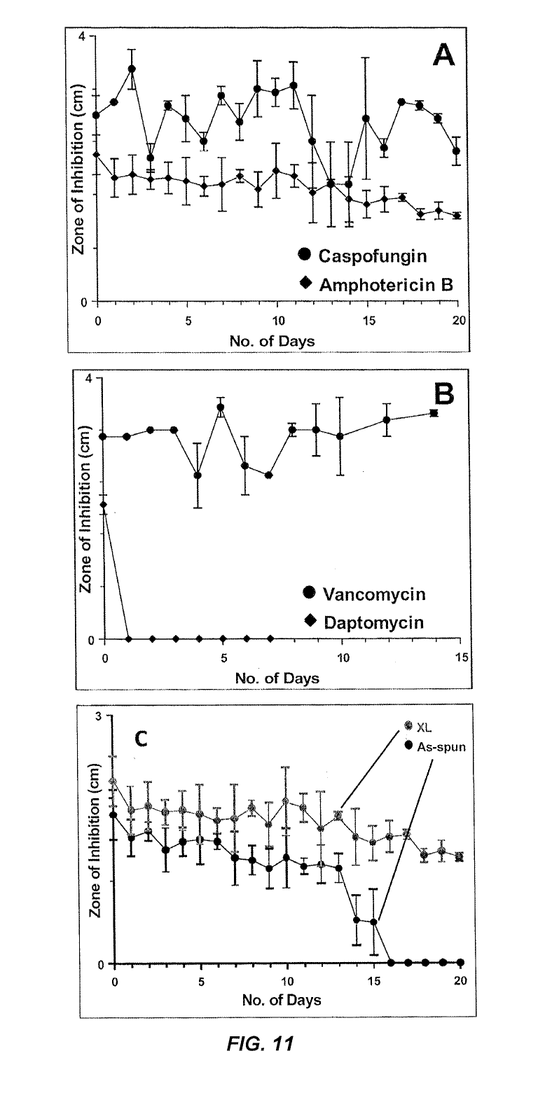

The long term antimicrobial efficacy of the various fiber mats were assessed by a modified ASTM protocol as described above. Electrospun polydopamine cross-linked gelatin fiber mats with amphotericin B after exposure to ammonium carbonate retained antifungal activity for an extended period of at least 20 days (FIG. 11A). Similarly, electrospun polydopaminecoated gelatin fiber mats containing caspofungin after exposure to ammonium carbonate retained antifungal activity for a period of 20 days as well (FIG. 11A). The long-term antimicrobial activity for electrospun polydopamine cross-linked gelatin fiber mats with vancomycin after exposure to ammonium carbonate was observed to last for at least 15 days (FIG. 11B). However, electrospun polydopamine cross-linked gelatin fiber mats with daptomycin did not show a clear zone of inhibition from after the first day. These results are consistent with the earlier observations that rapid release of daptomycin from the fiber mat occurred within 24 h, as this would have resulted in a low amount of daptomycin remaining in the fiber mat and hence the loss of antibacterial activity.

Further, the antimicrobial activity for electrospun polydopamine cross-linked gelatin fiber mats with amphotericin B before (As-spun) exposure to ammonium carbonate was observed to last for only 15 days while those cross-linked after exposure to ammonium carbonate (XL) showed antimicrobial activity up to 20 days and possibly beyond (FIG. 11C). A larger zone of inhibition was also observed for electrospun polydopamine cross-linked gelatin fiber mats with amphotericin B after exposure to ammonium carbonate when compared to electrospun polydopamine cross-linked gelatin fiber mats with amphotericin B before exposure to ammonium carbonate. This result suggests that exposure to ammonia carbonate improves clinical and long-term efficacy of fiber mats in respect to antimicrobial delivery.

In this study, it was observed that the antimicrobial activity of electrospun polydopamine cross-linked gelatin mats with polyhydroxy antifungal agents (amphotericin B and caspofungin) and vancomycin (an antibacterial agent) persisted for at least 15 days and this could be due to the strong interaction between the polyhydroxy antifungals and the fiber coupled with the “glue-like” properties of polydopamine.

Aqueous Stability.

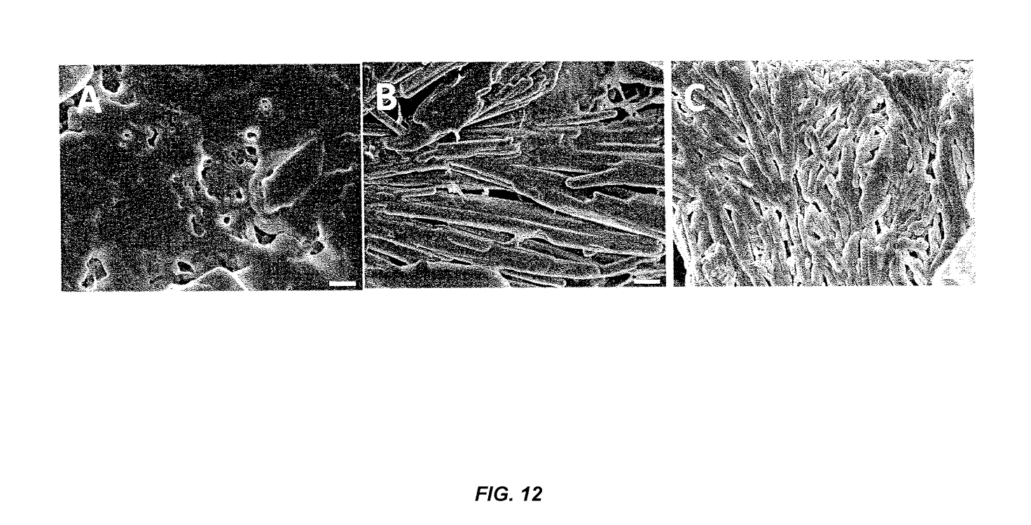

The aqueous stability of electrospun pristine gelatin fiber mats, electrospun polydopamine cross-linked gelatin fiber mats after exposure to ammonium carbonate and electrospun polydopamine cross-linked gelatin fiber mats with an antifungal agent after exposure to ammonium carbonate was investigated by soaking them in PBS and observing the morphological changes. Electrospun pristine gelatin fiber mats readily formed a clear and transparent gel and loss of fibrous structure was observed within 5 h after immersion in PBS (FIG. 12A). The electrospun polydopamine cross-linked gelatin fiber mats after exposure to ammonium carbonate soaked in PBS for 1 week displayed whisker-like structures (FIG. 12B). As the incubation time progressed to 2 weeks, the average diameters of the whisker-like structures decreased and coalesce to form film-like structures with irregular morphologies (FIG. 12C).

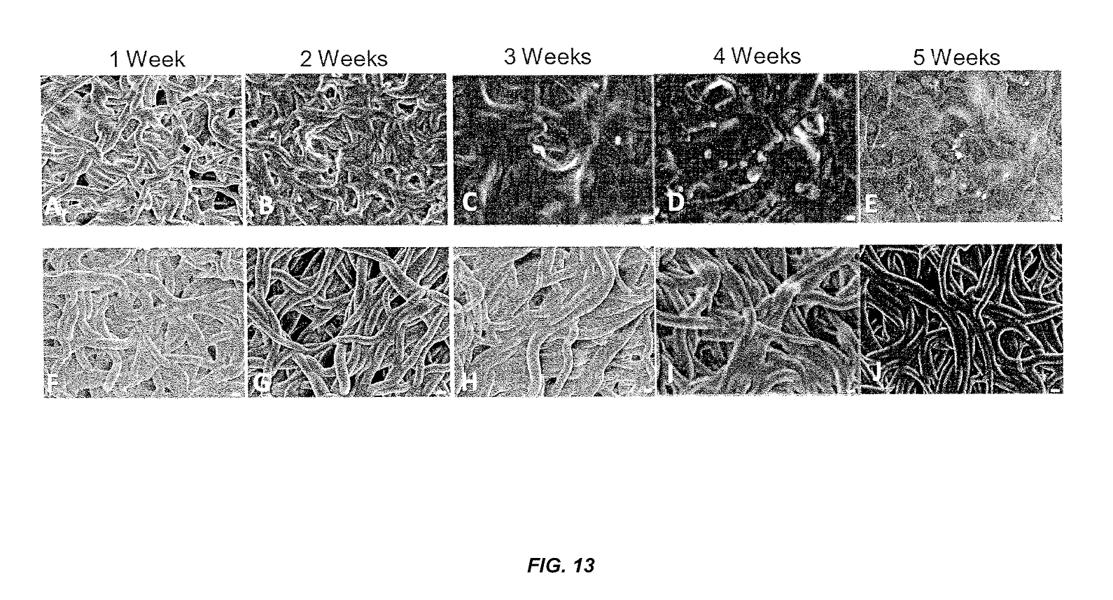

For electrospun polydopamine cross-linked gelatin fiber mats with amphotericin B or caspofungin after ammonium carbonate exposure, the individual fibers remained intact though appeared curly after 1 week in PBS (FIG. 13A and FIG. 13F). After 2 weeks of incubation, considerable fusion of the fibers was noticeable in the fiber mats with amphotericin B (FIG. 13B), and fibrous morphology disappeared completely as the incubation time reached 3 weeks and beyond (FIGS. 13C-13E).

However, the fiber mats with caspofungin remained intact, although a decrease in the average fiber diameter and coalescence of individual fibers were noticeable when the incubation time reached 4 and 5 weeks. It should be noted that both amphotericin B and caspofungin contain significant number of free —OH and —NH₂ groups which may enhance the cross-linking density during polydopamine coating.

The results suggests that electrospun polydopamine cross-linked gelatin fiber mats with an antifungal agent after exposure to ammonium carbonate have a higher aqueous stability when compared to electrospun polydopamine cross-linked gelatin fiber mats without an antifungal agent (FIG. 13A and FIG. 13F vs. FIG. 12B).

### Discussion

As described above, electrospun fibers can be cross-linked by catecholamines or polyphenolic compounds. The present invention extends further by including polyhydroxy antimicrobial agents. The results suggest that electrospun gelatin mats with catecholamine and polyhydroxy antimicrobial agents after exposure to ammonium carbonate are durable and have significant biological properties. The fiber mats with antimicrobial agents inhibited the growth of pathogenic microorganisms and retained the structure of the nanofibers for an extended period of time.

### Example 4. Electrospinning of Collagen and Composite Nanofibers and Morphological Characterization

Reagents:

Atelocollagen powder (Collagen Type I, Product No. CLP-01) from bovine dermis was a product of Koken (Tokyo, Japan) and purchased from Unison Collaborative Pte Ltd. Dopamine hydrochloride (DA), norepinephrine hydrochloride (NE), 1,1,1,3,3,3-hexafluoro-2-propanol (HFIP), calcium chloride (CaCl₂), and ammonium carbonate were obtained from Sigma-Aldrich (Singapore). Chemicals were of analytical grade and were used without further purification.

Electrospinning of Collagen and Composite Nanofibers.

Pristine collagen mats were prepared from 8% w/v dope solution in hexafluoro isopropanol (HFIP) and the solution was transferred to a polypropylene plastic syringe with 27 G stainless steel blunted needle. The solution was extruded at an applied voltage of 13 kV from a high voltage power supply (Gamma High Voltage Research, Inc., FL, USA) and the distance between needle and collector (a flattened aluminum foil) was set at 17 cm at a feed rate of 1 ml/h (KD 100 Scientific Inc., MA, USA). Identical conditions were used for the preparation of mats containing 10% (w/w) catecholamines. However, for the preparation of mats containing catecholamines and CaCl₂, collagen was dissolved in 90% HFIP and 10% H₂O containing CaCl₂ (final concentration of 20 mM Ca²⁺). The voltage/distance of 17 kV/13 cm and a feed rate of 0.8 mL/h produced bead-free nanofibers. All the electrospinning experiments were performed at room temperature at around 25% humidity and the collected electrospun mats were vacuum dried for 48 h to remove any residual HFIP and then stored in dry cabinets for ammonium carbonate exposure and characterization. The catecholamine-loaded mats with or without Ca²⁺ were placed in a sealed desiccator containing ˜5 g of (NH₄)₂CO₃ powder for 24 h to induce oxidative polymerization and precipitation of CaCO₃. For SEM, photoluminescence and cell culture studies, the fibers were collected on microscopy cover slips (15 mm) and on gold-coated copper grids for TEM. For simplicity, the mats are labelled as follows: pristine collagen mats—ES_Coll; As-spun collagen mats with DA or NE—Coll_DA or Coll_NE; Collagen mats after (NH₄)₂CO₃ exposure—Coll_pDA and Coll_pNE; As-spun collagen mats containing DA or NE and 20 mM Ca²⁺—Coll_DA_Ca or Coll_NE_Ca; Collagen mats containing DA or NE and 20 mM Ca²⁺ after (NH₄)₂CO₃ exposure—Coll_pDA_Ca or Coll_pNE_Ca.

Morphological Characterization by Scanning and Transmission Electron Microscopies (SEM/TEM).

Morphological analysis of collagen mats were investigated by field emission scanning electron microscopy (FE-SEM) to infer i) the influence of incorporating catecholamines within collagen scaffold, ii) the effect of integrating Ca²⁺ ions in the catecholamine-loaded mats, iii) the influence of crosslinking treatment on the morphology of the electrospun collagen nanofibers, and iv) to study the fracture morphology of the various scaffolds after uniaxial tensile testing. SEM studies were also performed to check the cell adhesion and cellular morphology seeded onto the various scaffolds. SEM analysis was performed on a FE-SEM (FEI-QUANTA 200F, the Netherlands) at an accelerating voltage of 15 KV after sputter coating the samples with Platinum (JEOL JSC-1200 fine coater, Japan). Image Analysis Software (Image J, National Institute of Health, USA) software was used for estimating the average fiber diameter for various scaffolds. For calculating the average diameter of each sample, ˜100 nanofibers from their respective SEM images (3-4 micrographs focusing different areas) were randomly selected and used for measuring their diameter. Selected area electron diffraction (SAED) patterns and TEM studies were performed using JEOL JEM-3010 instrument.

Results.

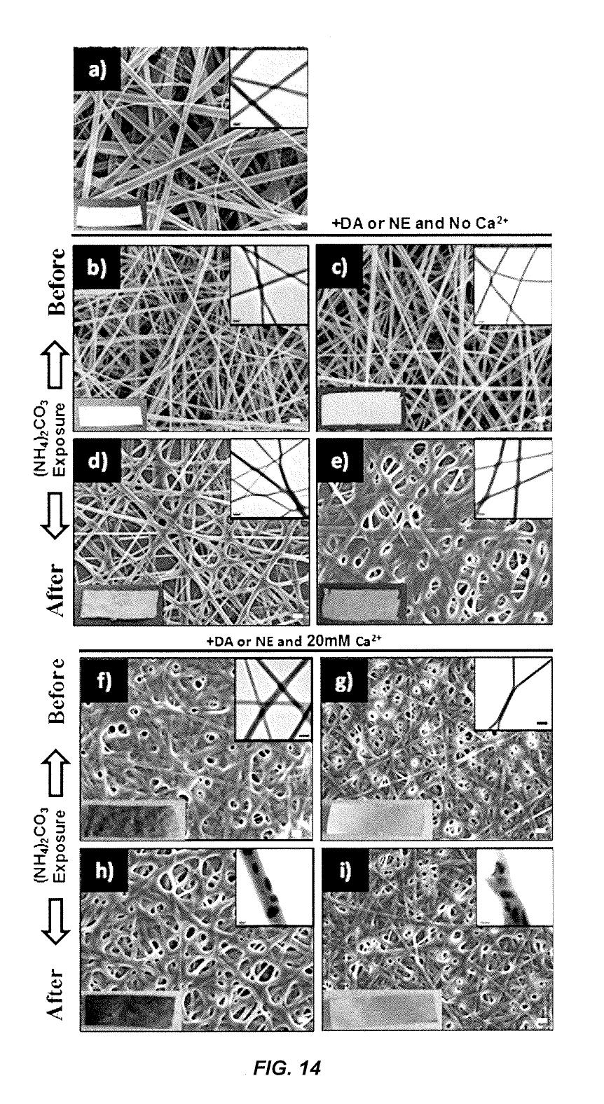

Morphological features of ES collagen scaffolds with or without catecholamines and catecholamines/Ca²⁺ were investigated by electron microscopy. Scanning Electron Microscopy (SEM) of pristine collagen mats electrospun from a 10% dope solution in HFIP revealed the presence of smooth, bead-free and uniform fiber morphology with an average diameter (Ø) of 900±150 nm (FIG. 14A).

Addition of 10% (w/w) catecholamines caused two noteworthy morphological changes in the electrospun collagen mats: i) a significant decrease (˜3 folds) in the Ø-values (331±46 nm for COLL_DA and 323±43 nm COLL_NE) and ii) presence of catecholamines merge the two fibers and remove the identity of individual fibers at the contact points, thus forming welded or soldered junctions (FIGS. 14B, 14C and insets at top right). Since the electrospinning was performed in a poorly hydrogen bonded solvent which promotes intramolecular hydrogen bonding, the presence of higher concentration of catecholamines at the contact points might promote the inter fiber adhesion, thus forming welded junctions. See, Vidal 2014; He 2011.

Previous studies suggested that electrospun collagen mats prepared from fluoro alcohols lack adequate mechanical strength and aqueous stability, therefore may not be suitable for oxidative polymerization of catecholamines by conventional alkaline Tris-HCl (pH 8.5) method. See, Zeugolis 2008; Matthews 2002. To avoid the deleterious effect of alkaline solution on collagen nanofibers, we exposed the catecholamine-loaded mats to (NH₄)₂CO₃ in order to induce the oxidative polymerization of catecholamines. Henceforth, this method is referred as ammonium carbonate diffusion method (ADM). Since the entire reaction was carried out at solid-vapor interface, one would expect the morphological features of electrospun collagen remain intact. Indeed, substantial increase in the formation of welded junctions was observed after exposure of the catecholamines-loaded mats to (NH₄)₂CO₃ (FIGS. 14D, 14E). The effect was remarkable in the case of NE containing mats, as numerous inter fiber adhesion and welded junctions resulted in discontinuous porous coatings after ADM (FIG. 14E). Brown coloration of the scaffolds after ADM further confirmed the formation of oxidative products of catecholamines (Insets at bottom left of FIGS. 14E, 14D).

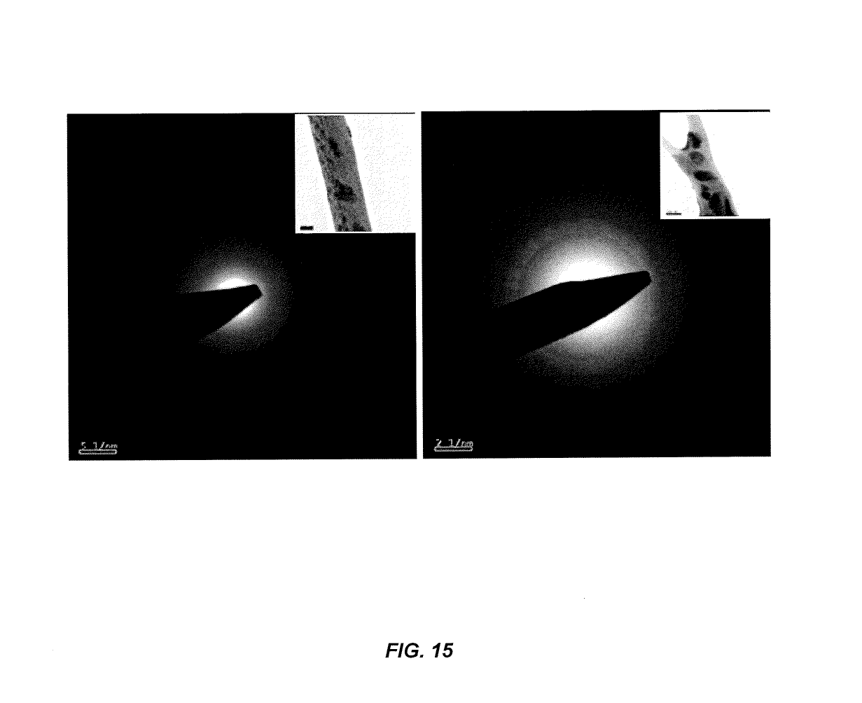

Notably, addition of 20 mM Ca²⁺ to collagen-catecholamine dope solution dramatically increased the formation of welded junctions in comparison to mats electrospun without Ca²⁺ (FIGS. 14F, 14G and insets at top right). The color of the mats changed from white to brown (DA)/pale pink (NE) upon addition of 20 mM Ca²⁺ (FIGS. 14F, 14G and insets at bottom left). These results suggest possible cation-induced electrochemical oxidation of catecholamines to polycatecholamines that possess inherent adhesive properties, thus promoting inter fiber adhesion. See, Chai 2014. As the color of the dope solution containing catecholamines and Ca²⁺ did not change prior to electrospinning, it is likely that the brown coloration of the mats could be attributed to the electrochemical potential-induced oxidation of DA and NE during the electrospinning process. See, Robinson 2003; Li 2010. After ADM, the catecholamines-Ca²⁺ loaded collagen mats displayed more intense coloration without profound changes in the morphology (FIGS. 14H, 14I). High-resolution TEM images showed distribution of sub-nm sized nanoparticles along the length of individual fibers, indicating the formation of CaCO₃ (inset at top right of FIGS. 14H, 14I). The appearance of diffused rings in the selected area diffraction pattern confirmed the amorphous nature of the particles (FIG. 15). Detailed mineralogical characterization of the particles was difficult as we could not detect clear signals by FTIR, Raman or XRD methods. Nevertheless, taking advantage of the formation of gaseous ammonia and CO₂ upon decomposition of (NH₄)₂CO₃, we reported a simple strategy to carry out simultaneous crosslinking and mineralization of ES collagen fibers.

### Example 5. Wettability Study of Composite Nanofiber Polymer Product

Contact Angle Measurements:

To evaluate the hydrophilic/hydrophobic characteristics of the different electrospun collagen fiber mats sessile drop static and dynamic water contact angle measurements using a VCA Optima Surface Analysis system (AST products, Billerica, Mass.) were performed. A 1 μL drop of distilled water was placed on the fiber mats and photographed continuously at every 10 sec for 60 seconds. The linear part of the non-linear decrease in contact angle—time curve was extrapolated to 0 time to determine θ_{static} values. The reported values were determined from two independent triplicate experiments.

Wettability of the Electrospun Collagen Mats:

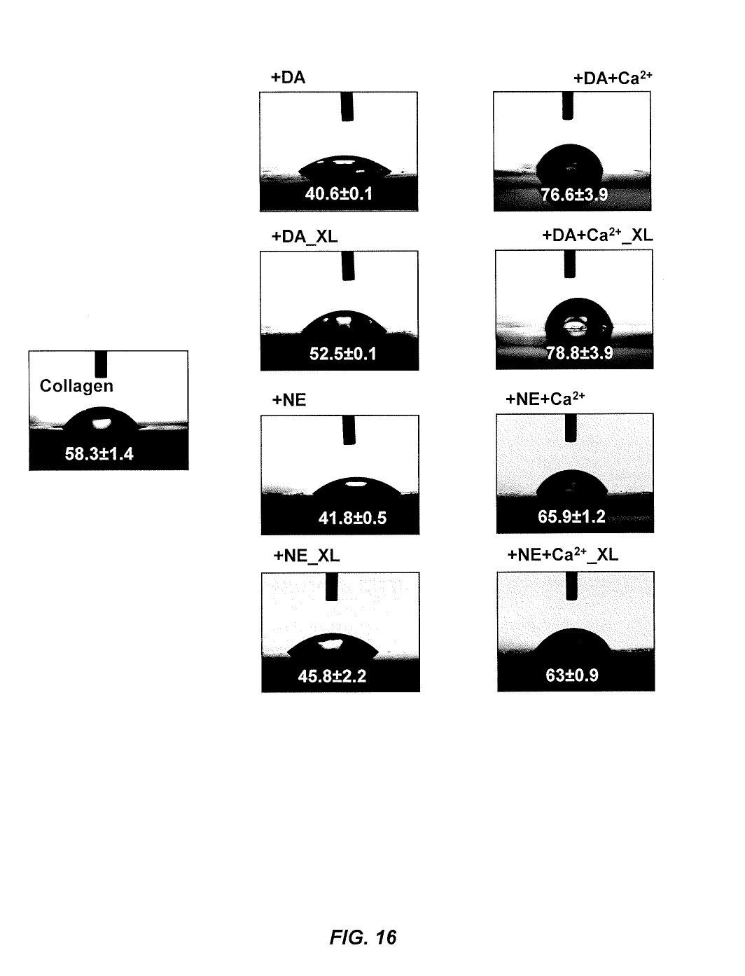

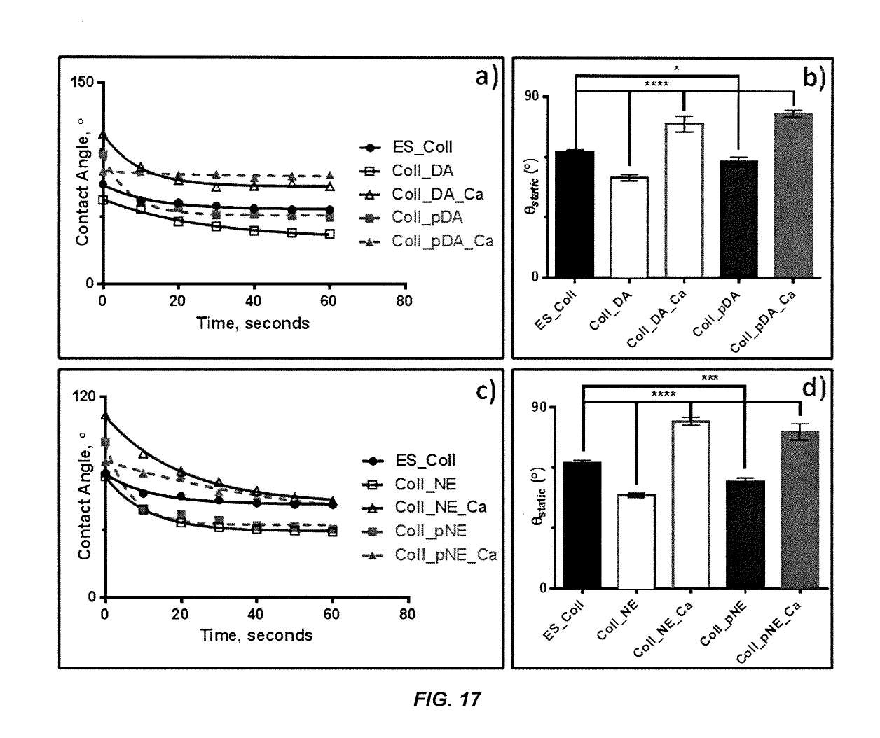

The wettability of the mats was assessed by dynamic water contact angle measurements and θ_{static} values were reported. FIG. 16 shows the changes in contact angles of electrospun collagen prepared under various conditions. For pristine collagen mats (ES_Coll), the water contact angle (WCA) reached up to 59.3±1.4° in 60 seconds from an initial value of 74.4±0.1° (FIG. 17A).

Incorporation of DA (Coll_DA), decreased the initial WCA by about 20°, indicating increased wettability of the mats and reached a final value of 40.6±0.1° after 1 min. After ADM treatment (Coll_pDA), the initial WCA decreased sharply from 92.1±4.5° to a final value of 52.5±0.1°. The presence of Ca²⁺ in the dope solution (Coll_DA_Ca) has dramatic effect on the initial WCA and the values decreased exponentially and reached a value of 76.7±3.9°. Interestingly, after ADM exposure, no apparent change in the WCA was observed for the Coll_pDA_Ca.

To shed further insight into the WCA measurements, we determined the static WCA (θ_{static}), by extrapolation of the linear part of the curve to zero time. The results indicated that collagen mats containing DA before or after ADM decreased θ_{static} when compared to pristine collagen mats (FIG. 17B). In contrast, the values increased significantly for Coll_DA_Ca mats and remained unaltered after ADM (FIG. 17B). These results corroborate our earlier observations that the presence of Ca²⁺ together with DA could trigger the oxidative polymerization during electrospinning, thus producing mats with surface wettability that was similar to Coll_pDA mats. In the case of ES collagen mats containing NE, a similar trend in the time-dependent WCA (FIG. 17C) and θ_{static} were observed (FIG. 17D).

### Example 6. XPS Analysis of Composite Nanofiber Polymer Product

X-Ray Photoelectron Spectroscopy.

XPS studies were carried out using Kratos AXIS UltraDLD (Kratos Analytical Ltd) in ultrahigh vacuum (UHV) conditions of ˜10⁻⁹ Torr by employing a monochromatic Al—Kα X-ray source (1486.71 eV). The general scan and different high resolution spectra were recorded for in-depth analysis of various chemical states of fabricated samples. During analysis, the high resolution spectra were deconvoluted using various Gaussian-Lorentzian components with the background subtracted in Shirley mode.

Characterization of Collagen Composites by XPS.

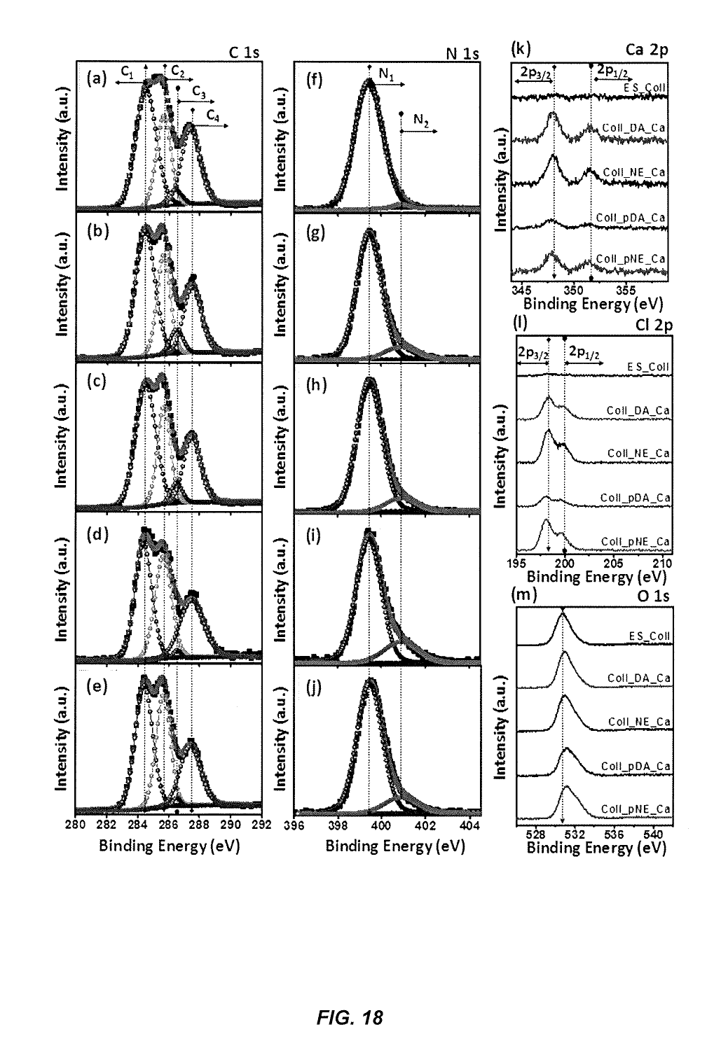

We next focused our attention on the characterization of composite structures formed. To infer the changes in chemical bonding environments around carbon and nitrogen of collagen fibers upon incorporation of catecholamines (DA and NE) with CaCl₂ followed by ADM, we performed the XPS measurements. The shape of high resolution C 1s and N 1s core-level spectra indicated the presence of multiple bonding components in the electrospun mats (FIG. 18). Deconvolution of C 1s spectra revealed the presence of four peaks in each sample; namely C₁, C₂, C₃ and C₄ which correspond to C—C/C—H, C—N, C—O and C═O bonding, respectively (FIGS. 18A-18J). Similarly, the deconvolution of N 1s spectra revealed the presence of two peaks in each sample; namely N₁ and N₂ which are assigned to R₂NH and RNH₂ bonding, respectively. The peak positions for these bonding are provided in Table 4 and are found to be in good agreement with the reported literature. See, Zangmeister 2013; He 2014.

TABLE 4
Peak positions which correspond to
various bonding states of C1s and N1s peaks.

| Peak | Peak Position (eV) | Bond Type |
|---|---|---|
| C₁ | 284.5 ± 0.1 | C—C/C—H |
| C₂ | 285.65 ± 0.05 | C—N |
| C₃ | 286.5 ± 0.1 | C—O |
| C₄ | 287.5 ± 0.1 | C═O |
| N₁ | 399.42 | R₂NH |
| N₂ | 400.9 | RNH₂ |

Furthermore, an area ratio method was used to estimate the bonding content of each peak for all samples and the results are summarized in Table 5. C 1s and N1s spectra of Coll_DA_Ca and Coll_NE_Ca samples revealed significant increase in C—N bonding intensity and atomic composition, corroborating the color changes observed during electrospinning. The increase in C—N bonding intensity was more pronounced after ADM, thus confirming the conversion of catecholamines to polycatecholamines.

TABLE 5
Quantitative analysis of various bonds
examined from C1s core level spectra.

| | Percentage of the Constituent Peaks | | | |
|---|---|---|---|---|
| | C1s Core Level | | | |
| | C—C/C—H | | | |
| Samples | (%) | C—N (%) | C—O (%) | C═O (%) |
| ES_Coll | 45.6 | 24.5 | 3.9 | 26 |
| Coll_DA_Ca | 46.7 | 25 | 6 | 22.3 |
| Coll_NE_Ca | 48.3 | 25 | 4.7 | 22 |
| Coll_pDA_Ca | 38.8 | 33.5 | 1.1 | 26.6 |
| Coll_pNE_Ca | 42.6 | 32 | 1.3 | 24.1 |

To probe the modifications of bonding environments around calcium ions, Ca 2p and Cl 2p core level spectra were also recorded for all samples (FIGS. 18K, 18L). For Coll_DA_Ca and Coll_NE_Ca samples, the Ca 2p_{3/2} peak at 348.0 eV and Cl 2p_{3/2} (at 198.3 eV) and Cl 2p_{1/2} (at 199.85 eV) peaks indicate that the bonding environments of calcium or chloride ions did not change after electrospinning. See, Hu 2006; Demri 1995; Baltrusaitis 2007. However, two important observations confirmed the formation of CaCO₃ in the mats after ADM. First, the intensity of Ca and Cl peaks decreased in both Coll_pDA_Ca and Coll_pNE_Ca mats, though the effect was substantial in the mats containing dopamine. Second, the Ca 2p_{3/2} (347.6±0.05 eV) and the Cl 2p_{3/2} (at 198.0 eV) peaks shifted towards lower binding energy, when compared to Coll_DA_Ca or Coll_NE_Ca mats. The appearance of Ca 2p_{3/2} in the range 346.5-347.9 eV have been reported for various polymorphs of CaCO₃. See, Blanchar 1992; Kouhi 2013; Chu 2013.

To obtain further insight into the formation of CaCO₃ in the mats after ADM, O 1s core level spectra were examined for the cross-linked nanofibers. A weak up shift in the position of O 1s peak from 530.8 eV to 531.0 eV was observed ES_Coll upon incorporation of DA/NE and Ca²⁺ i.e., for Coll_DA/NE_Ca mats (FIG. 18M). Since Ca 2p and Cl 2p spectra revealed no apparent change in the spectra for Coll_DA/NE_Ca mats, the marginal up shifting of O 1s peak in these samples could be due to weak interactions of Ca²⁺ with catecholamines. However, after ADM, the O 1s peak was further up shifted to 531.2 eV for Coll_pDA/pNE_Ca mats, confirming the formation of carbonate structures in these mats. See, Demri 1995; Baltrusaitis 2007; Blanchard 1992. Together with TEM results, these observations confirmed the formation of CaCO₃ in the mats after (NH4)₂CO₃ treatment.

### Example 7. Mechanical Characterization of Composite Nanofiber Polymer Product

Determination of Mechanical Properties.

A tabletop tensile tester (Instron 5345, USA) using a load cell of 10 N capacities at ambient conditions was used to carry out the tensile testing of the electrospun fibers in accordance with the ASTMD882-02 protocol. All the mechanical properties (Tensile strength, Failure Strain, Young's Modulus and Work of Failure) were calculated based on the generated stress-strain curves for each collagen mat. The mats were cut into rectangular strips of 1 cm×3 cm and the thickness of each sample was measured using a micrometre calliper. Finally, the samples were mounted vertically on the gripping unit of the tensile tester at a cross-head speed of 5 mm min⁻¹. The average results are reported from 2-4 independent measurements.

Mechanical Properties of Collagen Composites.

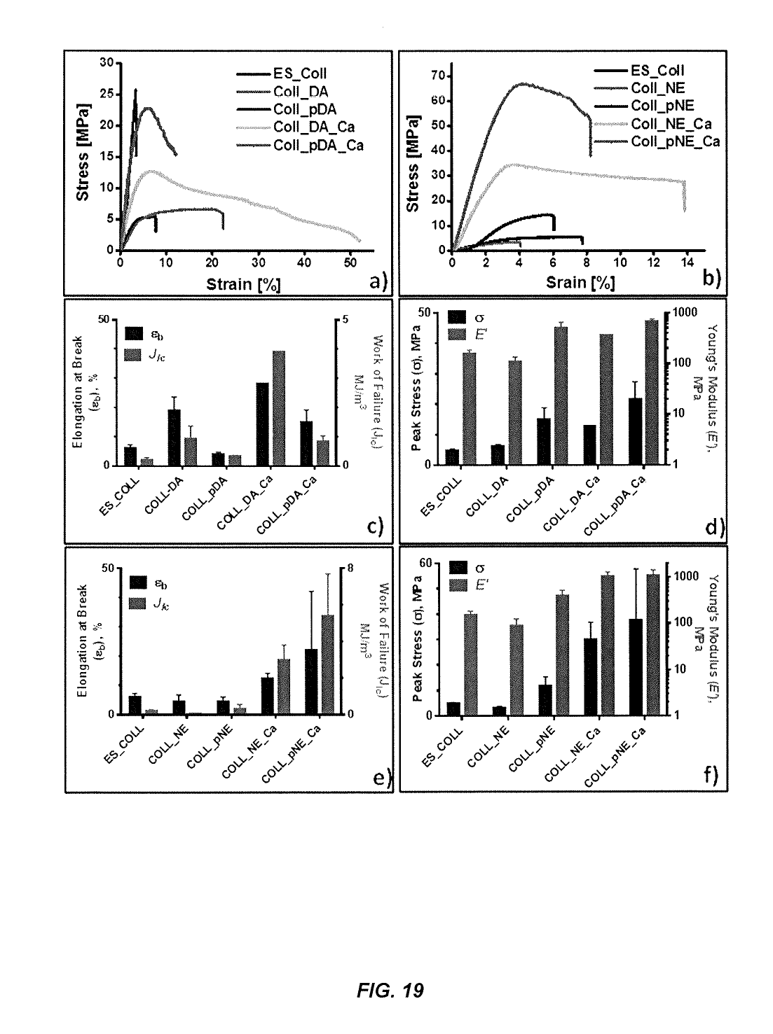

Next we investigated the effect of incorporating catecholamines/Ca²⁺ and subsequent mineralization and crosslinking on the mechanical properties of collagen mats. Typical stress-strain curves for various collagen mats measured by uniaxial tensile testing are shown in FIGS. 19A and 19B. We determined peak stress (σ), elongation at break (ε_{b}), Young's modulus (E′) and work of failure (J_{lc}) from the stress-strain curves (Table 6). Detailed statistical analyses of the mechanical properties for various mats are presented in Table 7. For ES_Coll, a brittle-like behavior was observed with a σ value of 4.9±0.5 MPa and ε_{b} of 6.0±1.3%. A tensile modulus of 156.7±29 MPa and mechanical toughness of 0.22±0.07 MJm⁻³ was estimated from the stress-strain curves (FIG. 19A).

TABLE 6
Mechanical properties of electrospun collagen mats prepared under various
conditions. Significance values: *, p ≤ 0.05; **, p < 0.01; ***, p < 0.001;
****, p < 0.0001 and ns, p > 0.05 by t-test or 1-way ANOVA.

| | | | Young's | Work of |
|---|---|---|---|---|
| | Peak Stress | Failure | Modulus | Failure |
| Sample | (MPa) | Strain (%) | (MPa) | (MJ/m³) |
| ES_Coll | 4.9 ± 0.5 | 6.0 ± 1.3 | 156.7 ± 29 | 0.22 ± 0.07 |
| Coll_DA | 6.3 ± 0.5^{ns} | 19.2 ± 4.4** | 108.8 ± 27.3^{ns} | 0.93 ± 0.44** |
| Coll_pDA | 15.1 ± 3.8* | 4.1 ± 0.6^{ns} | 512.6 ± 136.9** | 0.334 ± 0.03^{ns} |
| Coll_DA_Ca | 12.8 | 28.1 | 359.6 | 3.9 |
| Coll_pDA_Ca | 21.7 ± 5.7*** | 15.1 ± 4.1* | 675.8 ± 81.2**** | 0.83 ± 0.00* |
| Coll_NE | 3.36 ± 0.5^{ns} | 4.5 ± 2.3^{ns} | 88.4 ± 36.1^{ns} | 0.07 ± 0.03^{ns} |
| Coll_pNE | 11.8 ± 3.6^{ns} | 4.5 ± 1.6^{ns} | 400.5 ± 126^{ns} | 0.32 ± 0.25^{ns} |
| Coll_NE_Ca | 29.9 ± 6.9**** | 12.4 ± 1.8** | 1065 ± 240**** | 3.0 ± 0.8**** |
| Coll_pNE_Ca | 37.6 ± 20.3**** | 22.2 ± 20**** | 1070 ± 367*** | 5.36 ± 2.3**** |

Addition of 10% DA, increased the elastic properties of the mats as indicated by an increase in ε_{b} and J_{lc} values (FIG. 19A). After ADM, a brittle-like behavior was observed as indicated by significant increase in σ (15.1±3.8 MPa) or E′ (512.6±136.9 MPa) and low ε_{b} values (4.1±0.6%, FIGS. 19C, 19D). However, incorporation of Ca²⁺ into the dope solution containing DA had profound influence on tensile properties, as the mats prepared from the solution displayed remarkable increase in σ, E′ and ε_{b} values (FIGS. 19C, 19D). It was interesting to note that the tensile properties of Coll_DA_Ca approached the values obtained for Coll_pDA, confirming that the presence of Ca²⁺ in the dope solution triggered the oxidative polymerization of DA. For Coll_pDA and Coll_pDA_Ca no significant difference in σ and E′ values (p>0.05) were observed whereas considerable differences were apparent in the elastic properties. These results suggest that the formation CaCO₃ increased the toughness of the composites without affecting tensile stiffness and strength of polycatecholamines. crosslinked collagen. A similar trend was observed in the case of mats containing NE (FIGS. 19B, 19E, 19F). In fact, the influence of Ca²⁺ on the mechanical properties of the mats containing NE was so remarkable that E′ values approached sub-GPa with higher elasticity/toughness than observed for mats containing DA (Table 6). These results suggest that incorporation of Ca²⁺ into catecholamines containing collagen and subsequent mineralization increased the elastic properties with concomitant enhancement in mechanical strength and stiffness. Such remarkable enhancement in both stiffness and toughness of the composites compared to pristine polymers could not be achievable from the composite mats prepared by direct mixing of minerals and polymers reported by others. See, Fujihara 2005; Gao 2013; Kim 2005; Frohbergh 2012; Gupta 2009; Kharaziha 2013; Wutticharoenmongkol 2006.

TABLE 7
Statistical comparison of the four mechanical properties determined for various
electrospun mats. Significance values: *, p ≤ 0.05; **, p < 0.01; ***, p < 0.001;
****, p < 0.0001 and ns, p > 0.05 by t-test or 1-way ANOVA.

| Scaffold Type | | ES_Coll | Coll_DA | Coll_pDA | Coll_DA_Ca | Coll_pDA_Ca |
|---|---|---|---|---|---|---|
| σ, MPa | ES_Coll | — | ns | * | ns | *** |
| | Coll_DA | ns | — | * | ns | *** |
| | Coll_pDA | * | * | — | ns | ns |
| | Coll_DA_Ca | ns | ns | ns | — | * |
| | Coll_pDA_Ca | *** | *** | ns | * | — |
| E′, MPa | ES_Coll | — | ns | ** | * | **** |
| | Coll_DA | ns | — | *** | * | **** |
| | Coll_pDA | ** | *** | — | ns | ns |
| | Coll_DA_Ca | * | * | ns | — | ** |
| | Coll_pDA_Ca | **** | **** | ns | ** | — |
| ε_{b}, % | ES_Coll | — | ** | ns | **** | * |
| | Coll_DA | ** | — | *** | ** | ns |
| | Coll_pDA | ns | *** | — | **** | ** |
| | Coll_DA_Ca | **** | ** | **** | — | ** |
| | Coll_pDA_Ca | * | ns | ** | ** | — |
| J_{lc}, MJm⁻³ | ES_Coll | — | ** | ns | **** | * |
| | Coll_DA | ** | — | * | **** | ns |
| | Coll_pDA | ns | * | — | **** | ns |
| | Coll_DA_Ca | **** | **** | **** | — | **** |
| | Coll_pDA_Ca | * | ns | ns | **** | — |
| Scaffold_Type | | ES_Coll | Coll_NE | Coll_pNE | Coll_NE_Ca | Coll_pNE_Ca |
| σ, MPa | ES_Coll | — | ns | ns | **** | **** |
| | Coll_NE | ns | — | ns | **** | **** |
| | Coll_pNE | ns | ns | — | *** | **** |
| | Coll_NE_Ca | **** | **** | *** | — | ns |
| | Coll_pNE_Ca | **** | **** | **** | ns | — |
| E′, MPa | ES_Coll | — | ns | ns | **** | *** |
| | Coll_NE | ns | — | ns | **** | **** |
| | Coll_pNE | ns | ns | — | *** | ** |
| | Coll_NE_Ca | **** | **** | *** | — | — |
| | Coll_pNE_Ca | *** | **** | ** | ns | ns |
| ε_{b}, % | ES_Coll | — | ns | ns | ** | **** |
| | Coll_NE | ns | — | ns | *** | **** |
| | Coll_pNE | ns | ns | — | *** | **** |
| | Coll_NE_Ca | ** | *** | *** | — | **** |
| | Coll_pNE_Ca | **** | **** | **** | **** | — |
| J_{lc}, MJm⁻³ | ES_Coll | — | ns | ns | **** | **** |
| | Coll_NE | ns | — | ns | **** | **** |
| | Coll_pNE | ns | ns | — | **** | **** |
| | Coll_NE_Ca | **** | **** | **** | — | ns |
| | Coll_pNE_Ca | **** | **** | **** | ns | — |

Abbreviations:
σ; Peak Stress,
E′; Young's Modulus,
ε_{b}; Elongation at Break,
J_{lc}; Work of Failure

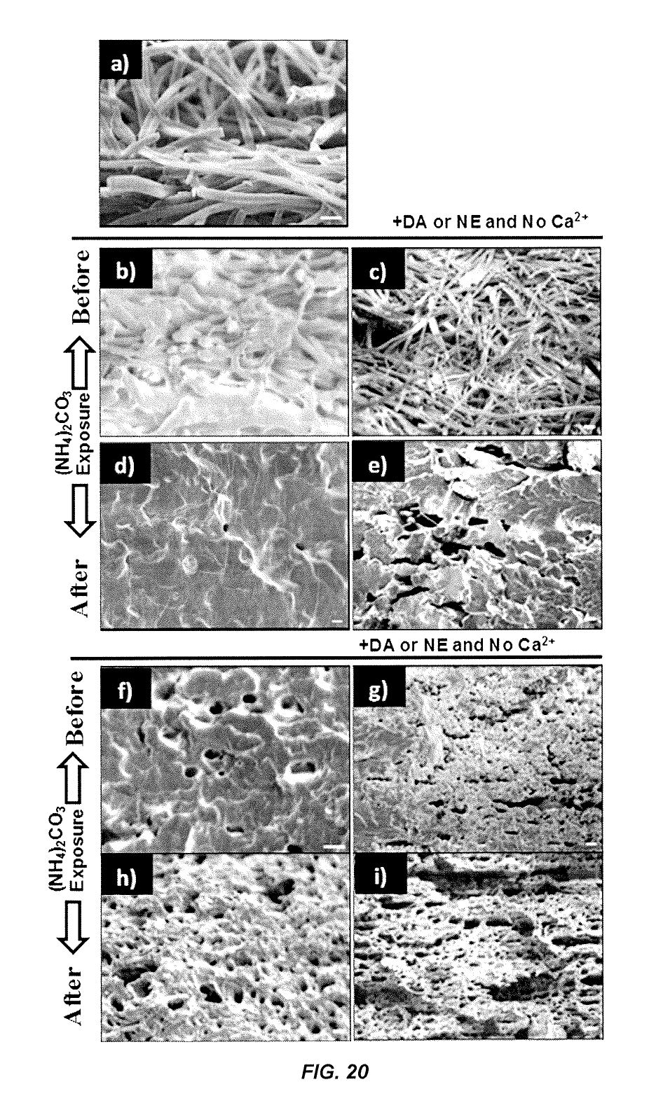

To shed more insight into the mechanical properties, we imaged the fracture surface of various mats by SEM. The deformed structure of ES_Coll and Coll_DA and Coll_NE mats contained numerous collagen fibrils (FIGS. 20A-20C). The deformed surface of Coll_pDA contained numerous fracture steps indicating weak resistance to the applied stress (FIG. 20D), confirming the brittle-like behavior. See, Tian 2007. For Coll_pNE due to the formation of welded junctions and smooth polycatecholamine coating, the fractured surface appeared as fiber-reinforced composite structures (FIG. 20E). However, incorporation of Ca²⁺ considerably altered the fracture morphology as detected by significant increase in crack branching and river-like lines on the fractured surface of Coll_DA_Ca and Coll_NE_Ca mats (FIGS. 20F, 20G). The roughness of the surface increased significantly for Coll_pDA_Ca and Coll_pNE_Ca mats (FIGS. 20H, 20I). The high abundance of welded junctions and coating formed in the presence of Ca²⁺ may account for the increase in the mechanical properties of the mats while the formation CaCO₃ particles increase the roughness and prevents the crack propagation thus enhancing the toughness of the composites. See, Tian 2007.

### Example 8. Photoluminescent Properties of Composite Nanofiber Polymer Product

Photoluminescence Properties of Nanofibrous Scaffolds.

Fluorescence microscopy was performed using a confocal microscope (Zeiss LSM 800 Airyscan, Carl Zeiss Microimaging Inc., NY, USA) equipped with 405, 488, 561 and 640 nm excitation lasers. A Plan-Apochromat 63×/1.4 Oil DIC M27 objective lens (Carl Zeiss) was used and the confocal pinhole was set to 1 Airy unit for the green channel and other channels were adjusted to the same optical slice thickness. Images from different sample groups were acquired using identical laser percentage and digital gain for each laser channel. Zen 2.1 lite lite imaging software (Carl Zeiss) was used for the figure preparation.

Photoluminescence Properties of ES Collagen Mats.

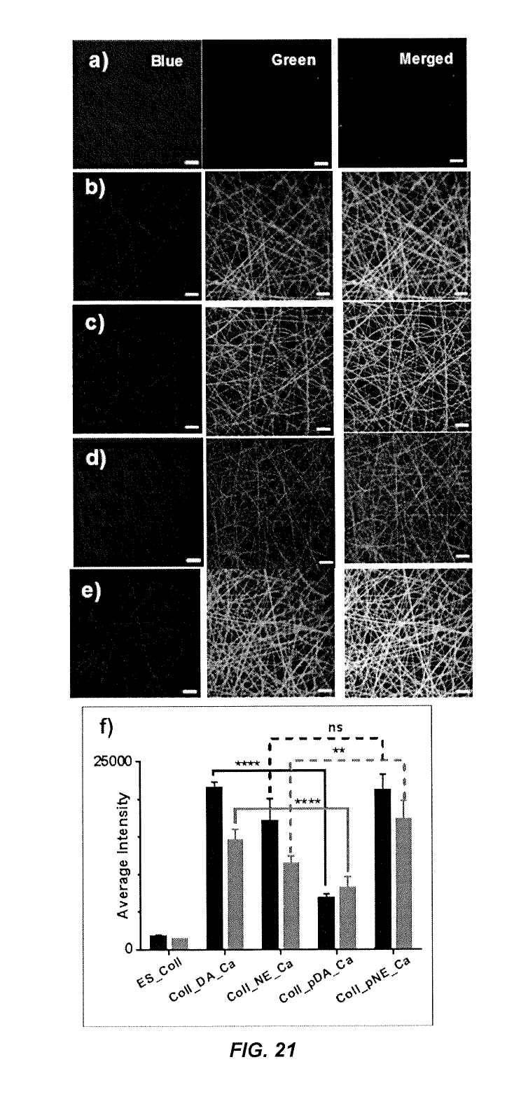

Recent studies have shown that addition of cationic polyethylenimine (PEI) during alkaline oxidative polymerization resulted in PEI-pDA copolymer with enhanced fluorescence emission properties. See, Zhao 2015. Therefore, we investigated the optical properties of catecholamines crosslinked mats by fluorescence microscopy and spectroscopy. Pristine ES_Coll mats displayed a weak blue emission whereas, mats containing polycatecholamines displayed intense blue and green emission colors (FIGS. 21A-21C). In particular, the fluorescence intensity was stronger at the soldered junctions. For collagen mats containing dopamine, mineralization affected the fluorescent properties as substantial decrease in both blue and green fluorescence intensities was observed Coll_pDA_Ca (FIGS. 21B, 21D). However, for mats containing NE, mineralization did not alter the blue fluorescence intensity whereas a slight increase in the green fluorescence intensity was observed for Coll_pNE_Ca (FIGS. 21C, 21E). Quantitative estimation of the average fluorescence intensities further confirmed the above results from images (FIG. 21F).

The origin for the differences in photoluminescent properties remain unclear, it is likely that the two polycatecholamines may interact differently with the inorganic minerals formed after ADM. The development of composite structures with excellent mechanical, photoluminescent and biological (see below) properties can function as non-invasive real-time probes as well for quantifying the scaffold degradation patterns and tissue integration without sacrificing the animals. See, Yang 2009; Xie 2014; Li 2016. and would open a new paradigm for the implementation of smart multifunctional scaffolds for tissue engineering and regenerative medicine.

### Example 9. Cell Culture with Composite Nanofiber Polymer Products

Dulbecco's modified eagle's medium (DMEM), nutrient mixture F-12 (HAM), antibiotics (cat# A5955), and Hoechst dye were obtained from Sigma-Aldrich (Singapore). Human fetal osteoblast cells (hFob) were obtained from the American type culture collection (ATCC, Arlington, Va.). For cell culture, fetal bovine serum (FBS) and trypsin-EDTA were purchased from GIBCO Invitrogen, USA.

Human Fetal Osteoblasts (hFob) Cell Culture.

The hFob cells were cultured in DMEM/F12 medium (1:1) supplemented with 10% FBS and cocktail antibiotics in 75 cm² cell-culture flasks. The hFob cells were incubated at 37° C. in humidified CO₂ incubator for 1 week and fed with fresh medium every 3 days. Cells were harvested after 3^{rd} passage using trypsin-EDTA treatment and replated after cell counting with trypan blue using haemocytometer. For cell seeding, the nanofibrous scaffolds were collected on 15 mm coverslips and sterilized under UV light for 1 h. The scaffolds were then placed in 24-well plates with stainless steel rings to prevent the lifting up of the scaffolds. The scaffolds were then washed with 10 mM PBS (pH 7) thrice for 15 min to remove the residual solvent and finally soaked in complete media overnight. The hFob cells were seeded at a density of 1×10⁴ cells well⁻¹ on COLL_DA_Ca, COLL_pDA_Ca, COLL_NE_Ca and COLL_pNE_Ca scaffolds. ES_Coll and TCP served as positive controls.

Cells Viability Analysis:

Cell growth and proliferation on the different collagen scaffolds was tested by the colorimetric MTS (3-(4,5-dimethylthiazol-2-yl)-5-(3-carboxymethoxyphenyl)-2 (4-sulfophenyl)-2H-tetrazolium, inner salt) assay. The cellular scaffolds were rinsed with PBS and incubated with MTS reagent in serum-free medium for 3 h. Metabolically active cells reacted with the tetrazolium salt present in MTS reagent producing a soluble purple formazan dye. The aliquots were then transferred into 96-well plates and the absorbance of the samples was read at 490 nm using a spectrophotometric plate reader (FLUOstar OPTIMA, BMG Lab Technologies, Germany).

Calcein FDA is a cell penetrating dye and readily cleaved by the intracellular esterases present in live cells, thus producing fluorescent calcein. After 3, 6 and 9 days of cell growth, the complete medium was removed from the 24-well plates and the cells were fed with DMEM medium. The scaffolds were then incubated with 20 μl of the CMFDA dye (25 μM in medium) for 2 h at 37° C. Thereafter the CMFDA medium was removed and 1 ml of complete medium was added to the cells and incubated overnight. Before imaging by confocal microscopy, the cells were treated with Cytiva Cell Health Reagent for 1 h to report cell count, nuclear morphology and cell viability/treated with Hoechst dye and propidium iodide for 1 h to visualize all nuclei and identify dead cells. An automated microscope IN Cell Analyzer 2200 (GE Healthcare) was used to randomly scan 9 fields/cell samples using ×10 objectives. Quantitative live/dead cell analysis of the acquired images was performed with IN Cell Investigator software (GE Healthcare). Confocal images and z-stacks were acquired with 405, 488 and 561 nm lasers excitation using Zeiss LSM800 Airyscan a Plan-Apochromat 40×/1.3 oil immersion objective lens.

To visualize the morphologies and cytoskeletal structures of cells growing on different scaffolds, cells were fixed with 4% (v/v) formaldehyde at day 3, 6 or 9. After fixation, cells were stained with Far-Red fluorescently labelled cytoplasmic dye, phalloidin (Molecular Probes®) and Hoechst. Confocal images and z-stacks were acquired with 405, 488 and 640 nm lasers excitation using Zeiss LSM800 Airyscan a Plan-Apochromat 40×/1.3 oil immersion objective lens. Confocal images were prepared using Zen 2.1 lite imaging software (Carl Zeiss).

Results.

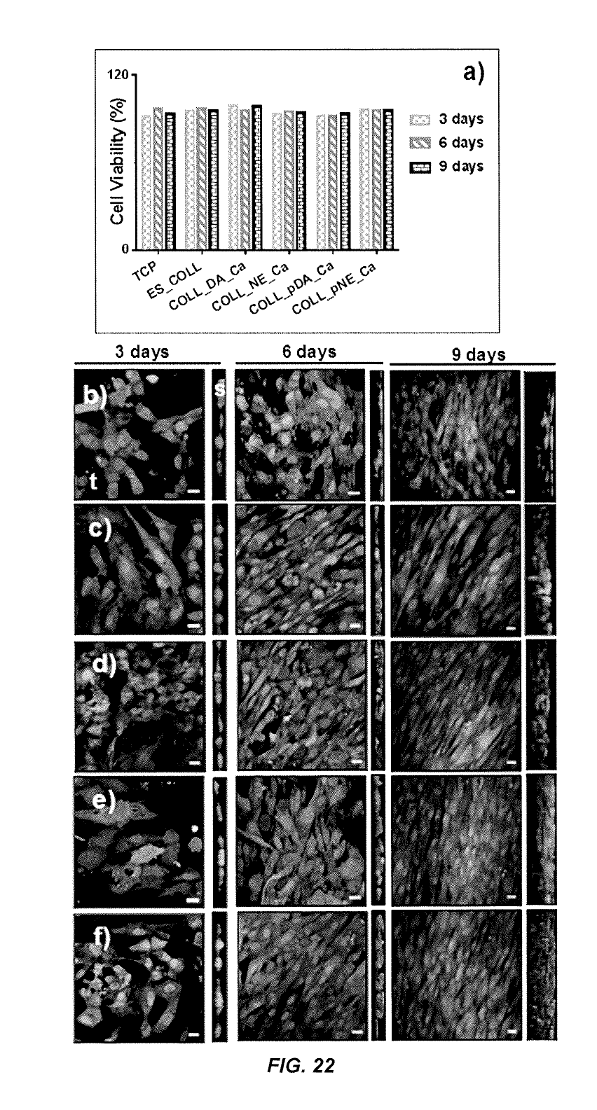

The cytotoxicity of the scaffolds for hFob cells was evaluated by calcein fluorescein diacetate (Calcein FDA) assay. Cells were seeded on ES_Coll, Coll_DA_Ca, Coll_NE_Ca, Coll_pDA_Ca and Coll_pNE_Ca mats. Cells seeded on the tissue culture plate (TCP) served as control. After 3, 6 and 9 days post seeding (p.s.) of hFob on various scaffolds, cells were stained with caclein FDA and PI to obtain semi-quantitative information live cell/dead cell ratio. All the scaffolds displayed no discernable cytotoxicity to hFob cells as the % live cells remained >90% at various time points (FIG. 22A), thus confirming the excelent biocompatibility of scaffolds for hFob.

Confocal images of the cells cultured on various scaffolds are shown in FIGS. 22B-22F at three different time points. Interestingly, an increased cell populations and spreading was observed in the Coll_pDA/pNE_Ca mats (FIGS. 22E, 22F), as indicated by increased staining of CellTracker Green CMFDA and nuclei (blue) after 9 days p.s. These results suggest more cell spreading on Coll_pDA/pNE_Ca mats than on ES_Coll. Confocal z-stack imaging allowed us to examine the level of cell penetration into various collagen mats. A time-dependent increase in the cell penetration was observed on all the mats with markedly increased penetration in Coll_pDA/pNE_Ca mats. Together with xy-scans, these results suggest increased cell spreading and active cell migration on mineralized scaffolds.

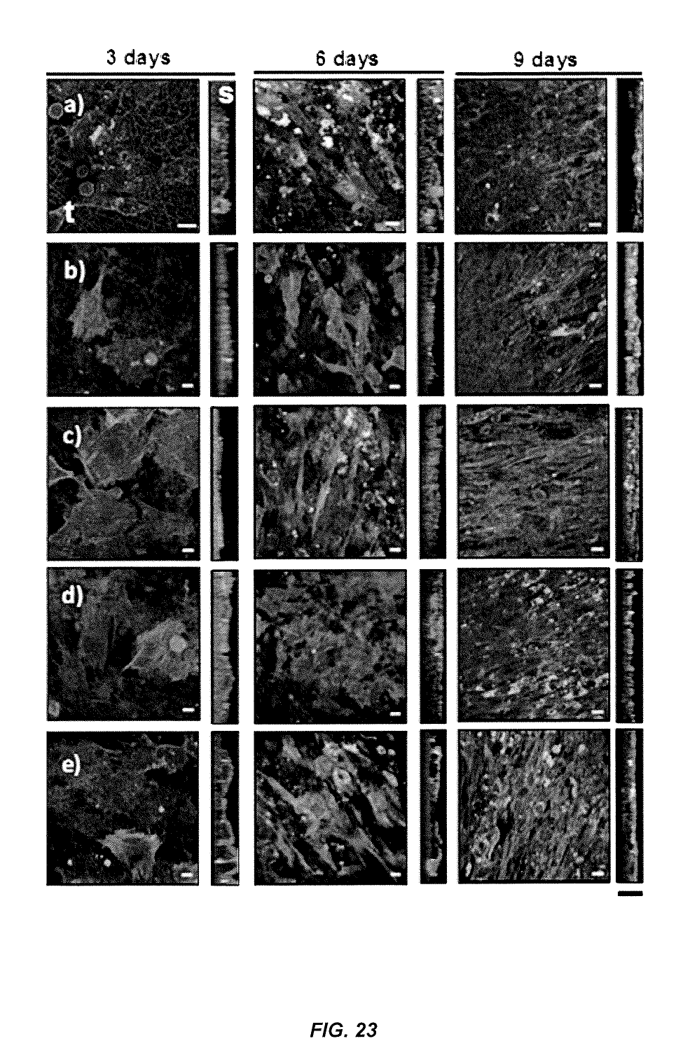

To obtain better insight into the cell infiltration into the scaffolds, hFob cells were stained with Far-Red cytoplasmic dye and imaged from surface as well throughout the scaffold depth (FIGS. 23A-23F). Top-view images indicated an enhanced cell growth on Coll_pDA_Ca and Coll_pNE_Ca mats, consistent with the previous results. Side-view images suggested a time-dependent increase in cell infiltration on all electrospun scaffolds, as indicated by increase in far-red staining with increasing p.s. As was observed with the CM-FDA results, the mineralized mats (Coll_pDA_Ca and Coll_pNE_Ca) displayed enhanced cell infiltration with a maximum depth of 30 μm at 9 days p.s.

Osteoblasts Proliferation on ES Collagen Matrices:

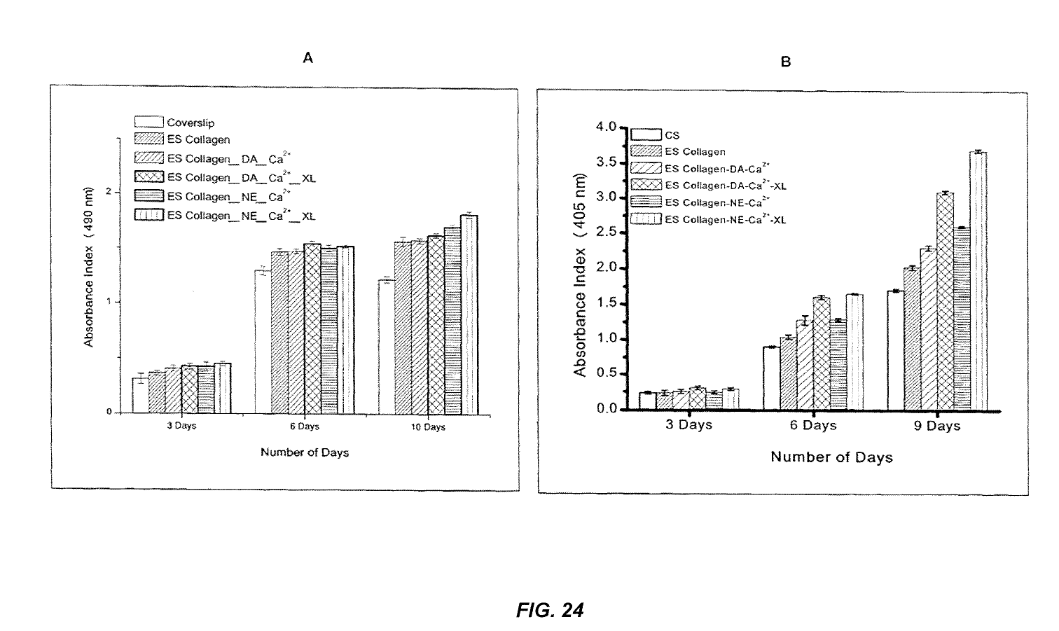

To study the effect of calcium doping, the human fetal osteoblasts (hFOB) cell proliferation of the ES collagen containing Ca²⁺-loaded was determined by MTS assay. FIG. 24A compares the proliferation rates of hFOB under various conditions. The incorporation of catecholamines-Ca²⁺ in the dope solution increased the cell proliferation rates compared to the proliferation rate on pristine collagen matrix or glass coverslips and the effect was remarkable at an extended period. These results indicated that the crosslinked collagen matrices displayed enhanced cellular response compared to pristine ES collagen.

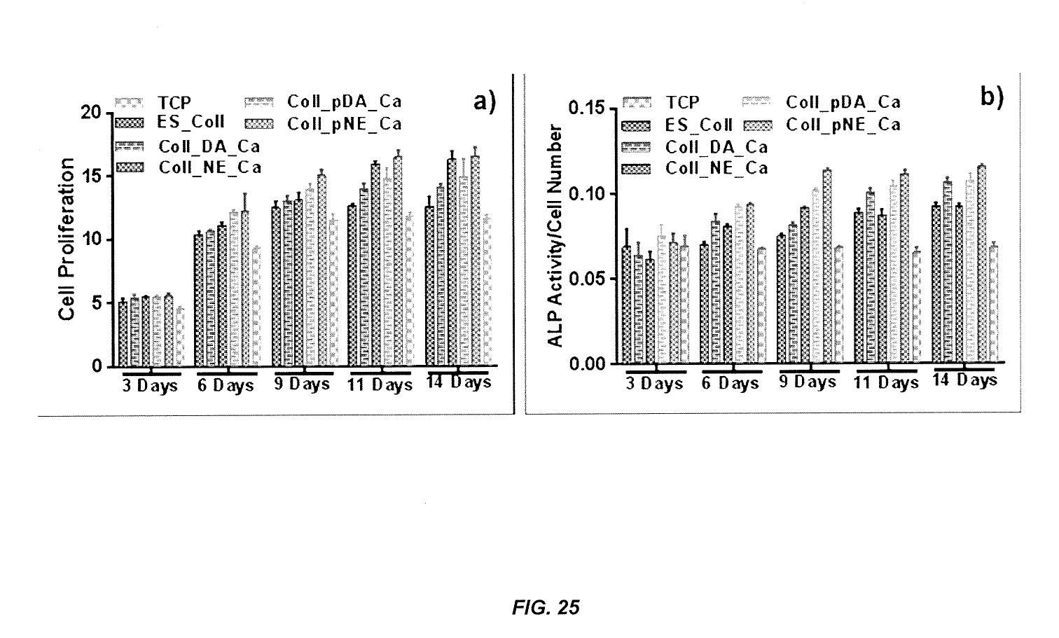

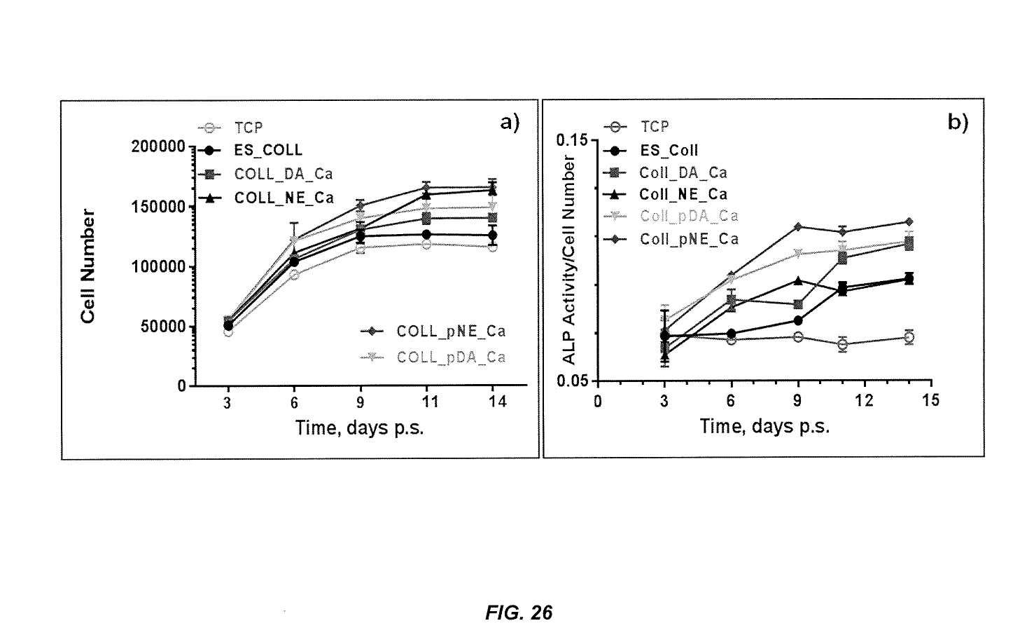

Data was expressed in cell proliferation ratio after dividing cell number at each time point with initial cell density. As shown in FIG. 25A, all the electrospun scaffolds supported cell attachment and proliferation until 9 days p.s., indicated by the increase in cell proliferation values, and levels of after 11 and 14 days p.s. Since collagen provides active cell recognition sites for hFob adhesion and proliferation, we observed higher rate of cell proliferation on ES scaffolds than TCP. Among the various collagen mats, no statistically significant differences in hFob proliferation were observed at 3 days p.s. (Table 8). However, catecholamines loaded mineralized mats (Coll_pDA/pNE_Ca), displayed increased proliferation as early as 6 days p.s. As the time progress, cells cultured on all the catecholamine/Ca²⁺ mats (Coll_DA/NE_Ca and Coll_pDA/pNE_Ca) showed significantly pronounced metabolic activity compared to ES_Coll (FIG. 26A). See, Gupta 2009; Prabhakaran 2009.

TABLE 8
Statistical comparison of the cell proliferation data for
various collagen scaffolds and TCP, determined by MTS assay.
Significance values: *, p ≤ 0.05; **, p < 0.01; ***,
p < 0.001; ****, p < 0.0001 and ns, p > 0.05
by t-test or 1-way ANOVA.

| Scaffold Type | | TCP | ES_Coll | Coll_DA_Ca |
|---|---|---|---|---|
| 3 | TCP | ns | ns | * |
| Days | ES_Coll | ns | ns | ns |
| | Coll_DA_Ca | * | ns | ns |
| | Coll_pDA_Ca | ** | ns | ns |
| | Coll_NE_Ca | ** | ns | ns |
| | Coll_pNE_Ca | ** | * | ns |
| 6 | TCP | ns | **** | **** |
| Days | ES_Coll | **** | ns | ns |
| | Coll_DA_Ca | **** | ns | ns |
| | Coll_pDA_Ca | **** | * | ns |
| | Coll_NE_Ca | **** | ns | ns |
| | Coll_pNE_Ca | **** | ns | ns |
| 9 | TCP | ns | **** | **** |
| Days | ES_Coll | **** | ns | ns |
| | Coll_DA_Ca | **** | ns | ns |
| | Coll_pDA_Ca | **** | ns | ns |
| | Coll_NE_Ca | **** | ** | ** |
| | Coll_pNE_Ca | **** | **** | **** |
| Scaffold Type | | Coll_pDA_Ca | Coll_NE_Ca | Coll_pNE_Ca |
| 3 | TCP | ** | ** | ** |
| Days | ES_Coll | * | ns | * |
| | Coll_DA_Ca | ns | ns | ns |
| | Coll_pDA_Ca | ns | ns | ns |
| | Coll_NE_Ca | ns | ns | ns |
| | Coll_pNE_Ca | ns | ns | ns |
| 6 | TCP | **** | **** | **** |
| Days | ES_Coll | * | ns | ns |
| | Coll_DA_Ca | ns | ns | ns |
| | Coll_pDA_Ca | ns | ns | ns |
| | Coll_NE_Ca | ns | ns | ns |
| | Coll_pNE_Ca | ns | ns | ns |
| 9 | TCP | **** | **** | **** |
| Days | ES_Coll | ns | ** | **** |
| | Coll_DA_Ca | ns | ** | **** |
| | Coll_pDA_Ca | ns | ns | **** |
| | Coll_NE_Ca | ns | ns | ** |
| | Coll_pNE_Ca | **** | ** | ns |

Among the two catecholamines, NE-loaded mats promoted higher proliferation than mats containing DA. Analysis of calcein FDA and MTS results revealed the following conclusions: i) Compared to TCP, a unanimous increase in the hFob proliferation was observed for the mats at all time points. ii) Incorporation of Ca²⁺ into the catecholamines loaded collagen and subsequent exposure to (NH₄)₂CO₃ promote enhanced cell spreading and proliferation, iii) Among the two catecholamines, cell proliferation was more pronounced in the NE incorporated mats i.e., COLL_NE_Ca (p<0.0001 vs. Coll_DA_Ca at day 11 p.s.) and COLL_pNE_Ca (p<0.0001 Coll_pDA_Ca day 11 p.s.). These results suggested that the polycatechoalmine-CaCO₃ composite structures are non-cytotoxic for the hFob and stimulate the adherence and proliferation of bone cells and the properties can be modulated by appropriate choice of catecholamines.

### Example 10. Osteoconductive Properties of Composite Nanofiber Polymer Product

Alkaline Phosphatase (ALP) Activity of the Cells:

The expression of alkaline phosphatase activity of the cells was used to estimate their bone-forming ability on the different scaffolds. The cell-scaffold constructs were washed with PBS for 15 min and ALP (Sigma, Singapore) reagent was added. After 1 hour of incubation the reaction was stopped using 2N NaOH. This assay uses p-nitrophenyl phosphate (pNPP) as a colourless phosphatase organic ester substrate which turns yellow when dephosphorylated, or in other words, when catalyzed by ALP forming p-nitrophenol and phosphate. The yellow colour product was aliquot in 96-well plate and reaction absorbance was measured at 405 nm using a microplate reader.

Alkaline phosphatase (ALP) is an important enzyme responsible for the mineral nucleation by supplying free phosphate ions through lysis of organic phosphates and its expression is associated with cell differentiation. ALP activity of hFob cells seeded on various scaffolds was estimated using an alkaline phosphate yellow liquid substitute system for enzyme-linked immunosorbent assay (ELISA) (Sigma Life Science, USA). In this assay, ALP catalyses the hydrolysis of colourless p-nitro phenyl phosphate (PNPP) to a yellow product p-nitrophenol and phosphate. At 3, 6, 9, 11 and 14 days p.s., the medium was removed from the 24-well plates and the scaffolds were washed thrice with PBS. The scaffolds were then incubated with 400 μl of PNPP solution for 30 min. The reaction was dragged to completion by adding 200 μl of 2 M NaOH solution. The resultant yellow coloured solution was then pipetted out into the 96-wells plates and the absorbance for different scaffolds was read at 405 nm in micro-plate reader. To present the ALP activity of the cells at different time points, the ALP activity was normalized with cell number as a marker for bone formation.

The cultured hFob cells also showed enhanced differentiation, when seeded onto the catecholamines-Ca²⁺ doped ES fiber mats after exposure to gaseous ammonia (FIG. 24B). In particular, calcium containing DA-/NE-loaded mats after exposed to gaseous ammonia increased the ALP activity by >1.5× compared to pristine collagen mats.

ALP is a key component of bone matrix vesicles that catalyze the cleavage of organic phosphate esters and play a significant role in the formation of bone mineral, and is an early indicator of immature osteoblast activity. See, Yang 2009. ALP activity is also a marker of early osteoblastic differentiation and commitment of the stem cells towards the osteoblastic phenotype. See, Xie 2014. We, therefore, examined the osteogenic differentiation of the mats by measuring the ALP activity, using a colorimetric pNPP assay on days 3, 6, 9, 11 and 11 days post-seeding. The ALP activity was normalized by cell number and shown in FIG. 25B. No significant variation in the ALP activity was observed for all the investigated collagen scaffolds on 3 days p.s. (FIG. 25B and Table 9A). However, compared to the pristine collagen or TCP, the ALP activity increased dramatically for all the catecholamine/Ca²⁺ incorporated scaffolds (p<0.0001) after 6 day p.s. of hFob. The hFob differentiated stably for an extended period of time when cultured on Coll_pDA/pNE_Ca mats after 6-14 days p.s. than cells cultured on Coll_DA/NE_Ca, suggesting that mineralized scaffolds promote cell differentiation as well (FIG. 26B). Among the two catecholamines, a significantly higher ALP activity was observed for cells cultured on COLL_pNE_Ca than on COLL_pDA_Ca mats during early stages (Table 9A), confirming increased osteogenic differentiation potential of oxidative products of norepinephrine. Together with the cell proliferation assays, these results demonstrate that COLL_pDA/pNE_Ca mats are superior in terms of osteoblasts cell growth, penetration and differentiation.

TABLE 9A
Statistical comparison of the ALP activity data for various
collagen scaffolds and TCP. Significance values: *, p ≤ 0.05;
**, p < 0.01; ***, p < 0.001; ****, p < 0.0001 and ns,
p > 0.05 by t-test or 1-way ANOVA.

| Scaffold Type | | TCP | ES_Coll | Coll_DA_Ca |
|---|---|---|---|---|
| 3 | TCP | ns | ns | ns |
| Days | ES_Coll | ns | ns | ns |
| | Coll_DA_Ca | ns | ns | ns |
| | Coll_pDA_Ca | ns | * | ns |
| | Coll_NE_Ca | ns | ns | ns |
| | Coll_pNE_Ca | ns | ns | ns |
| 6 | TCP | ns | ** | **** |
| Days | ES_Coll | ** | ns | **** |
| | Coll_DA_Ca | **** | **** | ns |
| | Coll_pDA_Ca | **** | **** | **** |
| | Coll_NE_Ca | **** | **** | ns |
| | Coll_pNE_Ca | **** | **** | **** |
| 9 | TCP | ns | **** | **** |
| Days | ES_Coll | **** | ns | **** |
| | Coll_DA_Ca | **** | **** | ns |
| | Coll_pDA_Ca | **** | **** | **** |
| | Coll_NE_Ca | **** | **** | **** |
| | Coll_pNE_Ca | **** | **** | **** |
| Scaffold Type | | Coll_pDA_Ca | Coll_NE_Ca | Coll_pNE_Ca |
| 3 | TCP | ns | ns | ns |
| Days | ES_Coll | * | ns | ns |
| | Coll_DA_Ca | ns | ns | ns |
| | Coll_pDA_Ca | ns | ns | ns |
| | Coll_NE_Ca | ns | ns | ns |
| | Coll_pNE_Ca | ns | ns | ns |
| 6 | TCP | **** | **** | **** |
| Days | ES_Coll | **** | **** | **** |
| | Coll_DA_Ca | **** | ns | **** |
| | Coll_pDA_Ca | ns | **** | ns |
| | Coll_NE_Ca | **** | ns | **** |
| | Coll_pNE_Ca | ns | **** | ns |
| 9 | TCP | **** | **** | **** |
| Days | ES_Coll | **** | **** | **** |
| | Coll_DA_Ca | **** | **** | **** |
| | Coll_pDA_Ca | ns | **** | **** |
| | Coll_NE_Ca | **** | ns | **** |
| | Coll_pNE_Ca | **** | **** | ns |

Detection of Cells Mineralization on Nanofibrous Scaffolds:

Undifferentiated Mesenchymal Stem Cells (MSCs) have no extracellular calcium deposits but once the MSCs got differentiated into osteoblasts there is more accumulation of calcium in-vivo and in-vitro. In other words the extent of calcium deposit can be taken as an indication for the successful differentiation of the osteoblast cells. To visualize the mineralization over the scaffolds due to osteoblast differentiation, Alizarin Red S (ARS) staining was used. For ARS staining, at each time point (3rd day, 6th day and 9th Day), after discarding the medium, scaffolds were washed three times with PBS followed by treatment to 70% ethanol for 1 h and washed again with distilled water twice. After staining the cells over the scaffold with 40 mM ARS for 30 mins, the microscopic images were taken to show the extent of calcium deposit over the scaffolds.

Alizarin Red-S (ARS) Staining Assay.

ARS staining was used to qualitatively and quantitatively detect the extent of mineralization on various scaffolds. ARS is a dye that selectively binds to the calcium salts and used for the calcium mineral histochemistry. The nanofibrous scaffolds with the hFob cells were first washed thrice using PBS and then the cells were fixed by treating them to 70% ethanol for 1 h. The cellular constructs were then washed thrice with DI water followed by staining with ARS (40 mM) for 20 min at room temperature. The scaffolds were then washed with DI water several times and visualized under optical microscope. For quantitative assessment, the stain was eluted with 10% cetylpyridinium chloride for 60 min. The absorbance of solution was recorded at 540 nm on Tecan plate reader.

Alizarin Red Staining:

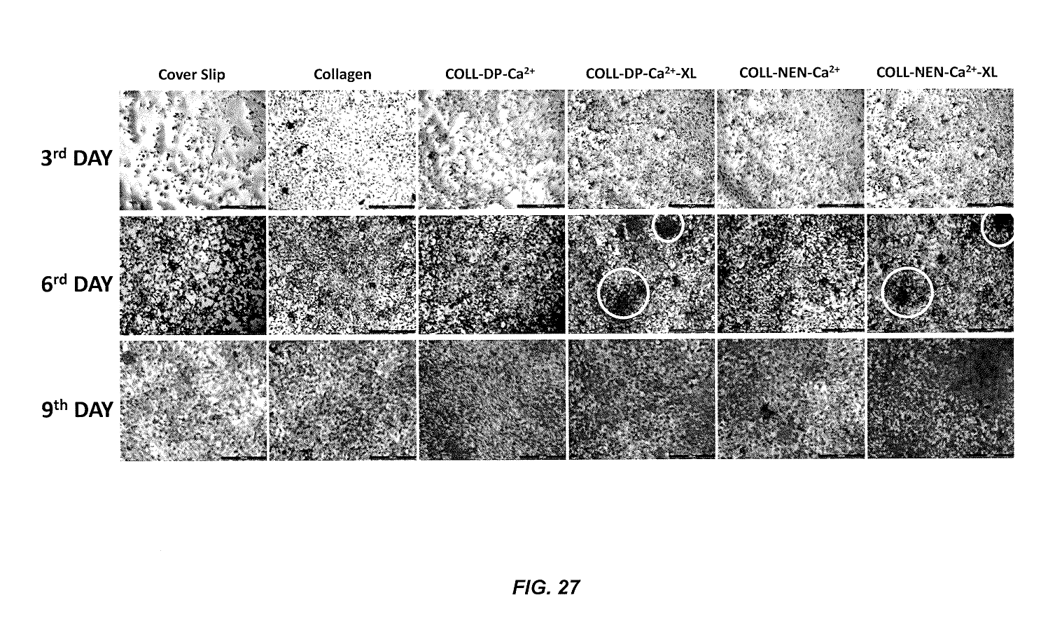

To determine extracellular mineral deposition, ARS staining was used. FIG. 27 shows optical microscopy of the scaffolds stained after seeding of hFOB at various intervals. The as-spun mats containing catecholamines-Ca²⁺ showed higher mineral deposition than pristine collagen. Consistent with the cell differentiation and proliferation assays, catecholamines-Ca²⁺ doped mats that exposed to gaseous ammonia showed the highest deposition on days 3 and 6.

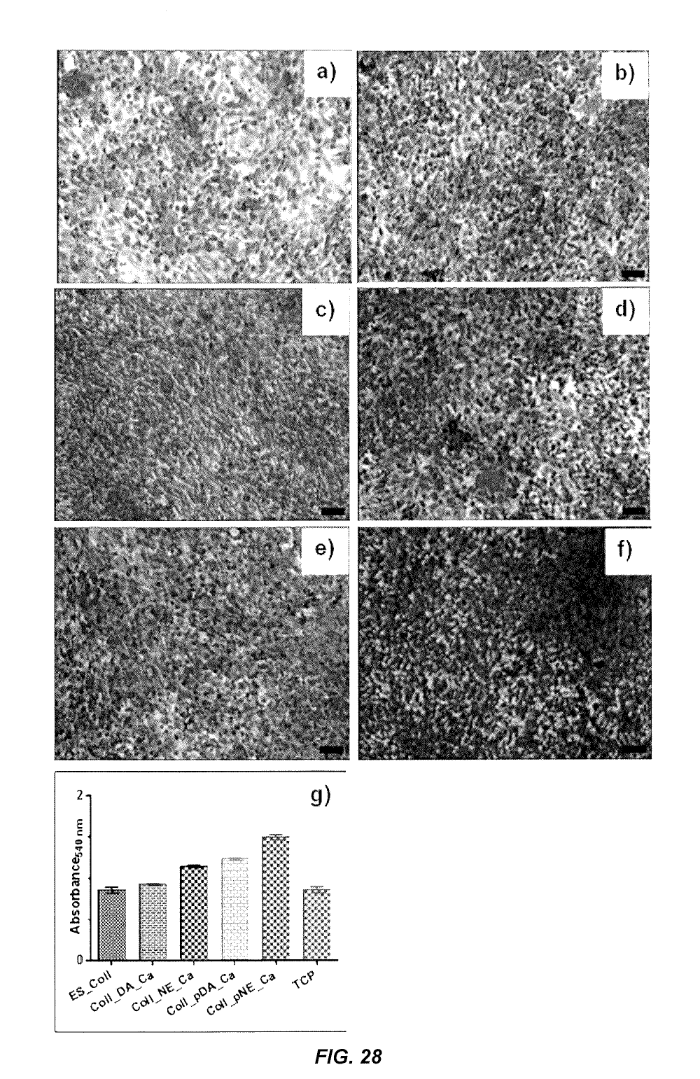

Upon osteoblast differentiation, the hFob cells enter into the mineralization phase to deposit the mineralized ECM. The capacity of hFob to deposit minerals is a marker for osteogenic efficiency and can be monitored by ARS staining of the cells cultured on different scaffolds after 9 days p.s. FIG. 28 shows the optical images of various samples stained with ARS, wherein the bright red staining indicates the calcium mineralization due to ARS binding. When compared to TCP, all the electrospun mats displayed substantial ARS staining, indicating increased mineralization of the scaffolds. ARS staining was enhanced in the mats containing catecholamine/Ca²⁺ and more pronounced in the polycatecholamines-CaCO₃ composite mats than in ES_Coll, consistent with the increased ALP activity observed on these scaffolds. ARS stained optical images further showed thick bone nodule formation on hFob cells cultured on Coll_DA/NE_Ca or Coll_pDA/pNE_Ca mats FIGS. 25C-25F). These results suggest that the biochemical cues arising from polycatecholamines-CaCO₃ composite structures stimulate the hFob adhesion, proliferation and differentiation. Quantitative estimation of the colorant on various mats by cetylpyridiniumchloride assay at 14 days p.s. further confirmed the increased osteogenic potential of Coll_pDA/pNE_Ca (FIG. 25G). Similar to the trend observed with the ALP activity, higher calcium nodules (p<0.0001) were observed for Coll_pNE_Ca than Coll_pDA_Ca (Table 9B).

TABLE 9B
Statistical comparison of the ARS data for various collagen
scaffolds and TCP at 14 Days post-seeding. Significance values:
*, p ≤ 0.05; **, p < 0.01; ***, p < 0.001; ****,
p < 0.0001 and ns, p > 0.05 by t-test or 1-way ANOVA.

| Scaffold Type | TCP | ES_Coll | Coll_DA_Ca |
|---|---|---|---|
| TCP | — | ns | ns |
| ES_Coll | ns | — | ns |
| Coll_DA_Ca | ns | ns | — |
| Coll_pDA_Ca | **** | **** | **** |
| Coll_NE_Ca | **** | **** | **** |
| Coll_pNE_Ca | **** | **** | **** |
| Scaffold Type | Coll_pDA_Ca | Coll_NE_Ca | Coll_pNE_Ca |
| TCP | **** | **** | **** |
| ES_Coll | **** | **** | **** |
| Coll_DA_Ca | **** | **** | **** |
| Coll_pDA_Ca | — | * | **** |
| Coll_NE_Ca | * | — | **** |
| Coll_pNE_Ca | **** | **** | — |

Morphology of Osteoblasts on the Collagen Fibres Scaffolds:

A morphological study of in vitro-cultured osteoblast was performed after 3 and 9 days of cell culture using SEM. After removal of non-adherent cells, the scaffolds with cells were fixed in 3% glutaraldehyde. The cell-scaffolds samples were then dehydrated using increasing concentrations of ethanol (30%, 50%, 75%, 90% and 100%) for 15 min each concentration and finally treated with hexamethyldisilazane and air-dried overnight in order to maintain normal cell morphology. Dried cellular constructs were sputter-coated with gold and observed under an SEM at an accelerating voltage of 10 kV.

Cell Morphology Analysis.

Cellular morphology of in vitro cultured hFob was analysed at 11 and 14 days p.s., by FE-SEM (FEI-QUANTA 200F). Cell-seeded scaffolds were washed with PBS to remove the non-adherent cells and fixed by using 3% glutaraldehyde at room temperature. The cell scaffolds were then dehydrated using a series of graded alcohol solutions and finally dried into HMDS overnight. Dried cellular constructs were then sputter-coated with platinum and observed under FE-SEM at an accelerating voltage of 10 KV.

Scanning Electron Microscopy:

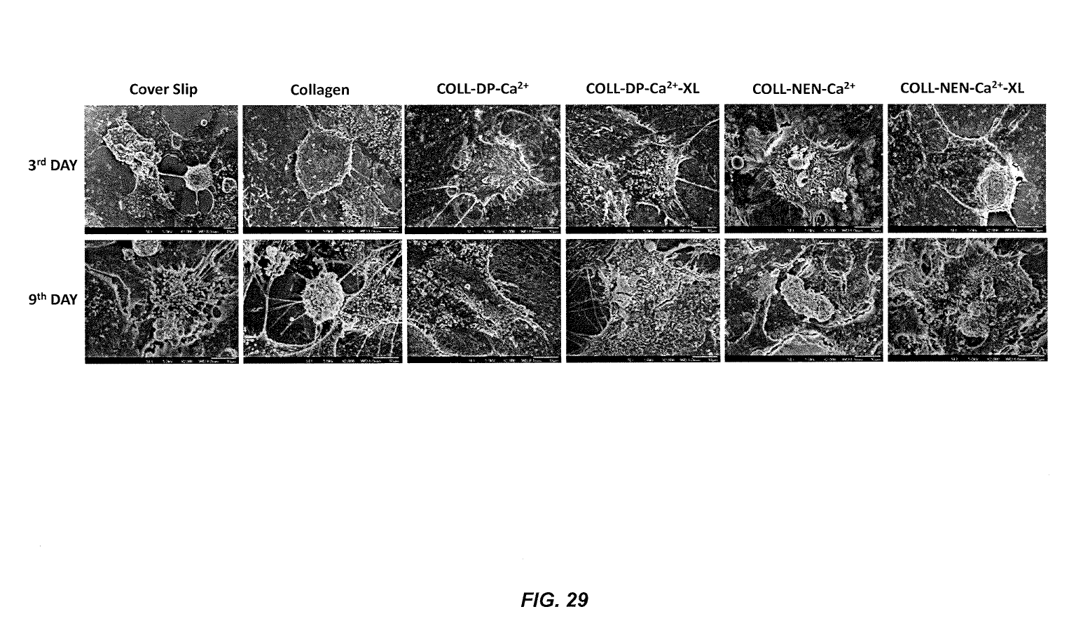

SEM images of the hFOBs cultured on various ES scaffolds are shown in FIG. 29. In augment with the ARS staining and ALP activity tests, an extensive cell adhesion, spreading was observed after 3 days and extensive mineralization was observed after 9 days in ES collagen containing catecholamine-Ca²⁺ after exposure to gaseous ammonia.

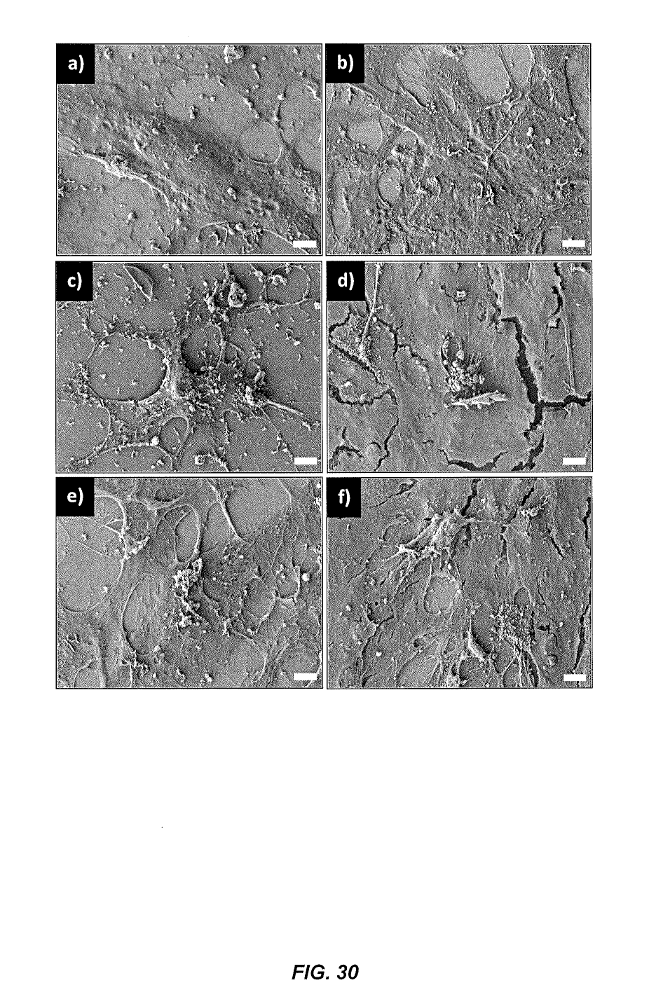

Cellular morphology of the hFob seeded on various scaffolds were visualized (9 days p.s.) by FE-SEM (FIGS. 30A-30F). The results indicated enhanced cell attachment and cell spreading on various collagen scaffolds with the formation of mineral particles. The morphology of cells seeded on Coll_pDA_Ca and Coll_pNE_Ca scaffolds indicated the presence of more consolidated layers of fully extended cells interconnected by lamellipodia and secretion of ECM (FIGS. 30E, 30F). An abundant mass of fibers beneath the cells indicated the deposition of ECM, confirming cell proliferation and mineralization. The overall obtained results suggested that the mineralized scaffolds promote better biochemical cues for cellular adhesion, proliferation, mineralization and enhanced expression of osteo-specific proteins proved the potential applications in bone tissue regeneration.

Immunofluorescent Staining for Osteocalcin (OC), Osteopontin (OPN) and Bone Morphogenetic Protein 2 (BMP-2) and Western Blot Analysis.

The hFob cells cultured on various scaffolds and CS were processed for protein staining 11 days p.s. Cells were fixed in 4% (v/v) formaldehyde for 10 min, followed by permeabilization using 0.3% Triton X-100 for 5 min. Cells were then labeled with bone specific markers: anti-osteocalcin (OCN), anti-osteopontin (OPN) and anti-bone morphogenetic protein 2 (BMP-2) antibodies. Hoechst was used to visualize the nucleus. Confocal images were acquired using Zeiss LSM800 Airyscan 40× oil immersion objective lens as described earlier.

Western blot analysis was done for the quantitative estimation of the osteopontin protein generated over different scaffold types at day 11 p.s. The hFob cells were lysed in ice-cold lysis buffer containing Triton X-100 (1%) and protease inhibitors. Detergent-insoluble material and nuclei were removed by centrifugation for 5 min at 10000 rpm. The protein content of the cell lysates was determined by the Bio-Rad Protein Assay. Equal amount of cell lysates were resolved on SDS-PAGE gels and subsequently transferred onto polyvinylidene difluoride (PVDF) membranes. After blocking in 5% milk powder in TBS-0.05% Tween 20 (TBST) for 1 h at room temperature, the membranes were washed three times in TBST. The membranes were then incubated overnight at 4° C. with diluted primary antibody in 5% milk with gentle rocking. After three washes in TBST, the membranes were incubated with HRP-conjugated secondary antibody for 1 h at room temperature. After three further washes in TBST, the membranes were incubated with the LumiGLO® chemiluminescent detection system (Cell Signalling Technology) and exposed to light sensitive film for various times. Densitometric analyses of the Western blots were performed using ImageJ software.

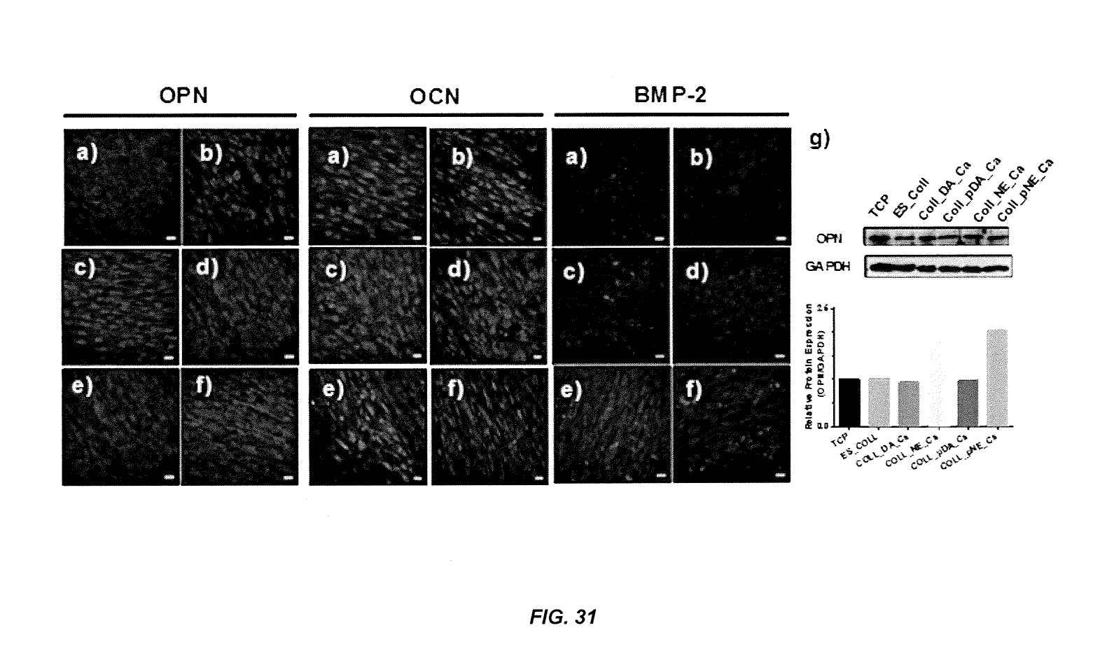

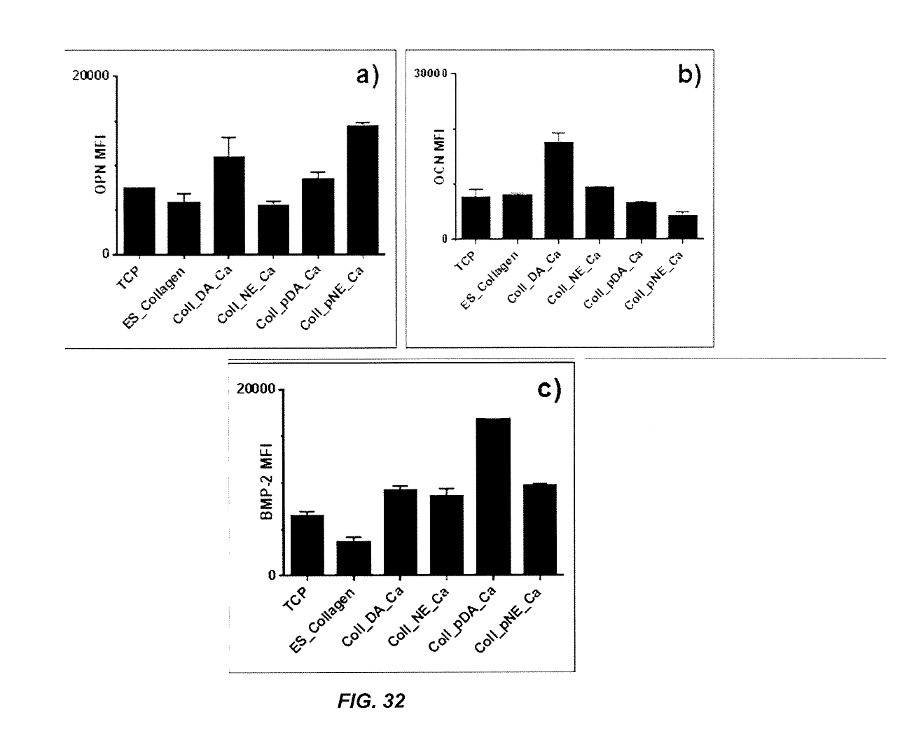

To obtain further insight into the biochemical cues on scaffolds-cell interactions, we determined the expression of key osteo-specific marker proteins such as osteocalcin (OCN), osteopontin (OPN) and bone morphogenetic protein (BMP) by immunofluorescent labelling (FIG. 31). As observed in MTS/ALP assays and in ARS staining, an increased immunofluorescence was observed for all the marker proteins in the electrospun fibers compared to cells cultured on TCP. Immunofluorescence studies further confirmed the elevated levels of OPN, OCN and BMP on the catecholamines cross-linked nanofibrous mats than the TCP and ES_Coll scaffolds (FIG. 31). The osteogenic marker proteins osteopontin, osteocalcin and bone morphogenetic protein expression was increased in Coll_pDA_Ca and Coll_pNE_Ca than in ES_Coll or Coll_DA/NE_Ca scaffolds (FIGS. 31A-31F). A semi-quantitative analysis has been carried out based on the staining intensity of osteogenic markers. When compared to TCP, ES_Coll and other electrospun mats, the expression of OPN was more pronounced on cells seeded on Coll_pNE_Ca mats (FIG. 32A). Similarly, a higher BMP-2 expression levels was observed on cells seeded on Coll_pNE_Ca mats (FIG. 32B). Finally, we have used Western blotting to confirm the differences in the expression of OPN by cells seeded on various mats. As shown in (FIG. 31G cells seeded on Coll_NE_Ca or Coll_pNE_Ca mats displayed 1.8-2 fold higher expression of OPN than cells seeded on other scaffolds. Together with immunostaining, these results demonstrate excellent osteoconductive properties of norepinephrine-Ca²⁺ composite structures.

Effect of Ca²⁺ on Dopamine Polymerization:

The conversion of dopamine to polydopamine was monitored spectrophotometrically by monitoring the changes in the absorption of UV spectra of dopamine. Briefly, dopamine was dissolved in Tris-HCl buffer (pH 8.5) and the changes in spectra was recorded at a predetermined intervals. To study the effect of Ca²⁺ on dopamine polymerization, calcium chloride (25 mM) was added to the Tris-HCl buffer containing the same amount dopamine, and the spectra was recorded using the same pre-determined intervals as before.

Addition of Ca²⁺ Accelerates the Polydopamine Formation:

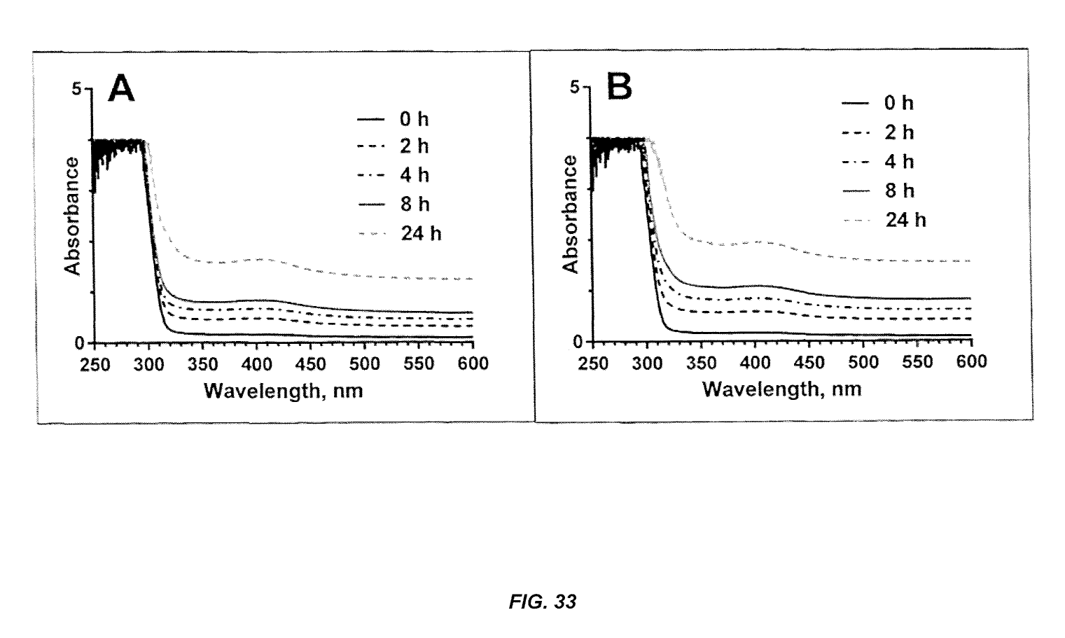

To confirm the effect of Ca²⁺ ions on dopamine polymerization, we recorded the progressive changes in the UV absorption spectra of dopamine in Tris HCl buffer (pH 8.5) as well as the dopamine in Tris HCl buffer containing 25 mM of CaCl₂. As shown in FIGS. 33A and 33B, an increased absorption at around 400 nm was observed in the buffer containing Ca²⁺ ions at all-time intervals, indicating accelerated formation of polydopamine intermediates.

### Example 11. Cytotoxicity Analysis of Polymer Products and Preparation of Durable Antimicrobial Wound Dressings

Primary human dermal fibroblasts (hDFs) cells were cultured in DMEM medium supplemented with 10% (v/v) fetal bovine serum, 50 U mL-1 penicillin and 50 μg mL⁻¹ streptomycin in a humidified incubator at 37° C. and 5% CO2. The cells (1×10⁵ cells well⁻¹) were seeded onto the ES fiber mats prepared on coverslips, placed at the bottom of the 12-well plates (Nunc®) and allowed to adhere and grow for 24 h before analysis. After washing with phosphate buffered saline (PBS), the cultured cells were fixed in 3% paraformaldehyde, and then fluorescently labeled with FITC conjugated anti-α-tubulin and Alexa Fluor 569 phalloidin (Molecular Probes®) to visualize cellular morphologies and Hoechst to visualize the nuclei. Coverslips were mounted on glass slides using Flouromount™. Confocal imaging was carried out by a laser scanning microscope (Zeiss LSM710-Meta, Carl Zeiss Microimaging Inc., NY, USA) using a 40× oil immersion objective lens and imaged as before. At least 20 different microscopic fields were analyzed for each samples.

Cell viability was determined using CellTier 96® Aqueous One solution cell proliferation assay kit according to the manufacturer's instruction. Briefly, at the end of the treatment period, cells growing on the scaffold-coated coverslips placed in a 12-well plate containing 500 μL of cell culture medium were incubated with 50 μL of MTS tetrazolium solution (provided by manufacturer) for 2 h at 37° C. Metabolically active cells reacted with the tetrazolium salt present in MTS reagent producing a soluble purple formazan dye with absorbtion maxima at 490 nm. Subsequently, the absorbance was measured at 490 nm using a microplate reader (Infinite M200 Pro, Tecan, Mannedorf, Switzerland) and then relative cell viability was calculated. Each treatment was performed in three independent triplicates.

### Results

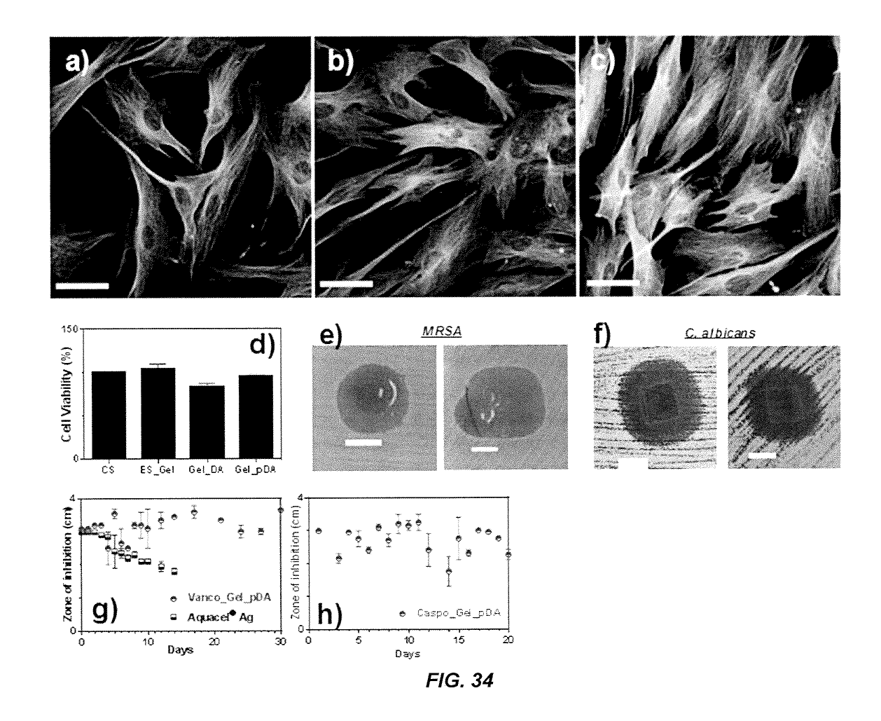

Previous studies have demonstrated that pDA coating can be utilized to immobilize the surface of electrospun nanofibers with cell adhesion peptides, growth factors, bone matrix protein-2 and bone forming peptide-1 through immine functionalization or Michael addition reactions₄₃₋₄₆. Having verified the generation of pDA crosslinking and the formation of osteoconductive scaffolds by ADM, we next evaluated the prospective utility of our approach in the preparation of durable wound dressings for biomedical applications. We used ES_Gel mats for this purpose owing to their numerous advantages such as good biocompatibility, biodegradability, absorbency, and cell adhesion properties, easy availability, and low cost. We first examined the cytotoxicity of the mats against primary human dermal fibroblasts (hDFs), a well-characterized and widely used model system for determining skin biocompatibility of dressings and implants. Exposure of hDFs to ES_Gel or Gel_pDA mats for 24 h failed to alter cellular or nuclear morphology, cytoskeletal architecture, or cell adhesion properties (FIGS. 34A-34C). An MTS (3-(4,5-dimethylthiazol-2-yl)-5-(3-carboxymethoxyphenyl)-2-(4-sulfophenyl)-2Htetrazolium)-based chromogenic assay further showed that pDA coating of the gelatin mats did not interfere with hDF viability, confirming the biocompatibility of the crosslinked scaffold (FIG. 34D). Therefore, the oxidative polymerization of DA-loaded electrospun gelatin nanofibers by ADM yields a benign, non-cytotoxic pDA coating on the fibers.

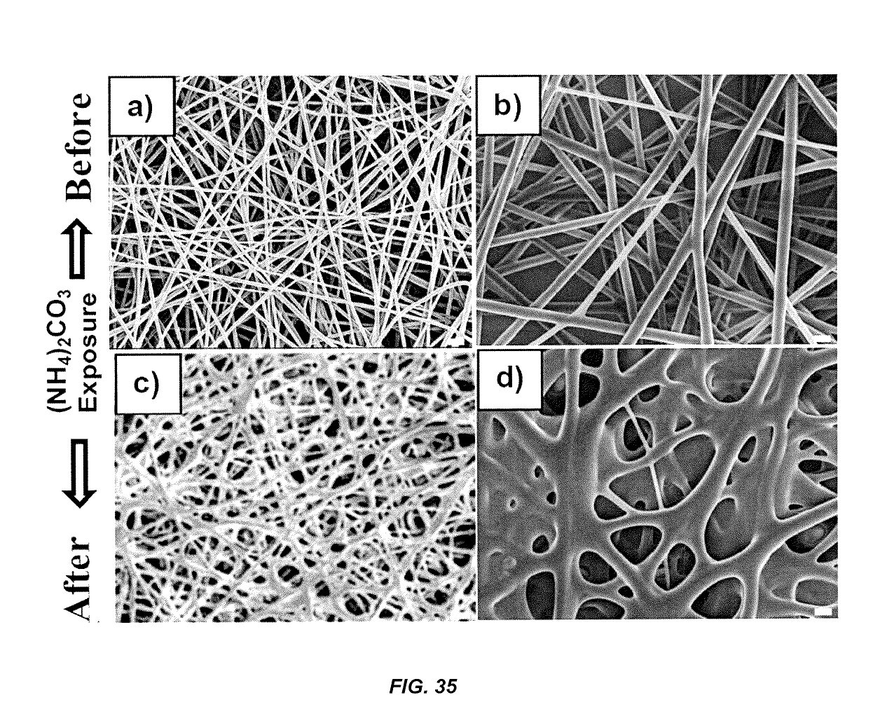

We selected two US FDA approved lipopeptide antibiotics, vancomycin and caspofungin, to assess the antimicrobial efficacy of the pDA-coated electrospun nanofiber mats. Vancomycin is a mainstay therapeutic agent for the treatment of invasive infections against the “superbug”, methicillin-resistant S. aureus (MRSA)₄₇, whereas caspofungin is approved for the treatment of invasive fungal infections and for patients who are allergic to amphotericin B and itraconazole antifungal medications₄₈. For the preparation of antibiotics loaded mats, vancomycin or caspofungin (0.5% w/w of Gel) was added to Gel-DA dope solution. Since the addition of vancomycin caused substantial solution turbidity, electrospinning was carried out in 80% TFE at 14 KV. Incorporation of antibiotics at a concentration of 0.5% (w/w) onto gelatin nanofibers did not alter fiber morphology before or after ADM (FIGS. 35A-35D). The concentration of the antibiotics was chosen to maintain the antimicrobial component at 50% lower than that maintained by the commercial Ag-based wound dressings, Aquacel® Ag and Melgisorb®. To confirm the retention of antibiotic activity post-incorporation, a disc diffusion assay was performed in accordance with the guidelines of the Clinical and Laboratory Standard Institute. Both vancomycin- and caspofungin-loaded Gel_pDA (Vanco_Gel_pDA and Caspo_Gel_pDA, respectively) mats displayed excellent zones of inhibition against pathogenic Gram-positive S. aureus, MRSA, and assorted C. albicans bacterial strains compared with Aquacel® Ag and Melgisorb® dressings (FIGS. 34E, 34F), indicating that the crosslinking process did not impair antimicrobial properties.

To determine whether adherent pDA crosslinking can maintain long-term antimicrobial activity, we tested the durability of the mats to leaching in accordance with the guidelines of the American Society for Testing and Materials for antimicrobial-coated medical devices₄₉. Briefly, Vanco_Gel_pDA and Caspo_Gel_pDA mats were immersed in phosphate-buffered saline (PBS; pH 7.0) with constant shaking. After the indicated intervals, the mats were removed, washed with water, and assayed by the disc diffusion method. A complete retention of antimicrobial activity was observed in both types of antibiotic-loaded mats even after 20 days of immersion in PBS (FIGS. 34G, 34H. In particular, the vancomycin-loaded mas exhibited superior retention of anti-MRSA activity relative to that exhibited by the commercial Ag-based wound dressing, Aquacel® Ag (FIG. 34G). Previous studies reported the occurrence of a ˜80-90% weight loss in glutaraldehyde-crosslinked electrospun gelatin mats within <5 h of incubation with PBS, indicative of leaching₅₀. These results confirm the excellent aqueous stability and durability of the antibiotic-loaded mats to leaching conferred by pDA crosslinking. In addition, performance of the entire crosslinking process in the solid phase permitted a high encapsulation efficiency of the antibiotics as well.

### Example 12. In Vivo Porcine Skin Model of Burn Wound Healing

Burns were created under the anaesthetic conditions on the pig skin via direct contact with a hot water beaker preheated to 92° C. for 15 s. Eight second degree burns, up to dermis layer, were created on the thoracic ribs of each pig. Burns were created 4 on each side, i.e. four on the cranial end and the four on the caudal end, with 1 cm distance in-between. During burn creation, surgical drapes with absorbent pads were used around animal to avoid spillage and leakage from the burning procedure. In context to the circular burning device, we have used 100 ml beaker (Diameter ˜4 cm, Surface area ˜12.57 cm₂) filled with 50 ml sterile water maintained at 92° C. The pressure to the burning device was induced using 500 ml schott duran bottle filled with 300 ml warm water (Temp ˜45° C.). The burning device was maintained in contact with the porcine skin for an optimized time of 15 s to generate uniform burn wounds. The created wounds were photographed immediately to estimate the initial wound area for each burn.

The wounds were cleaned to remove the burned epithelium debris. The test wound dressings including ES_Gel, vanco_Gel_pDA and Aquacel® Ag were applied on the designated wounds, two wounds for each dressing type, to completely cover the wound area. Two wounds were left uncovered and were labelled as untreated control burn wounds. To prevent inter-wound cross contamination, Tegaderm films were used for covering the wound dressing area. Dressings were changed at the frequency of twice a week under anaesthetic conditions. Animal was first sedated with an intramuscular dose of 40% ketamine/xylazine to induce anaesthesia (13 mg kg₋₁ ketamine/1 mg kg₋₁ xylasine) and maintained with 1-2% isofluorane. To reduce the discomfort, buprenorphrine (0.01 mg kg₋₁) was applied intramuscularly before and 2 days after the burn wounds. At the time of dressing change, wounds were washed with 0.05% chlorhexidine solution and cotton gauze before placing fresh dressing material. The wounds were examined and a clinical description of the wound was noted. Photographs were taken from the wounds of all the groups, using a Nikon D90 digital SLR camera. To ensure the camera was at a standard distance from the wound, a template was used to mark four dots on the skin surface, and were lined up with the focusing spots inside the camera viewfinder. A Cyan-Magenta-Yellow-Black (CMYK) colour scale was placed beside the wound so that the colours in the photos could be standardised against each other. The SigmaScan Pro 5 software was used to calculate the total wound area in square centimetre for different wound dressings.

Wound Healing Efficacy of Vancomycin-Loaded, pDA-Crosslinked Mats in a Porcine Deep Dermal Burn Injury Model. Finally, we evaluated the wound healing properties of pristine ES_Gel and Vanco_Gel_pDA mats using a porcine deep dermal burn wound healing model. In terms of anatomical and physiological properties, porcine skin closely resembles human skin, and the wound healing properties of the pig are considered similar to those of the humans₅₁. Here, burns were created under anesthetic conditions by placing a hot water beaker preheated to 92° C. for 15 sec on the dorsum of the animals. Eight second degree burns (up to the dermal layer) were created on the thoracic ribs of each pig. Aquacel® Ag-treated and untreated wounds served as positive and negative controls, respectively. Photographs were taken of the wounds with a Nikon D90 digital SLR camera, and the images were processed using SigmaScan Pro 5 software. For each wound at every time point, the wound size was measured and compared with that of untreated control wounds.

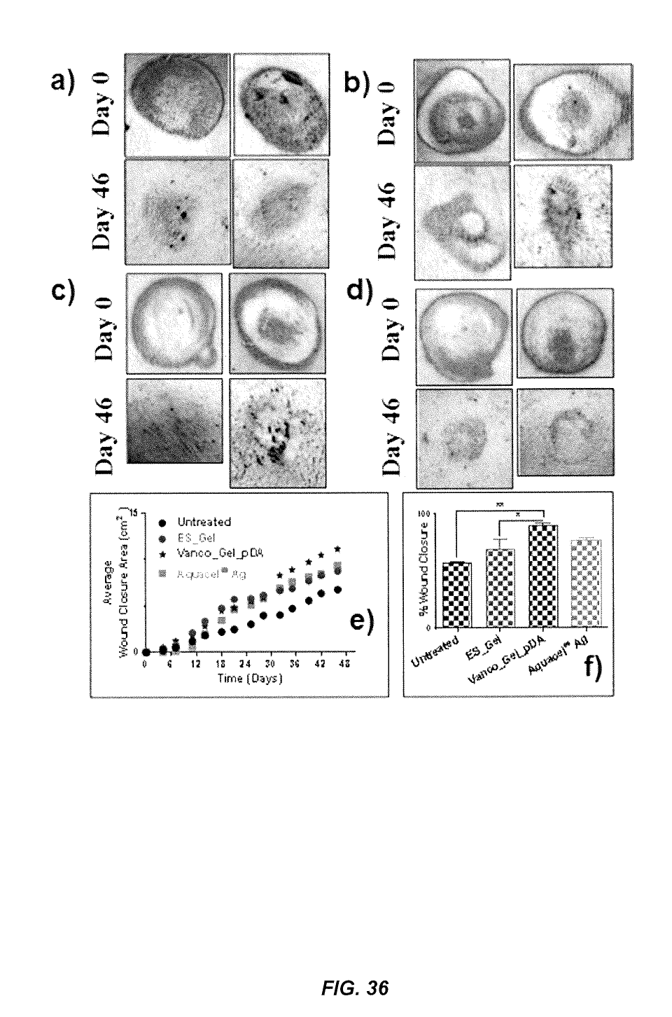

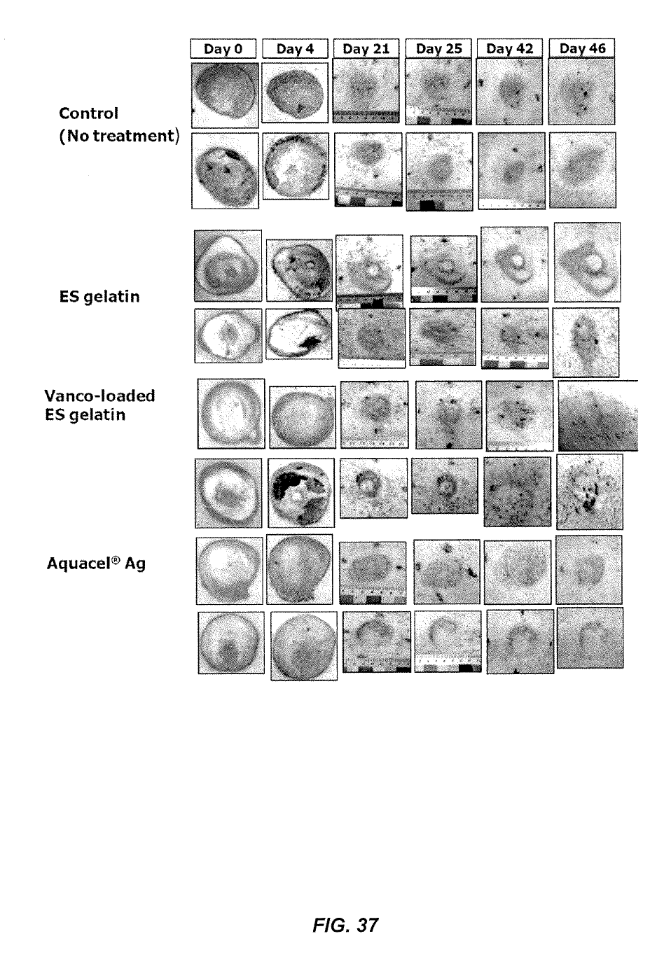

FIGS. 36A-36D show the wound area at the beginning (day 0) and the end (day 46) of the wound healing process for all four wound groups (ES_Gel, Vanco_Gel_pDA, Aquacel® Ag, and untreated control). Photographs displaying the progression of wound healing events among the various groups are shown in FIG. 37. The total wound size reduction was plotted as a percentage of the initial wound size (FIGS. 36E, 36F). Consequently, an increased wound closure (89.6%) was observed for burn wounds treated with Vanco_Gel_pDA mats versus untreated control wounds (p≤0.01) or wounds treated with pristine ES_Gel mats (p≤0.05). Additionally, a faster wound healing rate was observed for wounds treated with Vanco_Gel_pDA mats than for the other three groups at 2 weeks after injury. Moreover, the final wound closure area displayed improvements compared with those afforded by the commercial Ag-based dressing. These results indicate that antibiotic-loaded, pDA-crosslinked electrospun nanofibrous mats promote rapid re-epithelialization of burn wounds, and establish the in vivo efficacy and biocompatibility of the scaffold.

### V. References

Abrigo et al., (2014); Macromol. Biosci. 14:772-92
ASTM, Standard Test Method for Determining the Antimicrobial Activity of Antimicrobial Agents Under Dynamic Contact Conditions, E2149-13a.
Baltrusaitis et al., Phys. Chem. Chem. Phys. 2007, 9, 3011-3024.
Bhattacharya et al; J Biomed Nanotechnol. 2009 February; 5(1):69-75.
Blanchard et al., Surf. Sci. 1992, 276, 27-39.
Chai et al., ACS Appl. Mater. Interfaces 2014, 6, 17974-17984.
Chu et al., RSC Adv. 2013, 3, 21722-21729.
Connolly, Clin Orthop Relat Res. 1995 April; (313):8-18.
Demri et al., J. Mater. Process. Technol. 1995, 55, 311-314.
Deng et al., Adv. Funct. Mater., vol. 21, pp. 2641-2651, 2011.
Deng et al., IEEE Trans Nanobioscience. 2012 March; 11(1):3-14.
Dreyer et al., (2012) Langmuir 28:6428-6435
Du et al., Adv Mater. 2014; 26:8029-33.
Flatley et al., Clin Orthop Relat Res. 1983 October; (179):246-52.
Frohbergh et al., Biomaterials 2012, 33, 9167-9178.
Fujihara et al., Biomaterials 2005, 26, 4139-4147.
Gao et al., J. Appl. Polym. Sci. 2013, 127, 2588-2599.
Gupta et al., Biomaterials 2009, 30, 2085-2094.
He et al., Int. J. Biol. Macromol. 2011, 48, 354-359.
He et al., J. R. Soc. Interface 2014, 11, 20140169.
Herron et al., Spine (Phila Pa. 1976). 1989 May; 14(5):496-500.
Hong et al., (2011) Nanomed 6:793-801
Hong et al., (2013) Angew Chem Int 52:9187-9191
Hu et al., Cosmochim. Acta 2006, 70, 3342-3350.
Joshi et al., (2014) Int. J. Nanomed. 9:2439-58
Kaban et al., J Oral Maxillofac Surg. 1982 October; 40(10):623-6.
Kale et al., Am J Orthop (Belle Mead N.J.). 1995 October; 24(10):752-61.
Kharaziha et al., J. Mech. Behav. Biomed. Mater. 2013, 24, 9-20.
Kim et al., Adv. Funct. Mater. 2005, 15, 1988-1994.
Kouhi et al., Chem. Eng. J. 2013, 228, 1057-1065.
Ku et al., (2010) Biomaterials 31(36): 9431-9437
Lakshiminarayanan et al., (2014) J. Nanosci. Nanotechnol. 14:853-67
Lee et al., Science. 2007; 318:426-30.
Li et al., J. Biomed. Mater. Res., vol. 60, pp. 613-621, 2002.
Li et al., Langmuir. 2016, 32, 3226-33.
Li et al., Sens. Actuat. B-Chem. 2010, 151, 30-38.
Liu et al., Biomaterials. 2007 January; 28(2):354-69.
Luo et al., Colloids Surf B Biointerfaces. 2013 Jun. 1; 106:66-73.
Matthews et al., Biomacromolecules 2002, 3, 232-238.
Mulliken et al., Ann Surg. 1981 September; 194(3):366-72.
Nair et al., Expert Opin. Biol. Ther., vol. 4, pp. 659-668, 2004.
Pelled et al., J Biomech. 2007; 40(2):399-411
Prabhakaran et al., Acta Biomater. 2009, 5, 2884-2893.
Rim et al., (2012) Colloids Surfaces B 91:189-197
Robinson et al., Clin. Chem. 2003, 49, 1763-1773.
Shima et al., J Neurosurg. 1979 October; 51(4):533-8.
Shin et al., (2011) Macromol Res 19(8): 835-842
Son et al., (2013) Macromol Mater Eng. 298: 547-554
Tian et al., Polymer (Guildf) 2007, 48, 2720-2728.
US 2014/0142025
Vacchia et al., (2013) Adv Funct Mater 23:1331-1340
Vidal et al., Acta Biomater. 2014, 10, 3288-3294.
Wang et al., Biomaterials. 2014 September; 35(27):7679-89.
Wei et al., (2010) Polym Chem 2010; 1: 1430-1433
Woo et al., J. Biomed. Mater. Res. A, vol. 67A, pp. 531-537, 2003.
Wutticharoenmongkol et al., Macromol. Biosci. 2006, 6, 70-77.
Xie et al., Adv Mater. 2014, 26, 4491-6.
Xie et al., (2012) J Biomed Mater Res Part A: 100A: 929-938
Yang et al., Proc Natl Acad Sci USA. 2009, 106, 10086-91.
Zangmeister et al., Langmuir 2013, 29, 8619-8628.
Zeugolis et al., Biomaterials 2008, 29, 2293-2305.
Zhao et al., Macromol. Rapid Commun. 2015, 36, 909-915.

Although the foregoing has been described in some detail by way of illustration and example for purposes of clarity and understanding, one of skill in the art will appreciate that certain changes and modifications can be practiced within the scope of the appended claims. In addition, each reference provided herein is incorporated by reference in its entirety to the same extent as if each reference was individually incorporated by reference. Any listing or discussion of an apparently prior-published document in this specification should not necessarily be taken as an acknowledgement that the document is part of the state of the art or is common general knowledge.

1. A method for preparing a polymer product comprising
i) electrospinning from a dope solution comprising at least one polymer and at least one cross-linking agent to prepare the polymer product,
wherein the cross-linking agent comprises at least one catecholamine, and
ii) exposing the polymer product to at least one gaseous alkaline reagent.

2. The method according to claim 1, wherein the catecholamine comprises at least one catecholamine selected from the group consisting of dopamine, norepinephrine, adrenalone, carbidopa, colterol, L- or D-dihydroxy phenylalanine (dopa), dimethyldopa, dioxifedrine, dioxethedrin, 5-hydroxydopamine hydrochloride, dobutamine, dopexamine, droxydopam, α-methylnorepinephrine, ethylnorepinephrine, etilveodopa, isoetharine, hexaprenaline, N-methyladrenalone, norbudrine, nordefrin, oxidopamine and enterobactin.

3. (canceled)

4. The method according to claim 1, wherein the electrospun fibers can be collected on a bare metallic collector or on a fabric.

5. (canceled)

6. The method according to claim 4, wherein the fabric is a non-woven fabric or a bandage gauze.

7. (canceled)

8. The method according to claim 1, wherein the cross-linking agent further comprises at least one polyphenol.

9. (canceled)

10. The method according to claim 8, wherein the polyphenol comprises at least one polyphenol selected from the group consisting of hydroquinone, phloroglucinol, pyrogallol, gallic acid, nordihydroguaiaretic acid, γ-mangostin and α-mangostin.

11. (canceled)

12. The method according to claim 1, wherein the dope solution further comprises at least one polyhydroxy antimicrobial agent, at least one polyamine antimicrobial agent, or a combination thereof.

13-15. (canceled)

16. The method according to claim 12, wherein the polyhydroxy antimicrobial agent comprises amphotericin B, natamycin, nystatin, caspofungin, or a combination thereof.

17. The method according to claim 12, wherein the antimicrobial agent comprises vancomycin, polymycin B, daptomycin, ramoplanin A2, Ristomycin monosulfate, Bleomycin sulfate, phleomycin, amikacin, streptomycin, gentamycin, kanamycin, tobramycin, azithromycin, dirtythromycin, rifampicin, rifamycin, rifapentine, rifaximin, clarithromycin, clindamycin, kendomycin, bafilomycin, chlortetracycline, doxorubicin, doxycycline, tetracycline, 1-deoxynojirimycin, 1-deoxymannojirimycin, N-methyl-1-deoxynojirimycin, daptomycin, collistin, polymyxin B, or a combination thereof.

18. The method according to claim 12, wherein the polyamine antimicrobial agent comprises ε-polylysine, poly-L-lysine, poly-D-lysine and poly-L-ornithine, linear polyethylenimines, branched polyethylenimines, or a combination thereof.

19. The method according to claim 1, wherein the dope solution further comprises at least one metal ion.

20. (canceled)

21. The method according to claim 19, wherein the metal ion comprises a transition metal ion.

22-24. (canceled)

25. The method according to claim 19, wherein the metal ion comprises at least one metal ion selected from the group consisting of Ca, Zn, Fe, Co, Mg, Ni, Ag, Au, Cu, and Mn ions.

26-27. (canceled)

28. The method according to claim 1, wherein the polymer in the dope solution comprises a hydrophilic polymer, a water-soluble polymer, a biodegradable polymer, or a combination thereof.

29. (canceled)

30. The method according to claim 28, wherein the polymer comprises gelatin, collagen, bovine serum albumin, casein, zein, laminin, polyvinyl alcohol (PVA), polyacrylic acid, chitosan, poly lactide (PLA), poly(ε-caprolactone) (PCL), polyethylene oxide (PEO), poly-(lactide-co-glycolide) (PLGA), or a combination thereof.

31-32. (canceled)

33. The method according to claim 1, wherein the dope solution comprises about 2-30% w/v polymer.

34. The method according to claim 1, wherein the amount of cross-linking agent is less than the amount of polymer in the dope solution.

35. (canceled)

36. The method according to claim 1, wherein step ii) comprises exposing the polymer product to at least one gaseous alkaline reagent in the presence of a buffering agent.

37-49. (canceled)

50. A polymer product comprising polymeric fibers with cross-links of a polyphenolic compound, a catecholamine compound, a polymeric catecholamine compound, or a combination thereof.

51. (canceled)

52. The polymer product according to claim 50, comprising a fiber mat.

53. The polymer product according to claim 50, further comprising cross-links of at least one polyhydroxy antimicrobial compound, at least one polyamine antimicrobial compound, or a combination thereof.

54-59. (canceled)

60. The polymer product according to claim 50, wherein the polymer comprises gelatin, collagen, bovine serum albumin, casein, zein, laminin, polyvinyl alcohol (PVA), polyacrylic acid, chitosan, poly lactide (PLA), poly(ε-caprolactone) (PCL), polyethylene oxide (PEO), poly-(lactide-co-glycolide) (PLGA), or a combination thereof.

61-67. (canceled)

68. A polymeric dope solution comprising at least one polymer and at least one cross-linking agent, wherein the cross-linking agent comprises at least one polyphenol, at least one catecholamine, or a combination thereof.

69. (canceled)

70. The polymeric dope solution according to claim 68, wherein the catecholamine comprises at least one catecholamine selected from the group consisting of dopamine, norepinephrine, adrenalone, carbidopa, colterol, L- or D-dihydroxy phenylalanine (dopa), dimethyldopa, dioxifedrine, dioxethedrin, 5-hydroxydopamine hydrochloride, dobutamine, dopexamine, droxydopam, α-methylnorepinephrine, ethylnorepinephrine, etilveodopa, isoetharine, hexaprenaline, N-methyladrenalone, norbudrine, nordefrin, oxidopamine and enterobactin.

71. The polymeric dope solution according to claim 68, wherein the polyphenol comprises at least one polyphenol selected from the group consisting of hydroquinone, phloroglucinol, pyrogallol, gallic acid, nordihydroguaiaretic acid, γ-mangostin and α-mangostin.

72. (canceled)

73. The polymeric dope solution according to claim 68, wherein the polymer comprises gelatin, collagen, bovine serum albumin, casein, zein, laminin, polyvinyl alcohol (PVA), polyacrylic acid, chitosan, poly lactide (PLA), poly(ε-caprolactone) (PCL), polyethylene oxide (PEO), poly-(lactide-co-glycolide) (PLGA), or a combination thereof.

74-84. (canceled)

85. The polymeric dope solution according to claim 68, further comprising at least one metal ion.

86. (canceled)

87. The polymeric dope solution according to claim 85, wherein the metal ion comprises a transition metal ion.

88-90. (canceled)

91. The polymeric dope solution according to claim 85, wherein the metal ion comprises at least one ion selected from the group consisting of Ca, Zn, Fe, Co, Mg, Ni, Ag, Au, Cu and Mn ions.

92-95. (canceled)
